(19) United States
(12) Patent Application Publication
(10) Pub. No.: US 2026/0014096 A1
(43) Pub. Date: Jan. 15, 2026

(54) MEDICAL PATCH FOR CONTROLLED DELIVERY OF WOUND HEALING FACTORS AND THERAPIES

(71) Applicants: William Marsh Rice University, Houston, TX (US); Carnegie Mellon University, Pittsburgh, TX (US)

(72) Inventors: OMID VEISEH, Houston, TX (US); CHRISTIAN SCHREIB, Houston, TX (US); SUDIP MUKHERJEE, Houston, TX (US); ITZHAQ COHEN-KARNI, Pittsburgh, PA (US); MABEL BARTLETT, Pittsburgh, PA (US); YINGQIAO WANG, Pittsburgh, PA (US); LIYANG WANG, Pittsburgh, PA (US); SAMUEL GERSHANOK, Pittsburgh, PA (US)

(21) Appl. No.: 18/839,204

(22) PCT Filed: Feb. 17, 2023
(86) PCT No.: PCT/US2023/062829

§ 371 Date: Aug. 16, 2024

Related U.S. Application Data
(60) Provisional application No. 63/311,885, filed on Feb. 18, 2022.

(51) Int. Cl. A61K 9/70; A61B 5/145; A61B 5/1477; A61K 38/18; A61K 38/19; A61K 38/20; A61K 47/36; A61N 1/04; A61P 17/02

(52) U.S. Cl. CPC A61K 9/7084; A61B 5/14546; A61B 5/1477; A61K 38/185; A61K 38/191; A61K 38/2026; A61K 47/36; A61N 1/0468; A61N 1/0492; A61P 17/02

(57) ABSTRACT

The present disclosure is directed a medical device applied as patch to a wound to promote healing. Engineered cells are disposed in the device that can secrete wound healing factors in response to light utilizing optogenetics. Biomaterials can be formed as needles into which cells can be loaded. Biosensors can also be included to facilitate multi-point mapping of the wound healing process. Another feature is an electrical stimulator to enhance cell proliferation and differentiation, further enhancing wound repair.

### CLAIM OF PRIORITY

This application claims benefit of priority to U.S. Provisional Application Ser. No. 63/311,885, filed Feb. 18, 2022. The disclosure of the foregoing application is incorporated herein by reference in its entirety.

### STATEMENT REGARDING FEDERALLY FUNDED RESEARCH

This invention was made with government support under Grant No. D20AC00002 awarded by the Defense Advanced Research Projects Agency. The government has certain rights in the invention.

### BACKGROUND

### 1. Field of the Disclosure

The present disclosure relates generally to the fields of medicine, medical devices and wound therapy. More particular, the disclosure relates to wound patches to promote wound healing.

### 2. Background

Wound healing is a complex and essential process in the health and survival of nearly all higher organisms. The process consists of four tightly integrated and overlapping phases: hemostasis, inflammation, proliferation, and tissue remodeling or resolution. These phases and their associated functions should occur in the proper sequence, proper time, and proper duration to achieve the ideal outcome.

Unfortunately, there are many factors that can not only affect wound healing but that can impede one or more of the healing phases that, if unaddressed, and lead to improper or impaired tissue repair. In extreme cases, this can manifest itself systemic disease, permanent scarring, loss of function, and even death. A highly significant challenge lies in chronic/non-healing wound which causes pain, hospitalization and increased health costs. Improved approaches to wound healing, particularly in the chronic wound context, are therefore in great need.

### SUMMARY

The present disclosure features a wound healing device, such as a single or multi-component patch, comprising a first component comprising a chamber for housing a cell or cells. The device may further comprise living cells disposed in said first component, such as between about 10 and 10,000,000 cells/ml of said first component. The living cells may be engineered to secrete an agent, for example, a protein or small molecule. Exemplary agents include a growth factor, a cytokine, a lymphokine, a chemokine, or a neurotropic factor or hormone, such as interleukin 4 (IL-4), brain-derived neurotrophic factor (BDNF), tumor necrosis factor alpha (TNF-α), nerve growth factor (NGF), interleukin 12 (IL-12), interleukin 10 (IL-10), epidermal growth factor (EGF), fibroblast growth factor (FGF-2), platelet derived growth factor (PDGF), vascular endothelial growth factors (VEGF), interleukin 1 (IL-1), interleukin 6 (IL-6), connective tissue growth factor (CTGF), granulocyte-macrophage colony-stimulating factor (GM-CSF), leptin, adiponectin, interferon gamma-induced protein 10 (IP-10), nerve growth factor (NGF), insulin-like growth factor 1 (IGF-1), dopamine, acetylcholine, fractalkine, high mobility group box 1 (HMGB1), interleukin 1β (IL-1β), IL-1RA, interleukin 2 (IL-2), sIL-2Rα, interleukin 5 (IL-5), interleukin 7 (IL-7), interleukin 8 (IL-8, CCL8), interleukin 13 (IL-13), interleukin 15 (IL-15), interleukin 17A (IL-17A), interleukin 18 (IL-18), interferon-gamma (IFN-γ), monokine induced by gamma (MIG,CXCL9), macrophage inflammatory protein 1 alpha (MIP-1^{α}), chemokine (C-C motif) ligand 3CCL3), macrophage inflammatory protein 1 beta (MIP-1β,) chemokine (C-C motif) ligands 4CCL4), monocyte chemoattractant protein-1 (MCP-1), chemokine (C-C motif) ligand 2 (CCL2), macrophage colony-stimulating factor (M-CSF), Eotaxin (CCL11), active/latent transforming growth factor beta 1 (TGF-β1), and/or lactic acid other metabolites for glycolysis. The living cells may be mesenchymal stem cells, keratinocytes, fibroblasts, chondrocytes or retinal pigment epithelial cells.

The first component may comprise or be in the form of a chamber or array, such as a needle array, e.g., a microneedle array. The wound healing device may be formed from a first and a second component, said first component comprising, for example, a chamber for housing a cell, and a second component providing, for example, structural support to said first component. Alternatively, the wound healing device may be formed exclusively by said first component. The wound healing device may further comprise an immunomodulatory agent in either or both the first and/or second component to, for example, mitigate an immune response against the wound healing device when placed into contact with living tissue in a subject. The first component may comprise a biocompatible material, such as a hydrogel comprising a naturally occurring or non-naturally occurring substance. For example, the hydrogel may comprise an alginate, alginate-acrylamide, chitosan, alginate-gelatin, hyaluronic acid, chondroitin sulfate, polyethylene glycol (PEG), PEGylated fibronectin, or peptide gel. In an embodiment, the second component comprises a biocompatible material, such as a structurally supportive biocompatible material, e.g., polydimethylsiloxane (PDMS), polyimide, polyurethane, polyethylene or polytetrafluoroethylene (PTFE).

The cells, e.g., engineered cells, may secrete said growth factor, cytokine, lymphokine, chemokine, or peptide (a) constitutively, (b) in response to light, such as blue light or red light or (c) constitutively but secretion is increased in response to light, such as blue light, orange light, green light, violet light, near infrared or red light. The device may further comprise (a) a biomarker sensor to map wound healing and/or (b) an electrical stimulator, such as biomarker sensor that measures nitric oxide, a chemokine or a cytokine, e.g., TGF-β. The biomarker sensor may provide a spatial map of healing in said wound, said sensor optionally being operably connected to a light emitting device. The electrical stimulator may be a low impedance/high charge injection stimulator.

In another embodiment, there is provided a method of treating a wound in a subject (e.g., a living subject) comprising applying a wound healing device as described herein to a wound. Engineered cells in the device may secrete an agent, e.g., a growth factor, cytokine, lymphokine, chemokine, or neurotropic factor or hormone constitutively or in response to light, such as blue light, orange light, green light, violet light, near infrared or red light or constitutively but secretion can be increased in response to light, such as blue, orange light, green light, violet light, near infrared light or red light. The method may thus further comprise subjecting said wound healing device or portion thereof to light.

The wound healing device may further comprise a biomarker sensor to map wound healing, such as a nitric oxide, a chemokine or a cytokine, e.g., TGF-β, an electrical stimulator, or both. The biomarker sensor may provide a spatial map of healing in said wound, said sensor optionally being operably connected to a light emitting device. The electrical stimulator may be a low impedance/high charge injection stimulator. The method may further comprise applying an electrical current to said wound. The wound may be a skin wound, a muscle wound, a penetrating wound, a closed wound, an open wound, muscle loss, or organ damage, may be a chronic/non-healing wound, may be a traumatic wound or a surgical wound.

It is contemplated that any method or composition described herein can be implemented with respect to any other method or composition described herein. Other objects, features and advantages of the present disclosure will become apparent from the following Detailed Description, Drawings, Examples, and Claims. It should be understood, however, that the Detailed Description, Drawings, Examples, and Claims while indicating specific embodiments of the disclosure, are given by way of illustration only, since various changes and modifications within the spirit and scope of the disclosure will become apparent to those skilled in the art.

### BRIEF DESCRIPTION OF THE DRAWINGS

FIGS. 1A-1B demonstrate light-triggered protein production from optogenetic cells. FIG. 1A illustrates plasmid that were designed and are currently being fabricated. Shown is the plasmid for BDNF being driven by the EL222 blue-light responsive optogenetic system. Other embodiments comprise additional therapeutic proteins, e.g., interleukins, inter alia. FIG. 1B shows the PhyB/PIF6 red light responsive optogenetic system driving the reporter protein SEAP. Error bars represent standard deviation of the mean. This data suggests that there is increased protein production in response to red light; however, the system still constituitively produces protein without the red-light trigger. As such, this system may be exploited to drive a therapeutic protein that needs to be expressed at all times, but augmented expression is necessary at certain times; or, alternatively, to optimize conditions to reduce the background expression.

FIG. 2 is a graph of optogenetically activated TNF-α secretion. The results demonstrate that blue light triggered production of TNF-α. ELISAs were performed to prove that TNF-α can be controlled optogenetically in ARPE-19 cells, with a production rate of ˜0.012 pg TNF-α per hour after illumination with blue light. Cells were exposed to 1 hour of blue light and media and harvested 8 hours after exposure for assaying. Sham cells are identical to the optogenetically engineered cells, except that they lack the TNF-α gene driven by the optogenetic promoter.

FIGS. 3A-B illustrate the Gibson assembly method to fabricate red-light triggered plasmids. FIG. 3A depicts the Gibson Assembly protocol. DNA fragments corresponding to the genes for the therapeutic proteins (BDNF, IL4, NGF, and TNF) and the plasmid backbone from the red-light triggered system (NFAT-RE backbone) are amplified by PCR, and then assembled to construct the final plasmid. FIG. 3B shows an image of an agarose gel demonstrating that the DNA fragments of interest were PCR-amplified.

FIGS. 4A-B are images depicting initial 3D printing of an embodiment of the microneedle patch mold. These patch molds were 3D printed at Rice University employing the Formlabs© FORM2 printer.

FIGS. 5A-B illustrate sequencing data to validate fabricated optogenetic plasmids. FIG. 5A shows sample sequencing data. The sequence was validated by comparing the sequence determined experimentally from Fluorescent Dye Terminator Sequencing with the desired theoretical sequence. FIG. 5B gives a table of purity and sequencing data of the fabricated plasmids. “260/280” is the comparison of absorbance maxima at 260 nm and 280 nm; a ratio ˜1.9 is indicative of pure DNA. “% sequence matching” is the percentage of plasmids which match the desired sequence, with 100% being optimal. “Any inserts/deletions?” is an indication of any inserted or deleted sequences. Any insertion or deletion renders the plasmid nonfunctional; as such, validated plasmids must be free on nucleotide insertions and or deletions. *CAG-PhyB/PIF6 still needs to be sequence confirmed, however the cells engineered with this plasmid work as intended.

FIGS. 6A-B show graphs that demonstrate light triggered production of TNF-α, IL4, and BDNF. ELISAs were performed to prove that TNF-α, IL4, NGF, and BDNF may be controlled optogenetically in epithelial cells. Data is shown in pg/cell/hour of light exposure Cells were exposed to the indicated color of light for 5 minutes and media was harvested immediately for assaying. FIG. 6A shows red-light responsive cells, whereas FIG. 6B shows blue-light responsive cells.

FIG. 7 is a graph of BDNF secretion after illumination with blue light, demonstrating sustained release from light-triggered cells. Cells were seeded in a 48-well plate at 50,000 cells per well. After a media change, cells were left in the dark for 24 hours and media was replaced. New media was added, and the cells were then exposed to blue light for the indicated time on the x-axis. At the indicated time, media was harvested immediately from 4 wells and stored at 4° C. until the ELISA was performed. (N=4)

FIGS. 8A-B show cell viability and release data from cells encapsulated in alginate-acrylamide. FIG. 8A shows results from a representative cell viability/cytotoxicity assay employing LIVE/DEAD stain, which stains live cells green and dead cells red. These results suggest that the optogenetically engineered cells are both alive and viable inside the covalently crosslinked alginate-acrylamide. FIG. 8B show results from an ELISA assay for release of therapeutics from the hydrogel. Results compare cells on a TC plate (control), cells in ionically crosslinked alginate (control), and cells in covalently crosslinked alginate-acrylamide. Results suggest little change in secretion amounts between cells inside and outside of the crosslinked hydrogel. Signal in 0′ illumination group for alginate and alginate-acrylamide conditions are possibly from unintentional light exposure to the cells during the encapsulation process.

FIG. 9 show images of patch microneedle formation at various alginate volumetric weight percents employing two alginate viscosity grades. Shown in the drawing is a modulation of alginate concentrations to optimize microneedle formation in an embodiment of the inventive patch. Higher concentrations generally lead to stiffer hydrogels. SLG20 alginate is a relatively less viscous alginate than SL100 alginate. Results suggest that higher concentrations of alginate are better for needle formation whereas the 3% w/v SLG100 alginate formed the best microneedles. 2% w/v SLG100 also formed microneedles; however, they swelled once they were removed from the mold.

FIG. 10 is a graph of IL4 production after red light exposure. The results demonstrate sustained release from light triggered cells. Cells were seeded in a 96-well plate at a cell density of 30,000 cells per well. After a media change, cells were left in the dark for 24 hours and media was subsequently replaced. New media was added, and the same cells were then exposed to red light for the indicated time on the x-axis. At the indicated time, media was harvested immediately from 3 wells and stored at 4° C. until the ELISA was performed. (N=3)

FIG. 11 are images of PDMS microneedle patches. To ensure the PDMS did not adhere to the mold, molds were heated to 65° C. overnight prior to PDMS pouring. The exterior of the needles formed properly; in this embodiment, the base of the needle was the only element of the microneedle which was partially hollow after the molding process.

FIG. 12 is a collection of images illustrating a mouse wound model proof-of-concept. The drawing shows that two wounds are cut into the back of a mouse, one for treatment and one for control. Then, the wounds are splinted with silicone splints to ensure that the wounds do not contract after formation.

FIG. 13 is a graph showing fabrication of porous PDMS. To make PDMS patches that can house cells, the PDMS needs to be made porous and filled with alginate hydrogel. To fabricate porous PDMS, salt is added to PDMS while curing and dissolved afterward. Percent hollow volume was determined to the amount of alginate in porous PDMS.

FIGS. 14A-B demonstrate that hollow-tipped PDMS needles filled with cell-laden alginate allow for high-cell viability and structural stability of the needles. Epithelial cells were loaded into hollow-tipped PDMS needle patches and viability of the cells were determined with LIVE/DEAD staining after 24 hours. To fabricate the cell-laden porous PDMS, salt is added to PDMS while curing and dissolved afterward. Epithelial cells were suspended in alginate and crosslinked on the tips of the hollow PDMS needles. FIG. 14A shows stereoscope images of the needle tips with cells. FIG. 14B shows LIVE DEAD staining of the needle tips 24 hours post-fabrication.

FIGS. 15A-B demonstrate that engineered cells suspended in a patch can deliver factor to wound bed within 15 minutes after application. IL10 producing and sham (non-engineered) cells were separately suspended in alginate-tipped PDMS needle patches and placed in a murine wound for 15 minutes. LIVE/DEAD staining of patches before and after application shows that >90% cell viability was achieved in the patches. ELISAs of homogenized wound samples show that the patches with engineered cells delivered IL10 to the wound bed at the amounts shown indicated. “sham” denotes non-engineered cells. FIG. 15A depicts fluorescence microscopy imaging results of the cell viability/cytotoxicity assay. The needle tips were stained with LIVE/DEAD after 15 minutes in mouse wound. FIG. 15B is graph of ELISA data of the homogenized wound tissue with insert image of the patch on a wound bed.

FIG. 16 describes the design of the PDMS-alginate patch. Molds are 3D printed to shape PDMS during polymerization. In some embodiments of the patch, alginate (dyed blue here in the drawing) is crosslinked to the outside of the patch's pillars. In yet other embodiments of the patch, alginate is crosslinked inside a channel in the PDMS, which leads to more stability of the alginate in the patch.

FIGS. 17A-E illustrate an alternative patch fabrication method, resulting in increased stability of the alginate components of needle patches. FIG. 17A gives a schematic of the improved patch fabrication protocol. To mitigate the shearing of cell-laden alginate needle tips shear in response to moderate mechanical strain, the alternative fabrication method produces a porous and supportive membrane around the alginate tips. FIG. 17B shows a CAD model of designed top mold that was used in the improved fabrication method. FIG. 17C depicts the demolding and alginate loading steps to form alginate loaded PDMS needles. From the new molds, PDMS needles were fabricated with a channel in them that can be injected with alginate (dyed blue) and crosslinked. FIG. 17D depicts images of the needle tips loaded with cell-laden alginate employing the original and alternative fabrication methods. The alternative patch fabrication method creates a more stable needle head, so that the alginate does not shear off in the presence of mechanical strain. Shown is a comparison of the stability of patch emobodiments (fabricated using the two methods) after being vortexed for the indicated time. Alginate is dyed blue and is shown to shear off in the original design within 30 seconds, while the alternative design can withstand at least 10 minutes of vortexing before the alginate is sheared away into the wound bed FIG. 17E depicts fluorescence microscopy images of cell viability assays employing LIVE/DEAD stain. The alternative fabrication method can be loaded with cells, and the cells are still alive 24 hours later. In order to enhance the imaging of alginate inside the PDMS needles, the inner alginate was pulled out for better LIVE/DEAD staining and imaging of the cells within the yellow circle depicted in the drawing. Images were taken 24 hours after fabrication. Cells were quantified to be 99% viable.

FIGS. 18A-B illustrate that cells loaded into a hollow-cored PDMS needle patch are viable in a wound bed for up to a week in a murine model. FIG. 18A shows images of the wound dressing after wound formation and application of the patch to the wound site. In order to ensure that the patches remain in the wound, the wounds with the patches are covered in Tegaderm™ and the mouse was wrapped in Vetwrap™. Additionally, the Tegaderm™ allows the patches to remain wet, most likely contributing to the higher viability seen here as compared with previous canine studies. FIG. 18B shows a schematic of the patch in a murine wound and the results of the cell viability/cytotoxicity assay. Cells can be delivered into the wound bed employing the patch and survive for up to a week. Shown is LIVE/DEAD staining of full alginate patches and PDMS patches with alginate inserts after 3 days and 7 days in the wound bed. At day 3 both conditions have high cell viability (full alginate at 92% cells viable, PDMS-alginate patch at 97% cells viable). At day 7, the full alginate patches have 70% cell viability and the PDMS patches with alginate inserts have 51% cell viability, which indicates that both methods are viable in delivering cells to the wound bed.

FIG. 19 demonstrates that cytokine delivery modified the cell types of immune cells in the murine wound. Shown are UMAP plots of cells extracted from the wound bed with the indicated cytokines delivered from the tested patches. Each plotted point is a cell, grouped by gene expression similarities, allowing for identification of cell types. Listed percentages are the percent of total cells in that condition. Volcano plots of transcript-level differences compared to the control patch demonstrate that delivering IL10, TNF-α, and IL12 to the wound bed from the patches modifies the transcriptomes of macrophages in the wound bed.

FIG. 20 provides support that IL10 and IL12 secreting patches accelerate murine wound closure. Wounds were created on the backs of mice, and patches containing engineered cells were applied. Of the conditions tested, IL10 and IL12 secreting patches were shown to accelerate wound healing. Histology indicates that IL10 additionally reduces cell density in the wound bed, an indicator of scaring.

FIG. 21 show images of an LED panel element mounted to the back face of a PDMS patch containing optogenetically engineered cells. Dark media was harvested after the patches were left in the dark for 24 hours, and light media was harvested after the same patches were exposed to light for 24 hours. Also shown is a graph of light-activated IL-10 secretion (given in pg/mg tissue) from the assembled patch with mounted LED panel.

FIG. 22 shows images of an in vivo murine wound model with co-stimulation with IL10 and electrical pulses from the same patch, accompanied by a graph of IL10 secretion given in pg/mg tissue with electrical stimulation in the presence and absence of the patch. PDMS patches loaded with alginate-laden cells and electrical stimulators laminated on the surface were applied to a murine wound bed. ELISA data and electrical readings showed that IL10 was delivered concomitantly with electrical stimulation.

FIGS. 23A-E show a schematic of a patch.

FIG. 24 shows a schematic of the patch fabrication process.

FIG. 25 shows in vitro data giving the quantity of cells/pillar as a function of cell density in alginate hydrogel, as well as the production rate from the cells depending on the cell density in the alginate hydrogel.

FIGS. 26A-D are a schematic of the four-by-four PDMS patch and the transfer process of integrated array device on PDMS patch. FIG. 26A shows the molding the PDMS patch with two 3D printed molds. FIG. 26B illustrates the demolding of the PDMS patch. FIG. 26C is a schematic of the integrated array before lamination onto PDMS patch. FIG. 26D is a depiction of the integrated array device after lamination onto PDMS patch.

FIGS. 27A-B show the synthesis high-density electrode array for actuation and recording. FIG. 27A shows an optical image of a representative high-density flexible Au microelectrode array transferred on to a glass slide. FIG. 27B shows impedance at 1 KHz of 1 mm and 0.2 mm Au and PEDOT:PSS electrodes. Results are presented as mean±standard deviation (n=22-31 electrodes).

FIGS. 28A-D show the Electrochemical characterization of PEDOT:PSS electrodeposited electrodes. FIG. 28A shows impedance of flexible PEDOT:PSS electrodes with diameters of 20, 50, 100, and 200 μm. FIG. 28B shows Impedance at 1 KHz of flexible PEDOT:PSS electrodes. FIG. 28C shows Charge injection capacity of flexible PEDOT:PSS electrodes. FIG. 28D shows cathodal charge storage capacity of flexible PEDOT:PSS electrodes. Results presented as mean±standard deviation (n=5-9). Dark blue, orange, yellow, and purple represent 20, 50, 100, and 200 μm PEDOT:PSS electrodes.

FIGS. 29A-D show the stimulation of a rat sciatic nerve with a custom flexible stimulation array FIG. 29A shows sciatic nerve stimulation in an in vivo rat model with a custom PEDOT:PSS electrode array FIG. 29B shows Evoked skeletal muscle EMG activity under stimulation pulse of 250 μA amplitude and 200 us width using a 200 μm PEDOT:PSS electrode. FIG. 29C-D shows Muscle recruitment curves, illustrating peak-to-peak potential measured at different stimulation currents for using (C) 50 μm and (D) 200 μm. (n=10 per amplitude).

FIGS. 30A-E show Mapping VML wound bed electrophysiology. FIG. 30A shows Four 32-channel high density PEDOT:PSS coated MEAs (HD-MEAs) multiplexed on the wound bed of a canine. Average evoked EMG responses recorded using (FIG. 30B) 200 μm electrodes were used to produce muscle recruitment curves as shown in FIG. 30C. Average evoked EMG responses recorded using (FIG. 30D) 1 mm electroes were used to produce (FIG. 30E) muscle recruitment curves. Recruitment plot results are presented as mean±SD (n=15 for each stimulation current).

FIG. 31 shows Mapping muscle recruitment curve using high-density electrode arrays. Muscle recruitment map denoting the peak-to-peak potentials of EMG evoked under increasing current amplitudes at the COTS sciatic nerve stimulator. Map generated for array B presented in FIG. 29A. Linear interpolation was used to estimate the potential of non-functional electrodes. Results are presented as mean (n=15 for each stimulation current).

FIGS. 32A-C show high density evoked CMAP recording from canine VML wound model. (FIG. 32A) Canine VML wound with two proximal stimulation arrays and two distal recording arrays (highlighted in white). (FIG. 32B) Evoked recruitment curve Vₚₚ spatially mapped from 61 out of 64 recording electrodes. Recruitment curve points are the average Vₚₚ of 10 independent 0.1 ms pulses. The plotted evoked response threshold at ˜100 V matches the visual contraction threshold during stimulation. (FIG. 32C) Three dimensional spatially correlated Vₚₚ map. The more distal recording array displays a larger averaged Vₚₚ post threshold stimulation pulses relative the more proximal recording array. This may be indicative of more innervated skeletal muscle in the far distal region compared to the more proximal region. The three non-functional electrodes Vₚₚ values were interpolated in this plot.

FIGS. 33A-B show Patch fabrication for electrical and bio-chemical actuator integration. (FIG. 33A) PDMS patch with electrodes attached to tip. Cell laden alginate can be injected into a channel behind the electrodes. (FIG. 33B) Patch being applied to a mouse wound. The device is easily wrapped for security, leaving the leads sticking out of the bandage so the device can be turned on without removing the wrapping.

FIGS. 34A-C show High surface area three-dimensional fuzzy graphene (3DFG) electrodes. (FIG. 34A) Fabrication flow to test graphene-based materials in Cohen-Karni lab (3DFG or NT-3DFG). (FIG. 34B.I) The geometric size of the 3DFG electrodes as well as the density and size of the flakes can be modulated. Electrodes size is varied from 200 μm down to 20 μm (FIG. 34B.II). Inset of FIG. 34B.II shows scanning electron microscope image of the structure of 3DFG. FIG. 34C. Impedance at 1 KHz of Pt and 3DFG microelectrodes as a function of size. Results are mean±SD (n=6).

FIGS. 35A-F show Electro-polymerization of Ni-TMHPP on 3DFG MEAs. (FIG. 35A) Representative cyclic voltammogram for cyclic voltammetry based electro-polymerization of Ni-TMHPP on 3DFG MEAs at a scan rate of 50 mV/s. (FIG. 35B) (I) Bright field image of a representative 200 μm 3DFG MEA after electro-polymerization of Ni-TMHPP. (II) Raman spectra acquired from Ni-TMHPP drop casted on to a 600 nm Si/SiO₂ substrate (black) and Ni-TMHPP electro-polymerized on 3DFG MEA (orange, yellow, purple, green, blue, and red as marked by x in (I)). (FIG. 35C) Representative cyclic voltammogram of 3DFG MEA before and after electro-polymerization of Ni-TMHPP acquired at a scan rate of 100 mV/s. (FIGS. 35D-F) (I) Electrode impedance and (I) negative phase of 3DFG MEAs before and after electro-polymerization of Ni-TMHPP. Results are presented as mean±SD (n=3 electrodes).

FIGS. 36A-D show Electrochemical sensing of NO via square wave voltammetry method. (FIG. 36A) Comparison of SWV response of 3DFG and Pt 200 μm microelectrodes to 181.2 μM NO in PBS solution, demonstrating pronounced response of 3DFG to NO over standard metallic electrodes. (FIG. 36B) SWV response of 3DFG microelectrodes modified with Iron phthalocyanine (FePc) to different concentrations of NO in PBS solution. (FIG. 36C) SWV response of 3DFG microelectrodes modified with Nickel (II) tetrakis(3-methoxy-4-hydroxyphenyl) porphyrin (Ni-TMHPP) to different concentrations of NO in PBS solution. (FIG. 36D) Comparison of the NO sensitivities of various sensing materials and surface coatings (data shown as mean±SD, n=3).

FIGS. 37A-D show Electrochemical sensing of NO via chronoamperometry method. (FIG. 37A) Current response of 200 μm 3DFG microelectrode sensor to aliquots of stock NO-saturated PBS solution (representative, n=3). (FIG. 37B) Calibration curve for sensor measurements performed in FIG. 37A. NO sensitivity of 0.216±0.01 nA uM⁻¹ and limit of detection (LOD) of 40.2±1.9 nM. (FIG. 37C) Current response of 200 μm 3DFG microelectrode sensor to stock NO-saturated PBS solution added via syringe pump to reduce current spiking. (FIG. 37D) Calibration curve for sensor measurements performed in FIG. 37C. NO sensitivity of 0.244±0.05 nA uM⁻¹ and limit of detection (LOD) of 11.1±2.5 nM.

FIGS. 38A-D show semipermeable Nafion coatings for selective electrochemical sensing of NO. (FIG. 38A) Profilometric layer thickness of various Nafion casting methods. Spin casting was performed at 2500 rpm. (FIG. 38B) Interferent screening properties of thick Nafion layers (5 wt. % drop cast) on a 3DFG sensor. Representative calibrations, n=3, were performed with FePc 3DFG WE, Pt CE, and Ag/AgCl RE in N₂-saturated PBS. (FIG. 38C) NO sensing calibration 3DFG sensor with a thin Nafion layer (5 wt. % spin cast). (FIG. 38D) Summary plot of the sensitivity of the sensors to NO and interferent, NO₂⁻, before and after Nafion casting with data given as mean±std, n=3.

FIGS. 39A-F show Electrochemical modification and characterization of planar platinum electrodes. (FIG. 39A) Representative cyclic voltammogram of platinum electrode for 5 cycles acquired with 1×PBS at a scan rate of 100 mV/s. (FIG. 39B) Representative electropolymerization of 5AN1 by 5 cycles of cyclic voltammetry acquired with 10 mM 5AN1 in 1×PBS adjusted to pH=1 by HCl at scan rate of 10 m V/s. (FIG. 39C) Representative Raman spectroscopy of 5AN1 and fluorinated xerogel coated on planar platinum electrodes. Inset: Image of 5AN1 modified sensor. (FIG. 39D) Representative electrochemical Impedance spectroscopy characterization of surface modified electrodes acquired with 1×PBS. (FIG. 39E) Representative cross-sectional SEM of 5AN1 deposited on planar platinum electrode. (FIG. 39F) Representative cross-sectional SEM of spray coated fluorinated xerogel.

FIGS. 40A-H show Platinum black modification and characterization. (FIG. 40A) Representative cyclic voltammetry characterization of platinum black electrodes at different deposition charge densities acquired with 1×PBS at scan rate of 100 mV/s. Inset: image of platinum black electrodes. (FIG. 40B) Quantification of charge storage capacity of platinum black electrode deposited at different charge densities. (FIG. 40C) Representative SEM image of platinum black deposited at 25 mM H2PtCl6, −0.3V reduction potential and 25 nC/μm2 deposition charge density. (FIG. 40D) Representative electrochemical impedance spectra of platinum black deposited at different charge densities acquired with 1×PBS. (FIG. 40E) Quantification of impedance at 1 Hz for platinum black deposited at different charge densities. (FIG. 40F) Representative cyclic voltammogram for 5AN1 electropolymerization on planar and nanostructured platinum acquired with 10 mM 5AN1 in 1×PBS adjusted to pH=1 by HCl. (FIG. 40G) Representative EIS spectra comparison pre and post 5AN1 electropolymerization on nanostructured platinum electrode acquired with 1×PBS. (FIG. 40H) Quantification of impedance at 1 Hz for platinum black and 5AN1 electropolymerized platinum black electrodes.

FIGS. 41A-F show semipermeable electrodeposited coatings for selective electrochemical sensing of NO. NO sensor calibration before (FIGS. 41A-B) and after (FIGS. 41C-D) electrodeposition of selective polymer layers, Eugenol and 5-amino-1-naphthol (5AN1). Representative calibrations, n=3, were performed with FePc 3DFG WE, Pt CE, and Ag/AgCl RE in N₂-saturated PBS. (FIG. 41B) Calibration curve for data shown in FIG. 41A. (FIGS. 41E-F) Summary plot of the sensitivity of the sensors to NO (FIG. 41E) and interferent, NO₂, (FIG. 41F) before and after electrodeposition with data given as mean±std, n=3.

FIGS. 42A-F show In vitro NO sensing calibration in the controlled setup (FIG. 42A) Representative image of calibration setup; (FIG. 42B) Representative 8-channel multiplexed chronoamperometry calibration in 1×PBS and in response to NO solutions and interferent solutions of nitrite, ascorbic acid and uric acid; Inset: Representative top-down view of flexible sensing array; (FIG. 42C) Representative linear regression of current reading to cumulative nitric oxide concentration; Inset: Representative image of flexible sensor in hand; (FIG. 42D) Sensitivity quantification for planar Pt-5AN1-Xero, planar Pt-5AN1 and NSPt-5AN1. *: p<0.05 for one-way ANOVA statistical analysis; (FIG. 42E) Quantification of limit of detection for planar Pt-5AN1-Xero, planar Pt-5AN1 and NSPt-5AN1; (FIG. 42F) Quantification of selectivity for planar Pt-5AN1-Xero, planar Pt-5AN1 and NSPt-5AN1. Data was collected as n=16 for Planar Pt-5AN1-Xero, n=16 for Planar Pt-5AN1 and n=5 for NSPt-5AN1.

FIGS. 43A-D show In vitro NO sensing experiments with RAW macrophages. (FIG. 43A) Optical image of RAW macrophages seeded onto the surface of the NO sensor at the 3DFG working electrode (WE). Inset: Image of prototype MEA chip on printed circuit board for in vitro experiments with 2 mL of cell media and 10⁶ RAW macrophages seeded into the well. (FIG. 43B) Continuous current recordings from 3DFG WE after macrophages are polarized with IFN-γ and LPS at time=0. (FIG. 43C) Griess assay measurements for nitrite concentration from polarized macrophages as a function of time. Data given as mean±std, n=3. (FIG. 43D) Continuous current recordings from WPI Inc. NO probe after macrophages are polarized with INF-γ and LPS at time=0.

FIGS. 44A-H show multiplexed electrochemical NO detection from 8 sensors (FIGS. 44A-G) NO sensor calibrations performed with FePc 3DFG (Channel 1, 3, and 8) or poly(Eug) 3DFG (Channel 4, 5, and 6) WEs, Pt CE, and Ag/AgCl RE in N₂-saturated PBS. Recordings taken via alternating current recordings from each channel (8 s total sampling period). (FIG. 44H) Background-subtracted overlay of data shown in FIGS. 44A-G.

FIGS. 45A-D show poly(5AN1) Coatings on Pt Macroelectrode Arrays. (FIG. 45A) Optical image of Pt 3-mm and 0.5-mm electrodes prior to electrodeposition of poly(5-amino-napthol-1) (5AN1). (FIGS. 45B & 45C) Representative optical image of poly(5AN1) electropolymerized with 5 and 20 cyclic voltammogram (CV) cycles, on 3-mm Pt electrode, n=3. (FIG. 45D) Representative Raman spectra of Pt electrodes before (pristine) and after depositing poly(5AN1) with 5 and 20 cycles, with Raman peak positions indicating the presence of poly(5AN1), n=3. *After washing Pt electrodes that were not electrodeposited, some Raman signal could still be detected, which may indicate physiosorbed 5AN1 monomer on the electrode surface.

FIGS. 46A-D show the sensitive and selective NO detection after coating Pt with poly(5AN1). NO selectivity against interferents nitrite (NO₂⁻), ascorbic acid (AA), and uric acid (UA) before (FIG. 46A) and after (FIG. 46C) electrodeposition of selective polymer layers, poly(5AN1). Representative calibrations, n=3, were performed with Pt 3-mm dia. WE, Pt CE, and Ag/AgCl RE in N₂-saturated PBS. (FIG. 46B) Representative CV curve for the deposition of 5AN1 on Pt 3-mm electrodes performed from 0.3 to 1 V vs Ag/AgCl at 10 mV s⁻¹ for 5 cycles. (FIG. 46D) Summary graph of the selectivity of the sensors to NO versus interferents, before and after electrodeposition with data given as mean±std, n=3.

FIGS. 47A-B. Low NO detection limit of poly(5AN1) 3DFG electrodes. (FIG. 47A) NO sensor calibration after electrodeposition of poly(5AN1). Representative calibrations, n=2, were performed with 3DFG WE, Pt CE, and Ag/AgCl RE in N₂-saturated PBS. (FIG. 47B) Calibration curve for data shown in FIG. 47A.

FIGS. 48A-G show Standardization and comparison with commercial NO probe. (FIG. 48A) Concentration of NO released by DEA NONOate salt in PBS buffer and measured with respect to time by ISO NOP sensor. (FIG. 48B) Corresponding resonance shift due to NO release with respect to time measured with fabricated SOI device. (FIGS. 48C-D) Low concentration NO detection by SOI device from NO bubbling system. Resonance shift for low concentration is very small. The device could measure minimum 10 μM concentration with confidence. (FIGS. 48E-G) Resonance shift due to interferent's are smaller (<40 pm for up to 300 μM conc.) which shows good selectivity.

FIGS. 49A-D show flexible Sensor Array Fabrication and Characterization. (FIG. 49A) Wafer-scaled fabrication of flexible sensor arrays on Si/600 nm SiO₂ substrate before releasing. (FIG. 49B) Representative released flexible sensor array on glass slide. (FIG. 49C) Image of a representative flexible sensor array including different size Pt electrodes (0.5 mm, 1 mm, and 3 mm) and Au reference electrode (REF)+counter electrode (CE) (FIG. 49D) EIS of representative 0.5 mm, 1 mm, and 3 mm Pt electrodes (n=3).

FIGS. 50A-B show flexible NO Sensor Array Ex vivo Demonstration. (FIG. 50A) Demonstration of Ex vivo experimental set-up on chicken breast. (FIG. 50B) Representative calibration curve of flexible sensor array responding of NO aliquots (DEA-NONOate) and 1×PBS aliquot. The 1×PBS aliquots was added to benchmark the interference level during adding solution.

FIGS. 51A-F show flexible NO Sensor Array Ex vivo Rodent Wound Model. (FIG. 51A) Image of wound surface on ex vivo control rodent without (I) and with (II) placement of the flexible NO sensor array. (FIG. 51B) Sensor current response from control rat for 1 h. (FIG. 51C) Inset for FIG. 51B showing response of sensor to 100 μL aliquots of 1×PBS and 10 mM L-Arg. (FIG. 51D) Image of wound surface on ex vivo rodent with inflammatory polypropylene mesh implant without (I) and with (II) placement of the flexible NO sensor array. (FIG. 51E) Sensor current response from inflammatory mesh for 1 h. (FIG. 51F) Inset for FIG. 51E showing response of sensor to 100 μL aliquot 10 mM L-Arg.

FIGS. 52A-D show In vivo Canine NO Sensing. (FIG. 52A) NO sensor array placement on VML injury model (canine) at 0 h after VML injury. (FIG. 52B) Multiplexed 3 sensor recording from the VML injury model for 10 min. (FIG. 52C) NO sensor array placement on VML injury model (canine) at 24 h after VML injury. (FIG. 52D) Multiplexed 2 sensor recording from the VML injury model for 10 min.

FIGS. 53A-E show in vivo Canine NO Sensing. (FIG. 53A) NO sensor array placement on VML injury model (canine) wound center. (FIG. 53B) NO sensor array placement on canine wound edge. (FIG. 53C) Current reading from NO sensor. (FIG. 53D) Baseline NO reading over the 14-day post wound surgery. (FIG. 53E) Gene expression analysis from biopsy samples at locations where NO sensors were placed.

FIGS. 54A-B show low form factor NO sensor array and backend connections. (FIG. 54A) Optical image of 1 mm Pt electrode array. (FIG. 54B.I) Handling of flexible NO sensor array with ACF cable and polyimide tape to seal bond pad sensor. (FIG. 54B.II) Packaging of flexible NO sensor array for in vivo measurement. The ACF cable bonds to a small custom printed circuit board with pin headers (or Molex Pico Ezmate) connectors to shielded electrical cabling. The flexible sensor array is placed on a 1″ glass slide until removal and placement in vivo.

FIGS. 55A-F show coating, selectivity, and sensitivity of Xerogel 5AN1-Au-based NO sensors. (FIG. 55A) Xerogel film thickness as a function of spray coating time. Spray coating performed at 17 cm away from sample at 25 psi. (FIGS. 55B-C) Optical image of 5AN1-Au sensor array before (B) and after (C) xerogel spray coating for 7 s as described in FIG. 55A. (FIGS. 55D-F) Electrical impedance spectroscopy of 5AN1-Au sensor array before (D) and after (E) xerogel spray coating for 7 s as described in FIG. 55A. The results are presented as mean±standard deviation (n=6). (FIG. 55F) Calibration curves (n=3) of 1 mm Xerogel-5AN1-Au sensor array in 1×PBS with 3 aliquots of interference [NO₂⁻, uric acid (UA), ascorbic acid (AA)] and 1 aliquot of NO (DEA-NONOate).

FIGS. 56A-F show stability of NO sensor under physiological conditions. (FIGS. 56A-C) Calibration curves of 1 mm Pt-5AN1 sensors with 3 aliquots of interference (NO₂″, uric acid (UA), ascorbic acid (AA)) and 1 aliquot of NO (DEA-NONOate). (A) Performance of sensor in 1×PBS before incubating in media (S_{NO}=11.7±1.0 nA μM⁻¹, n=5). (B) Performance of sensor in serum-contained media (S_{NO}=1.29±0.03 nA μM⁻¹, n=3). The sensors were incubated in complete media for 24 hours at room temperature. The media composition is Dulbecco's Modified Eagle Medium (DMEM)+10% Fetal Bovine Serum (FBS)+1% penicillin-streptomycin+1% Glutamax. (C) Performance of sensor incubated in media for 24 hours, washed with DI water, and tested in 1×PBS (S_{NO}=8.8±0.6 nA μM⁻¹, n=4). (FIGS. 56D-F) Selectivity of NO₂, AA and UA in the test conditions same in FIGS. 56A-C. All results are presented as mean±standard deviation.

FIGS. 57A-D show patterned pseudo-reference ink-based electrode. (FIG. 57A) Au electrode coated with Ag/AgCl ink for electrochemical characterizations in FIG. 57B & FIG. 57C. (FIG. 57B) Cyclic voltammetry of glassy carbon in 1 mM [Fe(CN)₆]³⁻. Potential versus commercial REF shown in red, versus pseudo-REF/CE shown in blue. (FIG. 57C) Open circuit potential between commercial Ag/AgCl REF and pseudo-REF/CE with a potential drift over 12 hours of 0.001 mV/min. (FIG. 57D) Stereoscope image of microbrush (Nicpro Flat 2) painted pseudo-REF/CE onto a glass slide using a 1 mil PET film stencil, cut using LPKF Protolaser. The glass slide was heated to 80° C., Ag/AgCl ink was painted over each individual cut-out from bottom to top of design, cured for 10 min at 110° C., and the stencil was removed post-cure. The average thickness of the Ag/AgCl coating was 59.9±12.0 μm.

FIGS. 58A-F show selectivity and sensitivity of 5AN1-Au-based sensors. (FIGS. 58A-C) Representative calibration curve (n=4) of 5AN1-Au sensors with diameter of 3 mm (FIG. 58A), 1 mm (FIG. 58B), and 0.5 mm (FIG. 58C) in 1×PBS with 3 aliquots of interferents (NO₂⁻, UA, and AA) and 1 aliquot of NO (DEA NONOate). (FIGS. 58D-F) NO selectivity of 5AN1-Au sensors with diameter of 3 mm (FIG. 58D), 1 mm (FIG. 58E), and 0.5 mm (FIG. 58F) against NO₂⁻, AA, and UA under the test conditions shown in FIGS. 58A-C on logarithmic scale. All results are presented as mean±standard deviation.

FIGS. 59A-F show in vivo rodent acute NO sensing. (FIG. 59A) Image of NO sensor array placement on VML injury model (rodent) at day 5 after surgery. (FIG. 59B) Multiplexed three 1 mm dia. sensors recording from the VML injury model for ˜1 hour at day 5. (FIG. 59C) Inset view of recording with aliquots of L-Arginine and 1×PBS. (FIG. 59D) L-Arginine induced NO production at (FIG. 59D) day 5, (FIG. 59E) day 7, (FIG. 59F) day 14. Baseline subtraction of recorded current was performed with 8-points exponential function fitting and the NO concentrations were converted with NO sensitivity of each sensor.

FIGS. 60A-F show the rat wound model in vivo NO sensing experiment. (FIG. 60A) Representative image of flexible sensor placement on rat wound. (FIG. 60B) Representative raw data of chronoamperometry current reading on wound for 1 hour NO sensing; Inset: Zoomed in chronoamperometry current recording with sensor response verified by stimulus of L-arg and control of PBS. (FIG. 60C) Temporal measurement of baseline NO concentration on the wound. (FIG. 60D) Representative spatial measurement of baseline nitric oxide concentration on wound. (FIG. 60E) Temporal measurement of stimulated NO concentration. (FIG. 60F) Representative spatial measurement of L-arg induced nitric oxide concentration on wound.

FIG. 61 shows the rat wound model in vivo experiment design, including timeline of surgery and NO sensing.

FIGS. 62A-B shows NO induced by L-arginine as a function of post-surgery time. (A) L-Arginine induced NO production over different post-surgery time. (B) L-Arginine induced NO production.

FIGS. 63A-C show NO sensor array performance before and after in vivo. The NO sensor array response (FIG. 63A) before and (FIG. 63B) after 1 h recording on rodent model. (FIG. 63C) (I) NO sensitivity both before and after in vivo experiment. n.s. indicates no significant difference (one-way ANOVA and post-hoc Tukey) (II) NO selectivity with nitrite, ascorbic acid (AA), and uric acid (UA) both before and after in vivo experiment. n.s. indicates no significant difference (one-way ANOVA and post-hoc Tukey).

FIGS. 64A-C show a high-density sensor array design for multiplexing and dual sensing. New sensor array design fabricated with (FIGS. 64A-B) 16 channels with 2 different reference/counter electrode configuration and with (FIG. 64C) 8 channels.

### DETAILED DESCRIPTION

Although the wound bed is known to be dynamic and heterogenous, most current treatments for wound healing are static and homogeneous such as Apligraf®, a wound healing treatment that is composed of Matrigel with dermal fibroblasts and epidermal keratinocytes. This limits the effectiveness of such therapies and does not address particularly challenging clinical situations such as chronic/non-healing wounds.

The present disclosure describes a unique treatment approach comprising a patch that has controllable release of wound healing factors with optogenetically engineered cells. Since the light applied to a wound can be controlled, this allows for different wound healing factors to be delivered to different parts of the wound bed and at different times. This also allows for wound healing factors to be delivered over longer time periods in an ongoing and dynamic fashion much like the natural progression of wound healing.

In addition, the devices may include a biosensor to monitor healing, such as nitric oxide (NO) or TGF-β1. Current NO sensors have limitations such as high limit of detection, low selectivity, low stability over time and single detected point. Thus, the inventors have developed a micro-fabricated NO sensor arrays with high sensitivity and selectivity, which can achieve the multi-point NO mapping on the wound and direct the factor release. Therapeutic electrical stimulation directly to a wound is also proposed as s a further therapy provided by the device. These and other aspects of the disclosure are described in detail below Definitions.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number. “Heal” as used herein refers to the partial or complete restoration of a cell or tissue containing a wound, e.g., a wound described herein,

The terms “subject” or “patient,” as used herein, refer to an individual bearing a wound and/or the recipient of a wound healing device described herein. The subject may include a human (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of, e.g., a wound, e.g., as described herein. Treating may entail administering or applying the wound healing device described herein. In an embodiment, treating comprises reducing, reversing, alleviating, delaying the onset of, or inhibiting the worsening of a wound in a subject. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. In some embodiments, treatment comprises prevention and in other embodiments it does not.

The term “wound,” as used herein, refers to any disruption, from whatever cause, of the skin or underlying tissue. Exemplary wounds include, e.g., abrasions, surgical incisions, cuts, punctures, tears, sores, ulcers, blisters, burns, amputations, and bites. Exemplary disruptions include, e.g., inflamed areas, polyps, and ulcers. Underlying tissues include those tissues not normally exposed in the absence of a wound or disruption, such as, e.g., muscle or connective tissue. A wound is not necessarily visible, nor does it necessarily involve rupture of superficial tissue. A wound may be further described as an open wound or a closed wound. An “open wound,” as used herein, refers to a break in the skin that leaves internal tissue exposed. Exemplary open wounds include, e.g., incisions, lacerations, abrasions, and avulsions. A “closed wound,” as used herein, refers to a wound where tissue damage or bleeding occurs below the surface of the skin. Exemplary closed wounds include, e.g., bruises, contusions, blisters, seromas, and hematomas.

### I. WOUNDS

### Classification of Wounds

A wound is a type of injury which may happen quickly and can result in a disruption, defect, or tear in the skin and/or underlying tissue. For example, the skin may be torn, cut, or punctured (an “open” wound) or where blunt force trauma causes a contusion (a “closed” wound). In pathology, it specifically refers to a sharp injury which damages the epidermis of the skin. According to level of contamination, a wound can be classified as:
Clean wound-made under sterile conditions where there are no organisms present, and the skin is likely to heal without complications.
Contaminated wound—usually resulting from accidental injury; there are pathogenic organisms and foreign bodies in the wound.
Infected wound—the wound has pathogenic organisms present and multiplying, exhibiting clinical signs of infection (yellow appearance, soreness, redness, oozing pus).
Colonized wound—a wound containing pathogenic organisms, difficult to heal (e.g., bedsore).
   Wounds can also be categorized as acute or chronic, with the latter presenting particular challenges in the healing process. In an embodiment, an acute wound refers to a wound that quickly heals. In an embodiment, chronic wounds that are not healing should be investigated to find the causes; many microbiological agents may be responsible. The basic workup includes evaluating the wound, its extent and severity. Cultures are usually obtained both from the wound site and blood. X-rays are obtained and a tetanus shot may be administered if there is any doubt about prior vaccination. Bacterial infection of wound is a potential issue for nearly all wounds and can impede the healing process, even leading to life-threatening complications.

Wound size is often the first and most important assessment of a wound. There are many methods known in the art for measuring the size of wounds (see, e.g., Int. Wound J. 2016 August; 13 (4): 540-553) A wound may be measured by, e.g., its linear dimensions (e.g., length and width). Exemplary methods of obtaining the dimensions of a wound include ruler-based methods, transparency tracings, and photography. In other instances, ascertaining the volume of a wound may be necessary. Exemplary methods of determining the volume of a wound include, e.g., ruler-based methods, casts (i.e., creating a mold), saline methods, stereophotogrammetry, and structured light readings (see, e.g., J. Wound Care 2009; 18 (6): 250-253).

The severity of a wound may be quantified by one or more wound assessment systems or scales known in the art. Exemplary wound assessments or scales include, e.g., Wagner System, Southampton Wound Scoring system, DEPA Scoring System, The University Of Texas System, Bates-Jensen Wound Assessment Tool TIME-H system, HEDI system, red-yellow-black-scheme (RYB) and others. The wound healing device described herein may be used to treat a wound of any size in a subject. A wound may be about, e.g., 0.1 mm, 0.5 mm, 0.75 mm, 1 mm, 1.5 mm, 2 mm, 2.5 mm, 3 mm, 4 mm, 5 mm, 6 mm, 7 mm, 8 mm, 9 mm, 10 mm, 12.5 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm, 200 mm, or 300 mm, e.g., in a longest linear dimension, e.g., length or width. In an embodiment, the wound is greater than 5 mm, 10 mm, 20 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm, 200 mm, or 300 mm, e.g., in a longest linear dimension, e.g., length or width. In an embodiment, the wound is less than 5 mm, 10 mm, 20 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm, 200 mm, or 300 mm, e.g., in a longest linear dimension, e.g., length or width. In an embodiment, the wound is greater than 5 mm, 10 mm, 20 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, 200 mm, or 300 mm, e.g., in a longest linear dimension, e.g., length or width.

For example, a wound may be about 0.1 cm, 0.5 cm, 0.75 cm, 1 cm, 1.5 cm, 2 cm, 2.5 cm, 3 cm, 4 cm, 5 cm, 6 cm, 7 cm, 8 cm, 9 cm, 10 cm, 12.5 cm, 15 cm, 20 cm, 25 cm, 30 cm, 35 cm, 40 cm, or 45 cm, e.g., in a longest linear dimension, e.g., length or width. In an embodiment, the wound is greater than 5 mm, 10 mm, 20 mm, 30 mm, 35 cm, 40 cm, or 45 cm, e.g., in a longest linear dimension, e.g., length or width. In an embodiment, the wound is less than 5 mm, 10 mm, 20 mm, 30 mm, 35 cm, 40 cm, or 45 cm, e.g., in a longest linear dimension, e.g., length or width.

The wound healing device described herein may be used to treat a wound of any depth in a subject. For example, a wound may be about 0.1 mm, 0.5 mm, 0.75 mm, 1 mm, 1.5 mm, 2 mm, 2.5 mm, 3 mm, 4 mm, 5 mm, 6 mm, 7 mm, 8 mm, 9 mm, 10 mm, 12.5 mm, 15 mm, 20 mm, 25 mm, 30 mm, 35 mm, 40 mm, 45 mm, 50 mm, 55 mm, 60 mm in depth. In an embodiment, a wound may greater than 0.1 mm, 0.5 mm, 0.75 mm, 1 mm, 1.5 mm, 2 mm, 2.5 mm, 3 mm, 4 mm, 5 mm, 6 mm, 7 mm, 8 mm, 9 mm, 10 mm, 12.5 mm, 15 mm, 20 mm, 25 mm, 30 mm, 35 mm, 40 mm, 45 mm, 50 mm, 55 mm, or 60 mm in depth. In an embodiment, the wound is less than 0.1 mm, 0.5 mm, 0.75 mm, 1 mm, 1.5 mm, 2 mm, 2.5 mm, 3 mm, 4 mm, 5 mm, 6 mm, 7 mm, 8 mm, 9 mm, 10 mm, 12.5 mm, 15 mm, 20 mm, 25 mm, 30 mm, 35 mm, 40 mm, 45 mm, 50 mm, 55 mm, or 60 mm in depth.

A wound may be about 0.1 cm, 0.2 cm, 0.3 cm, 0.4 cm, 0.5 cm, 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.5 cm, 3 cm, 4 cm, 5 cm, 6 cm, 7 or cm in depth. In an embodiment, a wound may be greater than about 0.1 cm, 0.2 cm, 0.3 cm, 0.4 cm, 0.5 cm, 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.5 cm, 3 cm, 4 cm, 5 cm, 6 cm, 7 or cm in depth. In an embodiment, the wound is less than about 0.1 cm, 0.2 cm, 0.3 cm, 0.4 cm, 0.5 cm, 0.6 cm, 0.7 cm, 0.8 cm, 0.9 cm, 1 cm, 1.1 cm, 1.2 cm, 1.3 cm, 1.4 cm, 1.5 cm, 1.6 cm, 1.7 cm, 1.8 cm, 1.9 cm, 2 cm, 2.5 cm, 3 cm, 4 cm, 5 cm, 6 cm, 7 or cm in depth. The wound healing device described herein may be used to treat or heal a wound of any shape. Wounds may be symmetrical (i.e., have one or more planes of symmetry) or be asymmetrical. For example, a wound may be circular, oblong, elliptical, oval-shaped, square, rectangular, or rod-shaped. A wound may have a complex or irregular shape.

The wound healing device described herein may be used to treat a wound comprising areas of necrotic tissue in a subject. The wound healing device described herein may be used to treat a wound comprising areas of eschar or induration (i.e., thick or hardened skin). In some embodiments, the wound comprises 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99% necrotic tissue. In some embodiments, the wound comprises greater than 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99% necrotic tissue. In some embodiments, the wound comprises less than 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99% necrotic tissue.

The wound healing device described herein may be used to treat a wound comprising a exudate, e.g., a seeping material from the wound. The extent of exudate may be described as heavy (e.g., wound dressing or bandage is soaked), medium, minimal, or absent.

### Wound Healing Processes

To heal a wound, the body undertakes a series of actions collectively known as the wound healing process. Actions taken by the medical professional or patient may greatly improve the healing process. The overall treatment depends on the type, cause, and depth of the wound, and whether other structures beyond the skin (dermis) are involved. Treatment of recent lacerations involves examining, cleaning, and closing the wound. Minor wounds, like bruises, will heal on their own, with skin discoloration usually disappearing in 1-2 weeks. Abrasions, which are wounds with intact skin (non-penetration through dermis to subcutaneous fat), usually require no active treatment except keeping the area clean, initially with soap and water. Puncture wounds may be prone to infection depending on the depth of penetration. The entry of puncture wound is left open to allow for bacteria or debris to be removed from inside.

Evidence to support the cleaning of wounds before closure is scant. For simple lacerations, cleaning can be accomplished using a number of different solutions, including tap water and sterile saline solution. Infection rates may be lower with the use of tap water in regions where water quality is high. Cleaning of a wound is also known as ‘wound toilet’. It is not clear if delaying a shower following a surgery helps reduce complications related to wound healing.

Evidence is insufficient to conclude whether cleaning wounds is beneficial or whether wound cleaning solutions (polyhexamethylene biguanide, aqueous oxygen peroxide, etc.) are better than sterile water or saline solutions to help venous leg ulcers heal. It is also uncertain whether the choice of cleaning solution or method of application makes any difference to venous leg ulcer healing. After this point in time, however, there is a theoretical concern of increased risks of infection if closed immediately. Thus, some healthcare providers may delay closure while others may be willing to immediately close up to 24 hours after the injury.

If closure of a wound is decided upon a number of techniques can be used. These include bandages, a cyanoacrylate glue, staples, and sutures. Absorbable sutures have the benefit over non absorbable sutures of not requiring removal. They are often preferred in children. Buffering the pH of lidocaine makes the injection less painful. Adhesive glue and sutures have comparable cosmetic outcomes for minor lacerations <5 cm in adults and children. The use of adhesive glue involves considerably less time for the doctor and less pain for the person. The wound opens at a slightly higher rate but there is less redness. The risk for infections (1.1%) is the same for both. Adhesive glue should not be used in areas of high tension or repetitive movements, such as joints or the posterior trunk. Split-thickness skin grafting (STSG) is also a surgical technique that features rapid wound closure, multiple possible donor sites with minimal morbidity.

In the case of clean surgical wounds, there is no evidence that the use of topical antibiotics reduces infection rates in comparison with non-antibiotic ointment or no ointment at all. Antibiotic ointments can irritate the skin, slow healing, and greatly increase the risk of developing contact dermatitis and antibiotic resistance. Because of this, they should only be used when a person shows signs of infection and not as a preventative.

### A. Acute Wounds

Acute wounds include both surgical and non-surgical wounds, and in some cases from disease. Acute wounds may transition into chronic wounds, which are discussed below. Surgical wounds are at least initially classified as traumatic wounds. Non-surgical wounds can be caused by disease but, in most instances, are also traumatic wounds. Types of non-surgical trauma include car/train/bus/motorcycle/bicycle/ATV accidents, gunshot wounds, concussions, knife wounds, construction site accidents, industrial accidents, explosions, crushing injuries, and a host of other events leading to acute injury.

Airway management, monitoring, and management of injuries are all key guidelines when it comes to medical trauma care. Airway management is a key component of emergency on-scene care. Using a systematic approach, first responders must access that a patient's airway is not blocked in order to ensure the patient gets enough circulation and remain as calm as they can. Monitoring patients and making sure their body does not go into shock is another essential guideline when it comes to medical trauma care. Nurses are required to watch over patients and check blood pressure, heart rate, etc. to make sure that patients are doing well and are not crashing. When it comes to managing injuries, head and neck injuries require the most care post-surgery. Head injuries are one of the major causes of trauma related death and disabilities worldwide. It is important for patients of head trauma to get CT scans post-surgery to ensure that there are no problems.

### B. Chronic Wounds

A chronic wound is a wound that may not not heal in an orderly set of stages and in a predictable amount of time; wounds that do not heal within three months are often considered chronic. Chronic wounds seem to be detained in one or more of the phases of wound healing. For example, chronic wounds often remain in the inflammatory stage. To overcome that stage and jump-start the healing process, a number of factors need to be addressed such as bacterial burden, necrotic tissue, and moisture balance of the whole wound. In acute wounds, there may be a balance between production and degradation of molecules such as collagen; in chronic wounds this balance is lost and degradation may play an outsized role. Chronic wounds may never heal or may take years to do so. These wounds may cause patients emotional and physical stress.

Acute and chronic wounds may be at opposite ends of a spectrum of wound-healing types that progress toward being healed at different rates. Chronic wound patients often report pain as dominant in their lives. It is recommended that healthcare providers handle the pain related to chronic wounds as a priority in chronic wound management (together with addressing the cause). Six out of ten venous leg ulcer patients experience pain with their ulcer, and similar trends are observed for other chronic wounds. Persistent pain (at night, at rest, and with activity) is the main problem for patients with chronic ulcers. Frustrations regarding ineffective analgesics and plans of care that they were unable to adhere to were also identified.

In addition to poor circulation, neuropathy, and difficulty moving, factors that contribute to chronic wounds include systemic illnesses, age, and repeated trauma. The genetic skin disorders collectively known as epidermolysis bullosa display skin fragility and a tendency to develop chronic, non-healing wounds. Comorbid ailments that may contribute to the formation of chronic wounds include vasculitis (an inflammation of blood vessels), immune suppression, pyoderma gangrenosum, and diseases that cause ischemia. Immune suppression can be caused by illnesses or medical drugs used over a long period, for example steroids. Emotional stress can also negatively affect the healing of a wound, possibly by raising blood pressure and levels of cortisol, which lowers immunity.

Another factor that may contribute to chronic wounds is age. The skin of older people may be more easily damaged, and older cells may not proliferate as fast and may not exhibit an adequate response to stress in terms of gene upregulation of stress-related proteins. In older cells, stress response genes may be overexpressed when the cell is not stressed, but when it is, the expression of these proteins is not upregulated by as much as in younger cells.

Comorbid factors that can lead to ischemia are especially likely to contribute to chronic wounds. Such factors include chronic fibrosis, edema, sickle cell disease, and peripheral artery disease such as by atherosclerosis.

Repeated physical trauma plays a role in chronic wound formation by continually initiating the inflammatory cascade. The trauma may occur by accident, for example when a leg is repeatedly bumped against a wheelchair rest, or it may be due to intentional acts. Heroin users who lose venous access may resort to ‘skin popping’, or injecting the drug subcutaneously, which is highly damaging to tissue and frequently leads to chronic ulcers.

Periwound skin damage caused by excessive amounts of exudate and other bodily fluids can perpetuate the non-healing status of chronic wounds. Maceration, excoriation, dry (fragile) skin, hyperkeratosis, callus and eczema are frequent problems that interfere with the integrity of periwound skin. They can create a gateway for infection as well as cause wound edge deterioration preventing wound closure.

Chronic wounds may affect only the epidermis and dermis, or they may affect tissues all the way to the fascia. They may be caused by surgery or accidental trauma, or they may form as the result of systemic infection, vascular, immune, or nerve insufficiency, or comorbidities such as neoplasias or metabolic disorders. The reason a wound becomes chronic may be due in part to the body's ability to deal with the damage is overwhelmed by factors such as repeated trauma, continued pressure, ischemia, or illness.

Though much progress has been accomplished in the study of chronic wounds lately, advances in the study of their healing have lagged behind expectations. This is partly because animal studies are difficult because animals do not get chronic wounds, since they usually have loose skin that quickly contracts, and they normally do not get old enough or have contributing diseases such as neuropathy or chronic debilitating illnesses. Nonetheless, current researchers now understand some of the major factors that lead to chronic wounds, among which are ischemia, reperfusion injury, and bacterial colonization.

Factors influencing chronic wounds. Ischemia is an important factor in the formation and persistence of wounds, especially when it occurs repetitively (as it usually does) or when combined with a patient's age. Ischemia causes tissue to become inflamed and cells to release factors that attract neutrophils such as interleukins, chemokines, leukotrienes, and complement factors.

While they fight pathogens, neutrophils also release inflammatory cytokines and enzymes that damage cells. One of their important jobs is to produce Reactive Oxygen Species (ROS) to kill bacteria, for which they use an enzyme called myeloperoxidase. The enzymes and ROS produced by neutrophils and other leukocytes damage cells and prevent cell proliferation and wound closure by damaging DNA, lipids, proteins, the extracellular matrix (ECM), and cytokines that speed healing. Neutrophils remain in chronic wounds for longer than they do in acute wounds, and contribute to the fact that chronic wounds have higher levels of inflammatory cytokines and ROS. Since wound fluid from chronic wounds has an excess of proteases and ROS, the fluid itself can inhibit healing by inhibiting cell growth and breaking down growth factors and proteins in the ECM. This impaired healing response is considered uncoordinated. However, soluble mediators of the immune system (growth factors), cell-based therapies and therapeutic chemicals can propagate coordinated healing.

It has been suggested that the three fundamental factors underlying chronic wound pathogenesis are cellular and systemic changes of aging, repeated bouts of ischemia-reperfusion injury, and bacterial colonization with resulting inflammatory host response.

Since more oxygen in the wound environment allows white blood cells to produce ROS to kill bacteria, patients with inadequate tissue oxygenation, for example those who suffered hypothermia during surgery, are at higher risk for infection. The host's immune response to the presence of bacteria prolongs inflammation, delays healing, and damages tissue. Infection can lead not only to chronic wounds but also to gangrene, loss of the infected limb, and death of the patient. More recently, an interplay between bacterial colonization and increases in reactive oxygen species leading to formation and production of biofilms has been shown to the generate chronic wounds.

Like ischemia, bacterial colonization and infection damage tissue by causing a greater number of neutrophils to enter the wound site. In patients with chronic wounds, bacteria with resistances to antibiotics may have time to develop. In addition, patients that carry drug resistant bacterial strains such as methicillin-resistant Staphylococcus aureus (MRSA) have more chronic wounds.

Chronic wounds also differ in makeup from acute wounds in that their levels of proteolytic enzymes such as elastase. and matrix metalloproteinases (MMPs) are higher, while their concentrations of growth factors such as Platelet-derived growth factor and Keratinocyte Growth Factor are lower.

Since growth factors (GFs) are imperative in timely wound healing, inadequate GF levels may be an important factor in chronic wound formation. In chronic wounds, the formation and release of growth factors may be prevented, the factors may be sequestered and unable to perform their metabolic roles or degraded in excess by cellular or bacterial proteases.

Chronic wounds such as diabetic and venous ulcers may also be caused by a failure of fibroblasts to produce adequate ECM proteins and by keratinocytes to epithelialize the wound. Fibroblast gene expression is different in chronic wounds than in acute wounds.

Though most wounds require a certain level of elastase and proteases for proper healing, too high a concentration may be damaging. Leukocytes in the wound area release elastase, which increases inflammation, destroys tissue, proteoglycans, and collagen, and damages growth factors, fibronectin, and factors that inhibit proteases. The activity of elastase is increased by human serum albumin, which is the most abundant protein found in chronic wounds. However, chronic wounds with inadequate albumin may be especially unlikely to heal, so regulating the wound's levels of that protein may in the future can prove helpful in healing chronic wounds.

Excess matrix metalloproteinases, which are released by leukocytes, may also cause wounds to become chronic. MMPs break down ECM molecules, growth factors, and protease inhibitors, and thus increase degradation while reducing construction, throwing the delicate compromise between production and degradation out of balance.

Diagnosis. The vast majority of chronic wounds can be classified into three categories: venous ulcers, diabetic, and pressure ulcers. A small number of wounds that do not fall into these categories may be due to causes such as radiation poisoning or ischemia.

Venous ulcers, which usually occur in the legs, account for about 70% to 90% of chronic wounds and mostly affect the elderly. They are thought to be due to venous hypertension caused by improper function of valves that exist in the veins to prevent blood from flowing backward. Ischemia results from the dysfunction and, combined with reperfusion injury, causes the tissue damage that leads to the wounds.

Another major cause of chronic wounds, diabetes, is increasing in prevalence. Diabetics have a 15% higher risk for amputation than the general population due to, for example, chronic ulcers. Diabetes may cause neuropathy, which can inhibit nociception and the perception of pain. Thus patients may not initially notice small wounds to legs and feet, and may therefore fail to prevent infection or repeated injury. Further, diabetes causes immune compromise and damage to small blood vessels, preventing adequate oxygenation of tissue, which can cause chronic wounds. Pressure also plays a role in the formation of diabetic ulcers.

Another leading type of chronic wounds is pressure ulcers, which usually occur in people with conditions such as paralysis that inhibit movement of body parts that are commonly subjected to pressure such as the heels, shoulder blades, and sacrum. Pressure ulcers are caused by ischemia that occurs when pressure on the tissue is greater than the pressure in capillaries, and thus restricts blood flow into the area. Muscle tissue, which needs more oxygen and nutrients than skin does, shows the worst effects from prolonged pressure. As in other chronic ulcers, reperfusion injury damages tissue.

Treatment. Though treatment of the different chronic wound types varies slightly, appropriate treatment seeks to address the problems at the root of chronic wounds, including ischemia, bacterial load, and imbalance of proteases. Periwound skin issues should be assessed, and their abatement included in a proposed treatment plan. Various methods exist to ameliorate these problems, including antibiotic and antibacterial use, debridement, irrigation, vacuum-assisted closure, warming, oxygenation, moist wound healing (the term pioneered by George D. Winter), removing mechanical stress, and adding cells or other materials to secrete or enhance levels of healing factors.

It is uncertain whether intravenous metronidazole is useful in reducing foul smelling from malignant wounds. There is insufficient evidence to use silver-containing dressings or topical agents for the treatment of infected or contaminated chronic wounds.

To lower the bacterial count in wounds, therapists may use topical antibiotics, which kill bacteria and can also help by keeping the wound environment moist, which is important for speeding the healing of chronic wounds. Some researchers have experimented with the use of tea tree oil, an antibacterial agent which also has anti-inflammatory effects. Disinfectants are contraindicated because they damage tissues and delay wound contraction. Further, they are rendered ineffective by organic matter in wounds like blood and exudate and are thus not useful in open wounds.

A greater amount of exudate and necrotic tissue in a wound increases likelihood of infection by serving as a medium for bacterial growth away from the host's defenses. Since bacteria thrive on dead tissue, wounds are often surgically debrided to remove the devitalized tissue. Debridement and drainage of wound fluid are an especially important part of the treatment for diabetic ulcers, which may create the need for amputation if infection gets out of control. Mechanical removal of bacteria and devitalized tissue is also the idea behind wound irrigation, which is accomplished using pulsed lavage.

Removing necrotic or devitalized tissue is also the aim of maggot therapy, the intentional introduction by a health care practitioner of live, disinfected maggots into non-healing wounds. Maggots dissolve only necrotic, infected tissue; disinfect the wound by killing bacteria; and stimulate wound healing. Maggot therapy has been shown to accelerate debridement of necrotic wounds and reduce the bacterial load of the wound, leading to earlier healing, reduced wound odor and less pain. The combination and interactions of these actions make maggots an extremely potent tool in chronic wound care.

Negative pressure wound therapy (NPWT) is a treatment that improves ischemic tissues and removes wound fluid used by bacteria. This therapy, also known as vacuum-assisted closure, reduces swelling in tissues, which brings more blood and nutrients to the area, as does the negative pressure itself. The treatment also decompresses tissues and alters the shape of cells, causes them to express different mRNAs and to proliferate and produce ECM molecules.

Recent technological advancements produced novel approaches such as self-adaptive wound dressings that rely on properties of smart polymers sensitive to changes in humidity levels. The dressing delivers absorption or hydration as needed over each independent wound area and aids in the natural process of autolytic debridement. It effectively removes liquefied slough and necrotic tissue, disintegrated bacterial biofilm as well as harmful exudate components, known to slow the healing process. The treatment also reduces bacterial load by effective evacuation and immobilization of microorganisms from the wound bed, and subsequent chemical binding of available water that is necessary for their replication. Self-adaptive dressings protect periwound skin from extrinsic factors and infection while regulating moisture balance over vulnerable skin around the wound.

Persistent chronic pain associated with non-healing wounds is caused by tissue (nociceptive) or nerve (neuropathic) damage and is influenced by dressing changes and chronic inflammation. Chronic wounds take a long time to heal and patients can suffer from chronic wounds for many years. Chronic wound healing may be compromised by coexisting underlying conditions, such as venous valve backflow, peripheral vascular disease, uncontrolled edema and diabetes mellitus.

If wound pain is not assessed and documented, it may be ignored and/or not addressed properly. It is important to remember that increased wound pain may be an indicator of wound complications that need treatment, and therefore practitioners must constantly reassess the wound as well as the associated pain.

Optimal management of wounds requires holistic assessment. Documentation of the patient's pain experience is critical and may range from the use of a patient diary, (which should be patient driven), to recording pain entirely by the healthcare professional or caregiver. Effective communication between the patient and the healthcare team is fundamental to this holistic approach. The more frequently healthcare professionals measure pain, the greater the likelihood of introducing or changing pain management practices.

At present there are few local options for the treatment of persistent pain, whilst managing the exudate levels present in many chronic wounds. Important properties of such local options are that they provide an optimal wound healing environment, while providing a constant local low dose release of ibuprofen during wear-time.

If local treatment does not provide adequate pain reduction, it may be necessary for patients with chronic painful wounds to be prescribed additional systemic treatment for the physical component of their pain. Clinicians should consult with their prescribing colleagues referring to the WHO pain relief ladder of systemic treatment options for guidance. For every pharmacological intervention there are possible benefits and adverse events that the prescribing clinician will need to consider in conjunction with the wound care treatment team.

Blood vessels constrict in tissue that becomes cold and dilate in warm tissue, altering blood flow to the area. Thus, keeping the tissues warm is probably necessary to fight both infection and ischemia. Some healthcare professionals use ‘radiant bandages’ to keep the area warm, and care must be taken during surgery to prevent hypothermia, which increases rates of post-surgical infection.

Underlying ischemia may also be treated surgically by arterial revascularization, for example in diabetic ulcers, and patients with venous ulcers may undergo surgery to correct vein dysfunction.

Diabetics that are not candidates for surgery (and others) may also have their tissue oxygenation increased by Hyperbaric Oxygen Therapy, or HBOT, which may provide a short-term improvement in healing by improving the oxygenated blood supply to the wound. In addition to killing bacteria, higher oxygen content in tissues speeds growth factor production, fibroblast growth, and angiogenesis. However, increased oxygen levels also indicates increased production of ROS. Antioxidants, molecules that can lose an electron to free radicals without themselves becoming radicals, can lower levels of oxidants in the body and have been used with some success in wound healing.

Low level laser therapy has been repeatedly shown to significantly reduce the size and severity of diabetic ulcers as well as other pressure ulcers.

Pressure wounds are often the result of local ischemia from the increased pressure. Increased pressure also plays a role in many diabetic foot ulcerations as changes due to the disease causes the foot to suffer limited joint mobility and creates pressure points on the bottom of the foot. Effective measures to treat this includes a surgical procedure called the gastrocnemius recession in which the calf muscle is lengthened to decrease the fulcrum created by this muscle and resulting in a decrease in plantar forefoot pressure.

Since chronic wounds underexpress growth factors necessary for healing tissue, chronic wound healing may be speeded by replacing or stimulating those factors and by preventing the excessive formation of proteases like elastase that break them down.

One way to increase growth factor concentrations in wounds is to apply the growth factors directly. This generally takes many repetitions and requires large amounts of the factors, although biomaterials are being developed that control the delivery of growth factors over time. Another way is to spread onto the wound a gel of the patient's own blood platelets, which then secrete growth factors such as vascular endothelial growth factor (VEGF), insulin-like growth factor 1-2 (IGF), PDGF, transforming growth factor-β (TGF-β), and epidermal growth factor (EGF). Other treatments include implanting cultured keratinocytes into the wound to reepithelialize it and culturing and implanting fibroblasts into wounds. Some patients are treated with artificial skin substitutes that have fibroblasts and keratinocytes in a matrix of collagen to replicate skin and release growth factors.

In other cases, skin from cadavers is grafted onto wounds, providing a cover to keep out bacteria and preventing the buildup of too much granulation tissue, which can lead to excessive scarring. Though the allograft (skin transplanted from a member of the same species) is replaced by granulation tissue and is not actually incorporated into the healing wound, it encourages cellular proliferation and provides a structure for epithelial cells to crawl across. On the most difficult chronic wounds, allografts may not work, requiring skin grafts from elsewhere on the patient, which can cause pain and further stress on the patient's system.

Collagen dressings are another way to provide the matrix for cellular proliferation and migration, while also keeping the wound moist and absorbing exudate. Additionally, collagen has been shown to be chemotactic to human blood monocytes, which can enter the wound site and transform into beneficial wound-healing cells.

Since levels of protease inhibitors are lowered in chronic wounds, some researchers are seeking ways to heal tissues by replacing these inhibitors in them. Secretory leukocyte protease inhibitor (SLPI), which inhibits not only proteases but also inflammation and microorganisms like viruses, bacteria, and fungi, may prove to be an effective treatment.

Research into hormones and wound healing has shown estrogen to speed wound healing in elderly humans and in animals that have had their ovaries removed, possibly by preventing excess neutrophils from entering the wound and releasing elastase. Thus, the use of estrogen is a future possibility for treating chronic wounds.

The rate of treating of a wound may be measured or assessed according to methods known in the art. In some embodiments, the rate of wound healing is measured or assessed according to one of the methods of e.g., Cukjati et al. Medical and Biological Engineering and Computing 39 (2001): 263-271. In some embodiments, the wound healing rate is measured as the total area healed per day. In some embodiments, the wound healing device results in a wound healing rate of about 0.1 mm²/day, 0.5 mm²/day, 0.75 mm²/day, 1 mm²/day, 1.5 mm²/day, 2 mm²/day, 2.5 mm²/day, 3 mm²/day, 4 mm²/day, 5 mm²/day, 6 mm²/day, 7 mm²/day, 8 mm²/day, 9 mm²/day, 10 mm²/day, 12.5 mm²/day, 15 mm²/day, 20 mm²/day, 25 mm²/day, 30 mm²/day, 35 mm²/day, 40 mm²/day, 45 mm²/day, 50 mm²/day, 55 mm²/day, 60 mm²/day, 65 mm²/day, or 70 mm²/day in a subject, e.g., as measured by known methods in the art.

In some embodiments, the wound healing device results in an absolute wound healing rate of greater than about 0.1 mm²/day, 0.5 mm²/day, 0.75 mm²/day, 1 mm²/day, 1.5 mm²/day, 2 mm²/day, 2.5 mm²/day, 3 mm²/day, 4 mm²/day, 5 mm²/day, 6 mm²/day, 7 mm²/day, 8 mm²/day, 9 mm²/day, 10 mm²/day, 12.5 mm²/day, 15 mm²/day, 20 mm²/day, 25 mm²/day, 30 mm²/day, 35 mm²/day, 40 mm²/day, 45 mm²/day, 50 mm²/day, 55 mm²/day, 60 mm²/day, 65 mm²/day, or 70 mm²/day in a subject, e.g., as measured by known methods in the art. In some embodiments, the wound healing device results in an absolute wound healing rate of less than about 0.1 mm²/day, 0.5 mm²/day, 0.75 mm²/day, 1 mm²/day, 1.5 mm²/day, 2 mm²/day, 2.5 mm²/day, 3 mm²/day, 4 mm²/day, 5 mm²/day, 6 mm²/day, 7 mm²/day, 8 mm²/day, 9 mm²/day, 10 mm²/day, 12.5 mm²/day, 15 mm²/day, 20 mm²/day, 25 mm²/day, 30 mm²/day, 35 mm²/day, 40 mm²/day, 45 mm²/day, 50 mm²/day, 55 mm²/day, 60 mm²/day, 65 mm²/day, or 70 mm²/day in a subject, e.g., as measured by known methods in the art.

In some embodiments, the wound healing rate is measured or assessed as the percentage area of the wound healed per day. In some embodiments, the wound healing device results in a percentage healing rate of about 1% day, 2%/day, 3%/day, 4%/day, 5%/day, 6%/day, 7%/day, 8%/day, 9%/day, 10%/day, 15%/day, 20%/day, 25%/day, 30%/day, 35%/day, 40%/day, 45%/day, 50%/day, 55%/day, 60%/day, 65%/day, 70%/day, 75%/day, 80%/day, 85%/day, 90%/day, 95%/day, 99%/day in a subject, e.g., as measured by known methods in the art. In some embodiments, the wound healing device results in a percentage healing rate of greater than about 1%/day, 2%/day, 3%/day, 4%/day, 5%/day, 6%/day, 7%/day, 8%/day, 9%/day, 10%/day, 15%/day, 20%/day, 25%/day, 30%/day, 35%/day, 40%/day, 45%/day, 50%/day, 55%/day, 60%/day, 65%/day, 70%/day, 75%/day, 80%/day, 85%/day, 90%/day, 95%/day, 99%/day in a subject, e.g., as measured by known methods in the art. In some embodiments, the wound healing device results in a percentage healing rates of less than about 1%/day, 2%/day, 3%/day, 4%/day, 5%/day, 6%/day, 7%/day, 8%/day, 9%/day, 10%/day, 15%/day, 20%/day, 25%/day, 30%/day, 35%/day, 40%/day, 45%/day, 50%/day, 55%/day, 60%/day, 65%/day, 70%/day, 75%/day, 80%/day, 85%/day, 90%/day, 95%/day, 99%/day in a subject, e.g., as measured by known methods in the art.

In some embodiments, the wound healing rate is measured as the advance of the wound margin (i.e., the edge of the wound) towards the center of the wound per day. In an embodiment, the advance of the wound margin towards the center of the wound per day is about 0.1 mm/day, 0.5 mm/day, 0.75 mm/day, 1 mm/day, 1.5 mm/day, 2 mm/day, 2.5 mm/day, 3 mm/day, 4 mm/day, 5 mm/day, 6 mm/day, 7 mm/day, 8 mm/day, 9 mm/day, 10 mm/day, 12.5 mm/day, 15 mm/day, 20 mm/day, 25 mm/day, 30 mm/day, 35 mm/day, 40 mm/day, 45 mm/day, 50 mm/day, 55 mm/day, 60 mm/day, 65 mm/day, or 70 mm/day in a subject, e.g., as measured by known methods in the art. In some embodiments, the wound healing rate is measured as the advance of the wound margin (i.e., the edge of the wound) towards the center of the wound per day. In an embodiment, the advance of the wound margin towards the center of the wound per day is greater than about 0.1 mm/day, 0.5 mm/day, 0.75 mm/day, 1 mm/day, 1.5 mm/day, 2 mm/day, 2.5 mm/day, 3 mm/day, 4 mm/day, 5 mm/day, 6 mm/day, 7 mm/day, 8 mm/day, 9 mm/day, 10 mm/day, 12.5 mm/day, 15 mm/day, 20 mm/day, 25 mm/day, 30 mm/day, 35 mm/day, 40 mm/day, 45 mm/day, 50 mm/day, 55 mm/day, 60 mm/day, 65 mm/day, or 70 mm/day in a subject, e.g., as measured by known methods in the art. In some embodiments, the wound healing rate is measured as the advance of the wound margin (i.e., the edge of the wound) towards the center of the wound per day. In an embodiment, the advance of the wound margin towards the center of the wound per day is less than about 0.1 mm/day, 0.5 mm/day, 0.75 mm/day, 1 mm/day, 1.5 mm/day, 2 mm/day, 2.5 mm/day, 3 mm/day, 4 mm/day, 5 mm/day, 6 mm/day, 7 mm/day, 8 mm/day, 9 mm/day, 10 mm/day, 12.5 mm/day, 15 mm/day, 20 mm/day, 25 mm/day, 30 mm/day, 35 mm/day, 40 mm/day, 45 mm/day, 50 mm/day, 55 mm/day, 60 mm/day, 65 mm/day, or 70 mm/day in a subject, e.g., as measured by known methods in the art.

The rate of healing of a wound may be measured or assessed as the increase or decrease in the level of a biomarker e.g., a biomarker associated with the healing response, e.g., nitric oxide or TGF-β1. In an embodiment, the increase or decrease in biomarker levels may be measured or assessed by electrochemical methods (e.g., by current measurements). In an, the increase or decrease in biomarker levels may be measured or assessed by protein quantification methods known in the art, e.g., Luminex assay or Western blot.

In an embodiment, the wound healing device causes a decrease in the level of nitric oxide production by 0.1 μM/day, 0.2 μM/day, 0.5 μM/day, 0.8 μM/day, 1 μM/day, 2 μM/day, 3 μM/day, 4 μM/day, 5 μM/day, 6 μM/day, 7 μM/day, 8 μM/day, 9 μM/day, 10 μM/day, 15 μM/day, 20 25 μM/day, 30 μM/day, 40 μM/day, 50 μM/day, 60 μM/day, 70 μM/day, 80 μM/day, 90 μM/day, 100 μM/day, 150 μM/day, 200 μM/day, 250 μM/day, 300 μM/day, 350 μM/day, 400 μM/day, 450 μM/day, 500 μM/day, 550 μM/day, 600 μM/day, 650 μM/day, 700 μM/day, 750 μM/day, 800 μM/day, 850 μM/day, 900 μM/day, 950 μM/day, 1000 μM/day, 1500 μM/day, 2000 μM/day, or 2500 μM/day in a subject, e.g., as measured by known methods in the art. In an embodiment, the wound healing device causes a decrease in the level of nitric oxide production by greater than 0.1 μM/day, 0.2 μM/day, 0.5 μM/day, 0.8 μM/day, 1 μM/day, 2 μM/day, 3 μM/day, 4 μM/day, 5 μM/day, 6 μM/day, 7 μM/day, 8 μM/day, 9 μM/day, 10 μM/day, 15 μM/day, 20 25 μM/day, 30 μM/day, 40 μM/day, 50 μM/day, 60 μM/day, 70 μM/day, 80 μM/day, 90 μM/day, 100 μM/day, 150 μM/day, 200 μM/day, 250 μM/day, 300 μM/day, 350 μM/day, 400 μM/day, 450 μM/day, 500 μM/day, 550 μM/day, 600 μM/day, 650 μM/day, 700 μM/day, 750 μM/day, 800 μM/day, 850 μM/day, 900 μM/day, 950 μM/day, 1000 μM/day, 1500 μM/day, 2000 μM/day, or 2500 μM/day in a subject, e.g., as measured by known methods in the art in a subject, e.g., as measured by known methods in the art. In an embodiment, the wound healing device causes a decrease in the level of nitric oxide production by less than 0.1 μM/day, 0.2 μM/day, 0.5 μM/day, 0.8 μM/day, 1 μM/day, 2 μM/day, 3 μM/day, 4 μM/day, 5 μM/day, 6 μM/day, 7 μM/day, 8 μM/day, 9 μM/day, 10 μM/day, 15 μM/day, 20 25 μM/day, 30 μM/day, 40 μM/day, 50 μM/day, 60 μM/day, 70 μM/day, 80 μM/day, 90 μM/day, 100 μM/day, 150 μM/day, 200 μM/day, 250 μM/day, 300 μM/day, 350 μM/day, 400 μM/day, 450 μM/day, 500 μM/day, 550 μM/day, 600 μM/day, 650 μM/day, 700 μM/day, 750 μM/day, 800 μM/day, 850 μM/day, 900 μM/day, 950 μM/day, 1000 μM/day, 1500 μM/day, 2000 μM/day, or 2500 μM/day in a subject, e.g., as measured by known methods in the art.

In an embodiment, the wound healing device causes an increase in the level of nitric oxide production by 0.1 μM/day, 0.2 μM/day, 0.5 μM/day, 0.8 μM/day, 1 μM/day, 2 μM/day, 3 μM/day, 4 μM/day, 5 μM/day, 6 μM/day, 7 μM/day, 8 μM/day, 9 μM/day, 10 μM/day, 15 μM/day, 20 25 μM/day, 30 μM/day, 40 μM/day, 50 μM/day, 60 μM/day, 70 μM/day, 80 μM/day, 90 μM/day, 100 μM/day, 150 μM/day, 200 μM/day, 250 μM/day, 300 μM/day, 350 μM/day, 400 μM/day, 450 μM/day, 500 μM/day, 550 μM/day, 600 μM/day, 650 μM/day, 700 μM/day, 750 μM/day, 800 μM/day, 850 μM/day, 900 μM/day, 950 μM/day, 1000 μM/day, 1500 μM/day, 2000 μM/day, or 2500 μM/day, in a subject, e.g., as measured by known methods in the art. In an embodiment, the wound healing device causes an increase in the level of nitric oxide production by greater than 0.1 μM/day, 0.2 μM/day, 0.5 μM/day, 0.8 μM/day, 1 μM/day, 2 μM/day, 3 μM/day, 4 μM/day, 5 μM/day, 6 μM/day, 7 μM/day, 8 μM/day, 9 μM/day, 10 μM/day, 15 μM/day, 20 25 μM/day, 30 μM/day, 40 μM/day, 50 μM/day, 60 μM/day, 70 μM/day, 80 μM/day, 90 μM/day, 100 μM/day, 150 μM/day, 200 μM/day, 250 μM/day, 300 μM/day, 350 μM/day, 400 μM/day, 450 μM/day, 500 μM/day, 550 μM/day, 600 μM/day, 650 μM/day, 700 μM/day, 750 μM/day, 800 μM/day, 850 μM/day, 900 μM/day, 950 μM/day, 1000 μM/day, 1500 μM/day, 2000 μM/day, or 2500 μM/day, in a subject, e.g., as measured by known methods in the art. In an embodiment, the wound healing device causes an increase in the level of nitric oxide production by less than 0.1 μM/day, 0.2 μM/day, 0.5 μM/day, 0.8 μM/day, 1 μM/day, 2 μM/day, 3 μM/day, 4 μM/day, 5 μM/day, 6 μM/day, 7 μM/day, 8 μM/day, 9 μM/day, 10 μM/day, 15 μM/day, 20 25 μM/day, 30 μM/day, 40 μM/day, 50 μM/day, 60 μM/day, 70 μM/day, 80 μM/day, 90 μM/day, 100 μM/day, 150 μM/day, 200 μM/day, 250 μM/day, 300 μM/day, 350 μM/day, 400 μM/day, 450 μM/day, 500 μM/day, 550 μM/day, 600 μM/day, 650 μM/day, 700 μM/day, 750 μM/day, 800 μM/day, 850 μM/day, 900 μM/day, 950 μM/day, 1000 μM/day, 1500 μM/day, 2000 μM/day, or 2500 μM/day in a subject, e.g., as measured by known methods in the art.

In an embodiment, the wound healing device causes a decrease in the level of a biomarker in a subject, for example, a decrease in the level of one of interleukin 4 (IL-4), brain-derived neurotrophic factor (BDNF), tumor necrosis factor alpha (TNF-α), nerve growth factor (NGF), interleukin 12 (IL-12), interleukin 10 (IL-10), epidermal growth factor (EGF), fibroblast growth factor (FGF-2), platelet derived growth factor (PDGF), vascular endothelial growth factors (VEGF), interleukin 1 (IL-1), interleukin 6 (IL-6), connective tissue growth factor (CTGF), granulocyte-macrophage colony-stimulating factor (GM-CSF), leptin, adiponectin, interferon gamma-induced protein 10 (IP-10), nerve growth factor (NGF), insulin-like growth factor 1 (IGF-1), dopamine, acetylcholine, fractalkine, high mobility group box 1 (HMGB1), interleukin 1β (IL-1β), IL-1RA, interleukin 2 (IL-2), sIL-2Rα, interleukin 5 (IL-5), interleukin 7 (IL-7), interleukin 8 (IL-8, (CCL8), interleukin 13 (IL-13), interleukin 15 (IL-15), interleukin 17A (IL-17A), interleukin 18 (IL-18), interferon-gamma (IFN-γ), IP-10 (CXCL10), monokine induced by gamma (MIG, (CXCL9), macrophage inflammatory protein 1 alpha (MIP-1α), chemokine (C-C motif) ligand 3 (CCL3), macrophage inflammatory protein 1 beta (MIP-1β,) chemokine (C-C motif) ligands 4 (CCL4), monocyte chemoattractant protein-1 (MCP-1), chemokine (C-C motif) ligand 2 (CCL2), macrophage colony-stimulating factor (M-CSF), Eotaxin (CCL11), and active/latent transforming growth factor beta 1 (TGF-β1), by about 0.1 μM/day, 0.2 μM/day, 0.5 μM/day, 0.8 μM/day, 1 μM/day, 2 μM/day, 3 μM/day, 4 μM/day, 5 μM/day, 6 μM/day, 7 μM/day, 8 μM/day, 9 μM/day, 10 μM/day, 15 μM/day, 20 25 μM/day, 30 μM/day, 40 μM/day, 50 μM/day, 60 μM/day, 70 μM/day, 80 μM/day, 90 μM/day, 100 μM/day, 150 μM/day, 200 μM/day, 250 μM/day, 300 μM/day, 350 μM/day, 400 μM/day, 450 μM/day, 500 μM/day, 550 μM/day, 600 μM/day, 650 μM/day, 700 μM/day, 750 μM/day, 800 μM/day, 850 μM/day, 900 μM/day, 950 μM/day, 1000 μM/day, 1500 μM/day, 2000 μM/day, or 2500 μM/day, in a subject, e.g., as measured by known methods in the art.

In an embodiment, the wound healing device causes a decrease in the level of a biomarker in a subject, for example, a decrease in the level of one of interleukin 4 (IL-4), brain-derived neurotrophic factor (BDNF), tumor necrosis factor alpha (TNF-α), nerve growth factor (NGF), interleukin 12 (IL-12), interleukin 10 (IL-10), epidermal growth factor (EGF), fibroblast growth factor (FGF-2), platelet derived growth factor (PDGF), vascular endothelial growth factors (VEGF), interleukin 1 (IL-1), interleukin 6 (IL-6), connective tissue growth factor (CTGF), granulocyte-macrophage colony-stimulating factor (GM-CSF), leptin, adiponectin, interferon gamma-induced protein 10 (IP-10), nerve growth factor (NGF), insulin-like growth factor 1 (IGF-1), dopamine, acetylcholine, fractalkine, high mobility group box 1 (HMGB1), interleukin 1β (IL-1β), IL-1RA, interleukin 2 (IL-2), sIL-2Rα, interleukin 5 (IL-5), interleukin 7 (IL-7), interleukin 8 (IL-8, (CCL8), interleukin 13 (IL-13), interleukin 15 (IL-15), interleukin 17A (IL-17A), interleukin 18 (IL-18), interferon-gamma (IFN-γ), IP-10 (CXCL10), monokine induced by gamma (MIG, (CXCL9), macrophage inflammatory protein 1 alpha (MIP-1α), chemokine (C-C motif) ligand 3 (CCL3), macrophage inflammatory protein 1 beta (MIP-1β,) chemokine (C-C motif) ligands 4 (CCL4), monocyte chemoattractant protein-1 (MCP-1), chemokine (C-C motif) ligand 2 (CCL2), macrophage colony-stimulating factor (M-CSF), Eotaxin (CCL11), and active/latent transforming growth factor beta 1 (TGF-β1) by greater than about 0.1 μM/day, 0.2 μM/day, 0.5 μM/day, 0.8 μM/day, 1 μM/day, 2 μM/day, 3 μM/day, 4 μM/day, 5 μM/day, 6 μM/day, 7 μM/day, 8 μM/day, 9 μM/day, 10 μM/day, 15 μM/day, 20 25 μM/day, 30 μM/day, 40 μM/day, 50 μM/day, 60 μM/day, 70 μM/day, 80 μM/day, 90 μM/day, 100 μM/day, 150 μM/day, 200 μM/day, 250 μM/day, 300 μM/day, 350 μM/day, 400 μM/day, 450 μM/day, 500 μM/day, 550 μM/day, 600 μM/day, 650 μM/day, 700 μM/day, 750 μM/day, 800 μM/day, 850 μM/day, 900 μM/day, 950 μM/day, 1000 μM/day, 1500 μM/day, 2000 μM/day, or 2500 μM/day, in a subject, e.g., as measured by known methods in the art.

In an embodiment, the wound healing device causes a decrease in the level a biomarker in a subject, for example, a decrease in the level of one of interleukin 4 (IL-4), brain-derived neurotrophic factor (BDNF), tumor necrosis factor alpha (TNF-α), nerve growth factor (NGF), interleukin 12 (IL-12), interleukin 10 (IL-10), epidermal growth factor (EGF), fibroblast growth factor (FGF-2), platelet derived growth factor (PDGF), vascular endothelial growth factors (VEGF), interleukin 1 (IL-1), interleukin 6 (IL-6), connective tissue growth factor (CTGF), granulocyte-macrophage colony-stimulating factor (GM-CSF), leptin, adiponectin, interferon gamma-induced protein 10 (IP-10), nerve growth factor (NGF), insulin-like growth factor 1 (IGF-1), dopamine, acetylcholine, fractalkine, high mobility group box 1 (HMGB1), interleukin 1β (IL-1β), IL-1RA, interleukin 2 (IL-2), sIL-2Rα, interleukin 5 (IL-5), interleukin 7 (IL-7), interleukin 8 (IL-8, (CCL8), interleukin 13 (IL-13), interleukin 15 (IL-15), interleukin 17A (IL-17A), interleukin 18 (IL-18), interferon-gamma (IFN-γ), IP-10 (CXCL10), monokine induced by gamma (MIG, (CXCL9), macrophage inflammatory protein 1 alpha (MIP-1α), chemokine (C-C motif) ligand 3 (CCL3), macrophage inflammatory protein 1 beta (MIP-1β,) chemokine (C-C motif) ligands 4 (CCL4), monocyte chemoattractant protein-1 (MCP-1), chemokine (C-C motif) ligand 2 (CCL2), macrophage colony-stimulating factor (M-CSF), Eotaxin (CCL11), and active/latent transforming growth factor beta 1 (TGF-β1) by less than about 0.1 μM/day, 0.2 μM/day, 0.5 μM/day, 0.8 μM/day, 1 μM/day, 2 μM/day, 3 μM/day, 4 μM/day, 5 μM/day, 6 μM/day, 7 μM/day, 8 μM/day, 9 μM/day, 10 μM/day, 15 μM/day, 20 25 μM/day, 30 μM/day, 40 μM/day, 50 μM/day, 60 μM/day, 70 μM/day, 80 μM/day, 90 μM/day, 100 μM/day, 150 μM/day, 200 μM/day, 250 μM/day, 300 μM/day, 350 μM/day, 400 μM/day, 450 μM/day, 500 μM/day, 550 μM/day, 600 μM/day, 650 μM/day, 700 μM/day, 750 μM/day, 800 μM/day, 850 μM/day, 900 μM/day, 950 μM/day, 1000 μM/day, 1500 μM/day, 2000 μM/day, or 2500 μM/day in a subject, e.g., as measured by known methods in the art.

### II. DEVICES

Described herein is a wound healing device capable of inducing healing a wound in a subject. In an embodiment, the wound healing device comprises a first component comprising a cell or cells provided in an array of needle tips. The cell or cells may be engineered to secrete a wound healing factor in response to a stimulus, such as light, thereby allowing for spatial-temporal control of the delivery of the wound healing factor to a wound bed. In an embodiment, the device comprises two main components:
Engineered cells-Cells engineered to secrete wound healing factors in response to light utilizing optogenetics. Wound healing factors include all biologics important for wound healing, for example IL4, BDNF, TNF-alpha, NGF, IL12 and IL10.
Needle patch-Biomaterial needle patch into which engineered cells can be loaded; varies in dimension from a microneedle patch to a blunt needle patch; comprises at least a two-component system of first component of material for optimal cell loading (such as alginate, a hydrogel used widely in cell encapsulation) and a second component that is structurally stable (such as PDMS).
   In additional embodiments, further features may be included in or with the patch including:
Nitric oxide sensor-Micro-fabricated NO sensor array with high sensitivity and selectivity, achieving the multi-point NO mapping on the wound and directing factor release.
Electrical stimulator-High density microfabricated electrical stimulation electrodes with low impedance and high charge injection capacity to enhance muscular stem cell proliferation and differentiation.

In an embodiment, the first component may comprise a polysaccharide, e.g., an alginate, cellulose, hyaluronic acid, or chitosan. Alginate is a naturally occurring polymer comprising β-(1-4)-linked mannuronic acid and guluronic acid residues, and as a result of its high density of negatively charged carboxylates, may be cross-linked with certain cations to form a larger structure, such as a hydrogel. Alginate polymers described herein may have an average molecular weight from about 2 kDa to about 500 kDa (e.g., from about 2.5 kDa to about 175 kDa, from about 5 kDa about 150 kDa, from about 10 kDa to about 125 kDa, from about 12.5 kDa to about 100 kDa, from about 15 kDa to about 90 kDa, from about 17.5 kDa to about about 80 kDa, from about 20 kDa to about 70 kDa, from about 22.5 kDa to about 60 kDa, or from about 25 kDa to about 50 kDa). In an embodiment, the first component comprises at least 0.5%, 1%, 2%, 3%, 4%, 5%, 7.5%, 10%, 15%, 20%, 30%, 40%, 50%, 60%, 70%, 80% or more of an alginate. In an embodiment, the alginate is an ultrapure alginate (e.g., SLG20 alginate).

In an embodiment, the second component may comprise a polymer (e.g., a naturally occurring polymer or a synthetic polymer). For example, a polymer may comprise polystyrene, polyester, polycarbonate, polyethylene, polypropylene, polyfluorocarbon, nylon, polyacetylene, polyvinyl chloride (PVC), polyolefin, polyurethane, polyacrylate, polymethacrylate, polyacrylamide, polymethacrylamide, polymethyl methacrylate, poly(2-hydroxyethyl methacrylate), polysiloxane, polydimethylsiloxane (PDMS), polyhydroxyalkanoate, PEEK®, polytetrafluoroethylene, polyethylene glycol, polysulfone, polyacrylonitrile, collagen, cellulose, cellulosic polymers, polysaccharides, polyglycolic acid, poly(L-lactic acid) (PLLA), poly(lactic glycolic acid) (PLGA), polydioxanone (PDA), poly(lactic acid), hyaluronic acid, agarose, alginate, chitosan, or a blend or copolymer thereof. In an embodiment, the second component may comprise a polysaccharide (e.g., alginate, cellulose, hyaluronic acid, or chitosan). In some embodiments, the average molecular weight of the polymer is from about 2 kDa to about 500 kDa (e.g., from about 2.5 kDa to about 175 kDa, from about 5 kDa about 150 kDa, from about 10 kDa to about 125 kDa, from about 12.5 kDa to about 100 kDa, from about 15 kDa to about 90 kDa, from about 17.5 kDa to about about 80 kDa, from about 20 kDa to about 70 kDa, from about 22.5 kDa to about 60 kDa, or from about 25 kDa to about 50 kDa). The second component may comprise at least 0.5%, 1%, 2%, 3%, 4%, 5%, 7.5%, 10%, 15%, 20%, 30%, 40%, 50%, 60%, 70%, 80% or more of a polymer, e.g., a polymer described herein.

### III. OPTOGENICS AND ENGINEERED CELLS

Optogenetics is a biological technique useful for controlling the activity of neurons or other cell types with light. This control is achieved by expression of light-sensitive ion channels, pumps or enzymes specifically in the target cells. On the level of individual cells, light-activated enzymes and transcription factors may allow for precise control of biochemical signaling pathways. In systems neuroscience, the ability to control the activity of a genetically defined set of neurons has been used to understand their contribution to decision making, learning, fear memory, mating and addiction. In a first medical application of optogenetic technology, vision was partially restored in a blind patient. In a broader sense, optogenetics also includes methods to record cellular activity with genetically encoded indicators.

Optogenetics may provide millisecond-scale temporal precision which allows the experimenter to keep pace with fast biological information processing (for example, in probing the causal role of specific action potential patterns in defined neurons). Indeed, to probe the neural code, optogenetics may operate on the millisecond timescale to allow addition or deletion of precise activity patterns within specific cells in the brains of intact animals, such as mammals (e.g., a human). By comparison, the temporal precision of traditional genetic manipulations (employed to probe the causal role of specific genes within cells, via “loss-of-function” or “gain of function” changes in these genes) is rather slow, from hours or days to months. It may also be important to also have fast readouts in optogenetics that can keep pace with the optical control. This can be done with electrical recordings (“optrodes”) or with reporter proteins that are biosensors, where scientists have fused fluorescent proteins to detector proteins. Additionally, beyond its scientific impact optogenetics represents an important case study in the value of both ecological conservation (as many of the key tools of optogenetics arise from microbial organisms occupying specialized environmental niches), and in the importance of pure basic science as these opsins were studied over decades for their own sake by biophysicists and microbiologists.

The hallmark of optogenetics therefore is introduction of fast light-activated channels, pumps, and enzymes that allow temporally precise manipulation of electrical and biochemical events while maintaining cell-type resolution through the use of specific targeting mechanisms. Among the microbial opsins which can be used to investigate the function of neural systems are the channelrhodopsins (ChR2, ChR1, VChR1, and SFOs) to excite neurons and anion-conducting channelrhodopsins for light-induced inhibition. Indirectly light-controlled potassium channels have recently been engineered to prevent action potential generation in neurons during blue light illumination. Light-driven ion pumps are also used to inhibit neuronal activity, e.g., halorhodopsin (NpHR), enhanced halorhodopsins (eNpHR2.0 and eNpHR3.0), archaerhodopsin (Arch), fungal opsins (Mac) and enhanced bacteriorhodopsin (eBR).

Optogenetic control of well-defined biochemical events within behaving mammals is now also possible. Building on prior work fusing vertebrate opsins to specific G-protein coupled receptors a family of chimeric single-component optogenetic tools was created that allowed researchers to manipulate within behaving mammals the concentration of defined intracellular messengers such as cAMP and IP3 in targeted cells. Other biochemical approaches to optogenetics (crucially, with tools that displayed low activity in the dark) followed soon thereafter, when optical control over small GTPases and adenylyl cyclase was achieved in cultured cells using novel strategies from several different laboratories. Photoactivated adenylyl cyclases have been discovered in fungi and successfully used to control cAMP levels in mammalian neurons. This emerging repertoire of optogenetic actuators now allows cell-type-specific and temporally precise control of multiple axes of cellular function within intact animals.

Another important component in many optogenetic systems is hardware (e.g., integrated fiberoptic and solid-state light sources) to allow specific cell types, even deep within the brain, to be controlled in freely behaving animals. Most commonly, the latter is now often achieved using the fiberoptic-coupled diode technology introduced in 2007, though to avoid use of implanted electrodes, researchers have engineered ways to inscribe a “window” made of zirconia that has been modified to be transparent and implanted in mice skulls, to allow optical waves to penetrate more deeply to stimulate or inhibit individual neurons. To stimulate superficial brain areas such as the cerebral cortex, optical fibers or LEDs can be directly mounted to the skull of the animal. More deeply implanted optical fibers have been used to deliver light to deeper brain areas. Complementary to fiber-tethered approaches, completely wireless techniques have been developed utilizing wirelessly delivered power to headborne LEDs for unhindered study of complex behaviors in freely behaving organisms. Recent studies have examined the use of organic LEDs (OLEDs) as stimuli for optogenetics. The precise and controlled stimulation of neurons expressing microbial opsin has been demonstrated in-vitro on a time scale in the order of a millisecond. Pulsed mode operation allows neural stimulation within compatible low temperature. Moreover, organic light-emitting diodes (OLED) are suitable for implantation in the brain for their very thin thickness which can be less than 1 μm.

Optogenetics also necessarily includes the development of genetic targeting strategies such as cell-specific promoters or other customized conditionally-active viruses, to deliver the light-sensitive probes to specific populations of neurons in the brain of living animals (e.g., worms, fruit flies, mice, rats, and monkeys). In invertebrates such as worms and fruit flies, some amount of all-trans-retinal (ATR) is supplemented with food. A key advantage of microbial opsins as noted above is that they are fully functional without the addition of exogenous co-factors in vertebrates.

The technique of using optogenetics is flexible and adaptable to the experimenter's needs. For starters, experimenters genetically engineer a microbial opsin based on the gating properties (rate of excitability, refractory period, etc.) required for the experiment.

There is a challenge in introducing the microbial opsin, an optogenetic actuator, into a specific region of the organism in question. A rudimentary approach is to introduce an engineered viral vector that contains the optogenetic actuator gene attached to a recognizable promoter such as CAMKIIα. This allows for some level of specificity as cells that already contain and can translate the given promoter will be infected with the viral vector and hopefully express the optogenetic actuator gene.

Another approach is the creation of transgenic mice where the optogenetic actuator gene is introduced into mice zygotes with a given promoter, most commonly Thy1. Introduction of the optogenetic actuator at an early stage allows for a larger genetic code to be incorporated and as a result, increases the specificity of cells to be infected.

A third approach that has been developed is creating transgenic mice with Cre recombinase, an enzyme that catalyzes recombination between two lox-P sites. Then by introducing an engineered viral vector containing the optogenetic actuator gene in between two lox-P sites, only the cells containing the Cre recombinase will express the microbial opsin. This last technique has allowed for multiple modified optogenetic actuators to be used without the need to create a whole line of transgenic animals every time a new microbial opsin is needed.

After the introduction and expression of the microbial opsin, depending on the type of analysis being performed, application of light can be placed at the terminal ends or the main region where the infected cells are situated. Light stimulation can be performed with a vast array of instruments from light-emitting diodes (LEDs) or diode-pumped solid-state laser (DPSS). These light sources are most commonly connected to a computer through a fiber optic cable. Recent advances include the advent of wireless head-mounted devices that also apply LED to targeted areas and as a result give the animal more freedom of mobility to reproduce in vivo results.

Moreover, fiber-based approaches can now offer simultaneous single-cellular resolution optical stimulation and Calcium imaging. This enables researchers to visualize and manipulate the activity of single neurons while preserving naturalistic animal behaviours. Further, these techniques allow one to record in multiple deep brain regions at the same using GRIN lenses connected via optical fiber to an externally positioned photodetector and photostimulator.

Moreover, mathematical modelling shows that selective expression of opsin in specific cell types can dramatically alter the dynamical behavior of the neural circuitry. In particular, optogenetic stimulation that preferentially targets inhibitory cells can transform the excitability of the neural tissue from Type 1—where neurons operate as integrators—to Type 2 where neurons operate as resonators.

Type 1 excitable media sustain propagating waves of activity whereas Type 2 excitable media do not. The transformation from one to the other explains how constant optical stimulation of primate motor cortex elicits gamma-band (40-80 Hz) oscillations in the manner of a Type 2 excitable medium. Yet those same oscillations propagate far into the surrounding tissue in the manner of a Type 1 excitable medium.

It may be difficult to target opsin to defined subcellular compartments, e.g., the plasma membrane, synaptic vesicles, or mitochondria. Restricting the opsin to specific regions of the plasma membrane such as dendrites, somata or axon terminals would provide a more robust understanding of neuronal circuitry.

Analogously to how natural light-gated ion channels such as channelrhodopsin-2 allows optical control of ion flux, which is especially useful in neuroscience, natural light-controlled signal transduction proteins also allow optical control of biochemical pathways, including both second-messenger generation and protein-protein interactions, which is especially useful in studying cell and developmental biology. In 2002, the first example of using photoproteins from another organism for controlling a biochemical pathway was demonstrated using the light-induced interaction between plant phytochrome and phytochrome-interacting factor (PIF) to control gene transcription in yeast. By fusing phytochrome to a DNA-binding domain and PIF to a transcriptional activation domain, transcriptional activation of genes recognized by the DNA-binding domain could be induced by light. This study anticipated aspects of the later development of optogenetics in the brain, for example, by suggesting that “Directed light delivery by fiber optics has the potential to target selected cells or tissues, even within larger, more-opaque organisms.” The literature has been inconsistent as to whether control of cellular biochemistry with photoproteins should be subsumed within the definition of optogenetics, as optogenetics in common usage refers specifically to the control of neuronal firing with opsins, and as control of neuronal firing with opsins postdates and utilizes distinct mechanisms from control of cellular biochemistry with photoproteins.

In addition to phytochromes, which are found in plants and cyanobacteria, LOV domains (Light-oxygen-voltage-sensing domain) from plants and yeast and cryptochrome domains from plants are other natural photosensory domains that have been used for optical control of biochemical pathways in cells. In addition, a synthetic photosensory domain has been engineered from the fluorescent protein Dronpa for optical control of biochemical pathways. In photosensory domains, light absorption is either coupled to a change in protein-protein interactions (in the case of phytochromes, some LOV domains, cryptochromes, and Dronpa mutants) or a conformational change that exposes a linked protein segment or alters the activity of a linked protein domain (in the case of phytochromes and some LOV domains). Light-regulated protein-protein interactions can then be used to recruit proteins to DNA, for example to induce gene transcription or DNA modifications, or to the plasma membrane, for example to activate resident signaling proteins. CRY2 also clusters when active, so has been fused with signaling domains and subsequently photoactivated to allow for clustering-based activation. The LOV2 domain of Avena sativa (common oat) has been used to expose short peptides or an active protein domain in a light-dependent manner. Introduction of this LOV domain into another protein can regulate function through light induced peptide disorder. The asLOV2 protein, which optogenetically exposes a peptide, has also been used as a scaffold for several synthetic light induced dimerization and light induced dissociation systems (iLID and LOVTRAP, respectively).¹ The systems can be used to control proteins through a protein splitting strategy. Photodissociable Dronpa domains have also been used to cage a protein active site in the dark, uncage it after cyan light illumination, and recage it after violet light illumination.

The ability to optically control signals for various time durations is being explored to elucidate how cell signaling pathways convert signal duration and response to different outputs. Natural signaling cascades are capable of responding with different outputs to differences in stimulus timing duration and dynamics.^{[147]} For example, treating PC12 cells with epidermal growth factor (EGF, inducing a transient profile of ERK activity) leads to cellular proliferation whereas introduction of nerve growth factor (NGF, inducing a sustained profile of ERK activity) leads to differentiation into neuron-like cells.^{[148]} This behavior was initially characterized using EGF and NGF application, but the finding has been partially replicated with optical inputs. In addition, a rapid negative feedback loop in the RAF-MEK-ERK pathway was discovered using pulsatile activation of a photoswitchable RAF engineered with photodissociable Dronpa domains.

Red and NIR light-responsive). Phytochromes are photoreceptors that utilize tetrapyrrole chromophores such as biliverdin IXα (BV) or phycocyanobilin (PCB) to absorb red and NIR light and induce reversible conformational changes in the protein structure. One common photoreceptor used to control gene expression is Phytochrome protein B (PhyB), and its interaction partner Phytochrome-Interacting Factor 3 (PIF3) that dimerise under red light and dissociate under far-red light. By fusing DNA-Binding Domains (DBDs) and transActivation Domains (ADs) to distinct PhyB and PIF modules, red light inducible two-hybrid systems have been used to activate gene expression in yeast and mammalian cells.

In bacteria, Two-Component signaling Systems (TCSs) comprising of a natural or engineered light-responsive kinase and a downstream response regulator are used. For example, Cph8 is a light-responsive kinase engineered by replacing the osmosensory domain of a membrane-bound histidine kinase with the Cph1 phytochrome. In the absence of light, Cph8 phosphorylates the transcription factor OmpR and promotes gene expression, while in the presence of red light the kinase activity and gene expression are inactivated. The natural cyanobacteriochrome TCS and CcaS/CcaR regulate gene expression according to green and red light in a similar fashion.

Most red light-responsive gene expression systems require a PCB chromophore to function. PCB can be added to the growth media and taken up by cells or, alternatively, gene cassettes encoding enzymes for PCB biosynthesis are used to enable cells to synthesize PCB from intracellular heme. NIR-responsive phytochromes utilize BV chromophores that are produced endogenously by mammalian cells. Such NIR-responsive gene expression systems can be derived from BphP1 and PpsR2 proteins, the latter being sequestered by BphP1 in NIR light and released in the presence of red light or absence of light. NIR-responsive transcription factors have been created by fusing BphP1 and PpsR2 to DBDs and Ads. These constructs activate reporter gene expression in bacteria, mammalian cells, and mice.

LOV domain proteins and cryptochromes are two distinct protein families that differ in their protein architecture, yet both use blue light absorbing flavin chromophores to induce conformational changes in the protein. Cryptochrome-based systems are based primarily on the interaction of Cryptochrome protein 2 (CRY2) with Cryptochrome-Interacting Basic-helix-loop-helix protein (CIB1).

Blue light responsive elements. Blue light-responsive transcription systems are also known, such as TCS implementing an engineered light-sensitive kinase called YF1. This construct was created by replacing an oxygen-sensing domain of a natural kinase with the YvtA LOV domain. In the absence of light, YF1 phosphorylated and activated a transcription factor, whereas in the presence of blue-light, kinase activity and gene expression were inactivated. Using this system as a model, a bacterial repression switch, pDusk, and activation switch, pDawn, were developed. Blue light-regulated gene expression in eukaryotic cells, however, is typically controlled with photoactivatable transcription factors. Flavin-binding, Kelch domain, F-box protein (FKF1) and its interaction partner GIGANTEA (GI) were can be fused to ADs and DBDs and for blue light-activated transcription control in mammalian cells. Similar light-activated effector proteins based on CRY2/CIB1, and smaller LOV domain proteins also function in this way. Another is Vivid (VVD), a photoreceptor that rapidly and reversibly forms homodimers. These constructs have been fused to a variety of DBDs and effector domains to control both transcription and translation in mammalian cells, bacteria, and yeast.

Light-inducible transcription factors often depend on the heterodimerization of two different proteins to modulate gene expression. Single component blue light-activated transcription factors do exist, however, and are typically derived from AsLOV2 or EL222. Upon exposure to blue light, both AsLOV2 and EL222 undergo conformational changes involving the release of an alpha helix from the LOV domain. Nuclear localization signals (NLS) or nuclear export signals (NES) inserted into the Ja helix of AsLOV2 permit switching of cellular locations using blue light. Genes can then be expressed via the import of AsLOV2-based transactivators into the nucleus, or export of AsLOV2-based repressors into the cytoplasm. Alternatively, uncaging of the 4a helix in EL222 is accompanied by the release of a DBD and exposure of a dimerization interface. EL222 fused to an AD has been shown to rapidly induce gene expression in mammalian cells and zebrafish embryos following irradiation and has also been used in yeast to improve their chemical production capabilities. Single component gene expression systems in bacteria and cell-free expression systems have used the transcription factor activity of wild type EL222.

Technologies that target specific sites on the genome may allow for precise control of endogenous genes, for example the Clustered, Regularly Interspaced, Short Palindromic Repeat (CRISPR)-associated nuclease Cas9 system, ZF proteins, Transcription Activator-Like Effectors (TALEs), and recombinases. Photocages and naturally light-sensitive proteins have been incorporated into these technologies for spatiotemporal control of gene knockouts and transcription.

CRISPR-Cas systems can be modified to create light-activated CRISPR-Cas9 systems with both Ca9 and gRNA modifications. For example, a protector DNA, containing 2-nitrobenzyls in the backbone and bound to gRNA has permitted controlled gene knockout in mammalian cells. Attaching 2-nitrobenzyls to the Watson-Crick face of nucleobases in the gRNA provided improved gene editing in zebrafish cells. To control the Cas9 nuclease, a 2-nitrobenzyl-modified amino acid was installed using an orthogonal tRNA/tRNA synthetase pair. Cas9 has also been covalently linked to UCNPs, via a 2-nitrobenzyl photocage, allowing NIR-activated gene editing and reduction of tumor size in mice.

Naturally light-sensitive proteins have been used with both Cas9 and dCas9 to achieve light-activated gene knockout and transcriptional control, respectively. These rely on the dimerization of split Cas9 domains and/or dCas9 with ADs via photoreceptors, such as CRY2/CIB1 and magnet proteins. Other light-activated CRISPR-Cas9 systems employ light-activated phosphorylation or cyclic diguanylate monophosphate (c-di-GMP) signalling cascades, as well as a dimeric green fluorescent protein, pdDronpa.

ZF proteins recognize a specific 3-base pair DNA sequence; similarly, individual TALE proteins each recognize a single base pair. Thus, effector proteins can be targeted to specific locations on a genome by fusing them to ZF and TALE domains. Gene expression in mammalian cells has been controlled through fusion of ZFs to GI and an AD to FKF1. TALEs and ADs using the CRY2/CIB1 pair have been used to regulate gene expression in mouse and rat cells. In addition, incorporating artificial recognition, such as LoxP sites, in a host cell genom, light activated recombinases can control the expression of targeted genes. Some systems use 2-nitrobenzyl-photocaged tamoxifen that can control light-dependent recombination and gene expression in mouse cells. CRY2/CIB1, magnet, or VVD pairs have also been fused to split Cre and Flp recombinase domains to control gene expression in mouse and zebrafish cells.

Disclosed herein are wound healing devices comprising engineered cells, e.g., a living cell, e.g., an active cell, and methods of making or manufacturing such medical patches comprising engineered cells.

Engineered cells are described herein and have advantageous properties that can be exploited for use in the present disclosure. In an embodiment, the engineered cells maintain a density or number of cells that does not vary by more than about 10, 20, 30, 40 or 50% over a preselected period of time, in in vitro culture, or applied to a subject, e.g., to a wound bed, e.g., over about 0.1, 0.5, 1, 2, 3, 4, 5, 10, 20, 30, 45, 60, or 90 days or more.

In some embodiments, the engineered cells produce an agent, such as a protein or small molecule. Exemplary proteins or small molecules include interleukin 4 (IL-4), brain-derived neurotrophic Factor (BDNF), tumor necrosis factor alpha (TNF-α), nerve growth factor (NGF), interleukin 12 (IL-12), interleukin 10 (IL-10), epidermal growth factor (EGF), fibroblast growth factor (FGF-2), Platelet derived growth factor (PDGF), vascular endothelial growth factors (VEGF), interleukin 1 (IL-1), interleukin 6 (IL-6), connective tissue growth factor (CTGF), Granulocyte-macrophage colony-stimulating factor (GM-CSF), leptin, adiponectin, interferon gamma-induced protein 10 (IP-10), Nerve growth factor (NGF), insulin-like growth factor 1 (IGF-1), dopamine, acetylcholine, fractalkine, high mobility group box 1 (HMGB1), interleukin 1β (IL-1β), IL-1RA, interleukin 2 (IL-2), sIL-2Rα, interleukin 5 (IL-5), interleukin 7 (IL-7), interleukin 8 (IL-8, CCL8), interleukin 13 (IL-13), interleukin 15 (IL-15), interleukin 17A (IL-17A), interleukin 18 (IL-18), interferon-gamma (IFN-γ), monokine induced by gamma (MIG,CXCL9), macrophage inflammatory protein 1 alpha (MIP-1^{α}), chemokine (C-C motif) ligand 3CCL3), macrophage inflammatory protein 1 beta (MIP-1β,) chemokine (C-C motif) ligands 4CCL4), monocyte chemoattractant protein-1 (MCP-1), chemokine (C-C motif) ligand 2 (CCL2), macrophage colony-stimulating factor (M-CSF), eotaxin (CCL11), active/latent transforming growth factor beta 1 (TGF-β1), and/or lactic acid other metabolites for glycolysis.

In an embodiment, the engineered cell is an autologous, allogeneic, or xenogeneic cell. In an embodiment, the engineered cell is an immortalized cell or is derived from an immortalized cell. In an embodiment, the engineered cell is a non-immortalized cell or is derived from a non-immortalized cell. In an embodiment, the engineered cell is cell derived from a less differentiated cell, e.g., a pluripotent cell, multipotent cell, a stem cell, an embryonic stem cell, a mesenchymal stem cell, an induced pluripotent stem cell; a reprogrammed cell, a reprogrammed stem cell, or a cell derived from reprogrammed stem cells. In an embodiment, the engineered cell is derived from a naturally a derived source, xenotissue, allotissue, a cadaver, a cell line, or a primary cell.

The engineered cell may express an agent, such as a protein or nucleic acid, or produce a metabolic product. An engineered cell can be a mammalian cell, e.g., a human cell.

In an embodiment, the engineered cell is a mammalian cell that comprises at least one exogenous transcription unit, which may be present in an extra-chromosomal expression vector, or integrated into one or more chromosomal sites in the cell. In an embodiment, the transcription unit comprises a promoter operably linked to a coding sequence for a polypeptide. In an embodiment, the polypeptide coding sequence is a naturally-occurring sequence (e.g., wild-type of native) or a codon-optimized sequence. In an embodiment, the transcription unit is present in an extra-chromosomal expression vector. In an embodiment, the engineered cell comprises two, three, four or more copies of the exogenous transcription unit that are integrated in tandem in the same site of the cell genome.

In an embodiment, the exogenous transcription unit comprises a coding sequence for a light-responsive protein, e.g., a protein whose activity depends on induction by various wavelengths of light, e.g., a protein that exhibits a light-induced structural change e.g., a protein that exhibits light-induced allostery. In an embodiment, the exogenous transcription unit comprises a coding sequence for a polypeptide that is responsive to blue light. In an embodiment, the exogenous transcription unit comprises a coding sequence for a transcription factor that is responsive to blue light. In an embodiment, the coding sequence of the polypeptide is EL222.

In an embodiment, the exogenous transcription unit comprises a coding sequence for a polypeptide that is responsive to red light. In an embodiment, the exogenous transcription unit a coding sequence for a transcription factor that is responsive to red light. In an embodiment, the polypeptide is a cytokine.

In an embodiment, the engineered cells described herein comprise 1, 2, 3, 4, 5 or more exogenous transcription units, which may be present in an extra-chromosomal expression vector, or integrated into one or more chromosomal sites in the cell. In an embodiment, the engineered cells comprise one or more exogenous transcription units, which may be present in an extra-chromosomal expression vector, or integrated into one or more chromosomal sites in the cell. In an embodiment, the engineered cells comprise two exogenous transcription units, which may be present in an extra-chromosomal expression vector, or integrated into one or more chromosomal sites in the cell. In an embodiment, the transcription units comprise a promoter operably linked to a coding sequence for a polypeptide. In an embodiment, each of the exogenous transcription units comprises a coding sequence for a light-responsive protein. In an embodiment, the engineered cells comprise the same exogenous transcription unit. In an embodiment, the engineered cells comprise different exogenous transcription units.

In an embodiment, the first exogenous transcription unit comprises a coding sequence for a polypeptide that is responsive to blue light and the second exogenous transcription unit comprises a coding sequence for a polypeptide that is responsive to red light. In an embodiment, the first exogenous transcription unit comprises a coding sequence for a transcription factor that is responsive to blue light and the second exogenous transcription unit comprises a coding sequence for a transcription factor that is responsive to red light.

In an embodiment, the exogenous transcription unit comprises a coding sequence for a phytochrome. In an embodiment, the exogenous transcription unit comprises a coding sequence for a native or “wild-type” phytochrome. In an embodiment, the exogenous transcription unit comprises a coding sequence for a mutated phytochrome, i.e., a coding sequence having at least 95% identity to a native or “wild-type” phytochrome. In an embodiment, the exogenous transcription unit transcription unit comprises a promoter operably linked to coding sequence for a phytochrome described in e.g., Quail, Peter H. “Phytochrome photosensory signalling networks.” Nature reviews Molecular cell biology 3.2 (2002): 85-93 or U.S. Pat. No. 9,296,797, each of which is incorporated herein by reference in its entirety.

In an embodiment, the exogenous transcription unit comprises a coding sequence for a phototropin, e.g., a flavoprotein e.g., a protein comprising a flavin chromophore, e.g., a polypeptide comprising a light-oxygen-voltage-sensing domain. In an embodiment, the exogenous transcription unit comprises a coding sequence for a native or “wild-type” phototropin. In an embodiment, the exogenous transcription unit comprises a coding sequence for a mutated phototropin, i.e., a coding sequence having at least 95% identity to a native or “wild-type” phototropin.

In an embodiment, the exogenous transcription unit transcription unit comprises a promoter operably linked to coding sequence for one of eBDNF, TNF-α, NGF, and IL4. In an embodiment, cells comprising the PhB/PIF6 red-light optogenetic system have a secretion rate of about 0.05 pg eBDNF/cell/hour, 0.01 pg eBDNF/cell/hour, 0.02 pg eBDNF/cell/hour, 0.03 pg eBDNF/cell/hour, 0.04 pg eBDNF/cell/hour, 0.05 pg eBDNF/cell/hour, 0.06 pg eBDNF/cell/hour, 0.07 pg eBDNF/cell/hour, 0.08 pg eBDNF/cell/hour, 0.09 pg eBDNF/cell/hour, 0.1 pg eBDNF/cell/hour, 0.2 pg eBDNF/cell/hour, 0.3 pg eBDNF/cell/hour, 0.4 pg eBDNF/cell/hour, 0.5 pg eBDNF/cell/hour, 0.6 pg eBDNF/cell/hour, 0.7 pg eBDNF/cell/hour, 0.8 pg eBDNF/cell/hour, 0.9 pg eBDNF/cell/hour, or 1 pg eBDNF/cell/hour.

In an embodiment, cells comprising the PhB/PIF6 red-light optogenetic system have a secretion rate of greater than about 0.05 pg eBDNF/cell/hour, 0.01 pg eBDNF/cell/hour, 0.02 pg eBDNF/cell/hour, 0.03 pg eBDNF/cell/hour, 0.04 pg eBDNF/cell/hour, 0.05 pg eBDNF/cell/hour, 0.06 pg eBDNF/cell/hour, 0.07 pg eBDNF/cell/hour, 0.08 pg eBDNF/cell/hour, 0.09 pg eBDNF/cell/hour, 0.1 pg eBDNF/cell/hour, 0.2 pg eBDNF/cell/hour, 0.3 pg eBDNF/cell/hour, 0.4 pg eBDNF/cell/hour, 0.5 pg eBDNF/cell/hour, 0.6 pg eBDNF/cell/hour, 0.7 pg eBDNF/cell/hour, 0.8 pg eBDNF/cell/hour, 0.9 pg eBDNF/cell/hour, or 1 pg eBDNF/cell/hour.

In an embodiment, cells comprising the PhB/PIF6 red-light optogenetic system have a secretion rate of less than about 0.05 pg eBDNF/cell/hour, 0.01 pg eBDNF/cell/hour, 0.02 pg eBDNF/cell/hour, 0.03 pg eBDNF/cell/hour, 0.04 pg eBDNF/cell/hour, 0.05 pg eBDNF/cell/hour, 0.06 pg eBDNF/cell/hour, 0.07 pg eBDNF/cell/hour, 0.08 pg eBDNF/cell/hour, 0.09 pg eBDNF/cell/hour, 0.1 pg eBDNF/cell/hour, 0.2 pg eBDNF/cell/hour, 0.3 pg eBDNF/cell/hour, 0.4 pg eBDNF/cell/hour, 0.5 pg eBDNF/cell/hour, 0.6 pg eBDNF/cell/hour, 0.7 pg eBDNF/cell/hour, 0.8 pg eBDNF/cell/hour, 0.9 pg eBDNF/cell/hour, or 1 pg eBDNF/cell/hour.

In an embodiment, cells comprising the EL222 blue-light optogenetic system have a secretion rate of about 0.05 pg eBDNF/cell/hour, 0.01 pg eBDNF/cell/hour, 0.02 pg eBDNF/cell/hour, 0.03 pg eBDNF/cell/hour, 0.04 pg eBDNF/cell/hour, 0.05 pg eBDNF/cell/hour, 0.06 pg eBDNF/cell/hour, 0.07 pg eBDNF/cell/hour, 0.08 pg eBDNF/cell/hour, 0.09 pg eBDNF/cell/hour, 0.1 pg eBDNF/cell/hour, 0.2 pg eBDNF/cell/hour, 0.3 pg eBDNF/cell/hour, 0.4 pg eBDNF/cell/hour, 0.5 pg eBDNF/cell/hour, 0.6 pg eBDNF/cell/hour, 0.7 pg eBDNF/cell/hour, 0.8 pg eBDNF/cell/hour, 0.9 pg eBDNF/cell/hour, or 1 pg eBDNF/cell/hour.

In an embodiment, cells comprising the EL222 blue-light optogenetic system have a secretion rate of greater than about 0.05 pg eBDNF/cell/hour, 0.01 pg eBDNF/cell/hour, 0.02 pg eBDNF/cell/hour, 0.03 pg eBDNF/cell/hour, 0.04 pg eBDNF/cell/hour, 0.05 pg eBDNF/cell/hour, 0.06 pg eBDNF/cell/hour, 0.07 pg eBDNF/cell/hour, 0.08 pg eBDNF/cell/hour, 0.09 pg eBDNF/cell/hour, 0.1 pg eBDNF/cell/hour, 0.2 pg eBDNF/cell/hour, 0.3 pg eBDNF/cell/hour, 0.4 pg eBDNF/cell/hour, 0.5 pg eBDNF/cell/hour, 0.6 pg eBDNF/cell/hour, 0.7 pg eBDNF/cell/hour, 0.8 pg eBDNF/cell/hour, 0.9 pg eBDNF/cell/hour, or 1 pg eBDNF/cell/hour.

In an embodiment, cells comprising the EL222 blue-light optogenetic system have a secretion rate of less than about 0.05 pg eBDNF/cell/hour, 0.01 pg eBDNF/cell/hour, 0.02 pg eBDNF/cell/hour, 0.03 pg eBDNF/cell/hour, 0.04 pg eBDNF/cell/hour, 0.05 pg eBDNF/cell/hour, 0.06 pg eBDNF/cell/hour, 0.07 pg eBDNF/cell/hour, 0.08 pg eBDNF/cell/hour, 0.09 pg eBDNF/cell/hour, 0.1 pg eBDNF/cell/hour, 0.2 pg eBDNF/cell/hour, 0.3 pg eBDNF/cell/hour, 0.4 pg eBDNF/cell/hour, 0.5 pg eBDNF/cell/hour, 0.6 pg eBDNF/cell/hour, 0.7 pg eBDNF/cell/hour, 0.8 pg eBDNF/cell/hour, 0.9 pg eBDNF/cell/hour, or 1 pg eBDNF/cell/hour.

In an embodiment, an engineered cell is derived from a culture in which at least 10, 20, 30, 40, 50, 60, 79, 80, 90, 95, 98, or 99% of the cells in the culture are engineered cells, e.g., RPE cells, e.g., engineered RPE cells. In an embodiment, a culture comprises engineered cells, e.g., RPE cells, or engineered RPE cells, and a second cell type, e.g., a feeder cell or a contaminating cell. In an embodiment, an engineered cell is an RPE cell, e.g., an engineered RPE cell derived from an individual, e.g., the same or a different individual to whom the cells are administered.

An active cell can be derived from any of a variety of strains. Exemplary strains of RPE cells include ARPE-19 cells, ARPE-19-SEAP-2-neo cells, RPE-J cells, and hTERT RPE-1 cells. In some embodiments, the engineered cell is an ARPE-19 cell or derived from an ARPE-19 cell. In some embodiments, the engineered cell is an engineered ARPE-19 cell, which is derived from the ARPE-19 (ATCC® CRL-2302™) cell line.

### IV. NEEDLE PATCH ARRAY

Microneedles or Microneedle Patches or Microarray Patches are micron-scaled medical devices used to administer vaccines, drugs and other therapeutic agents. While microneedles were initially explored for transdermal drug delivery applications their use has been extended for the intraocular, vaginal, transungual, cardiac, vascular, gastrointestinal and intracochlear delivery of drugs. Microneedles are constructed through various methods usually involving photolithographic or processes micromolding. These methods involve etching microscopic structure into resin or silicon in order to cast microneedles. Microneedles are made from a variety of material ranging from silicon, titanium, stainless steel, and polymers. Some microneedles are made of a drug to be delivered to the body but are shaped into a needle so they will penetrate the skin. The microneedles range in size, shape, and function but are all used as an alternative to other delivery methods like the conventional hypodermic needle or other injection apparatus.

Microneedles are usually applied through even single needle or small arrays. The arrays used are a collection of microneedles, ranging from only a few microneedles to several hundred, attached to an applicator, sometimes a patch or other solid stamping device. The arrays are applied to the skin of patients and are given time to allow for the effective administration of drugs. Microneedles are an easier method for physicians as they require less training to apply and because they are not as hazardous as other needles, making the administration of drugs to patients safer and less painful while also avoiding some of the drawbacks of using other forms of drug delivery, such as risk of infection, production of hazardous waste, or cost.

Microneedles were first mentioned in a 1998 paper demonstrating that microneedles could penetrate the uppermost layer (stratum corneum) of the human skin and were therefore suitable for the transdermal delivery of therapeutic agents. Subsequent research into microneedle drug delivery has explored the medical and cosmetic applications of this technology through its design. This early paper sought to explore the possibility of using microneedles in the future for vaccination. Since then researchers have studied microneedle delivery of insulin, vaccines, anti-inflammatoires, and other pharmaceuticals. In dermatology, microneedles are used for scarring treatment with skin rollers.

The major goal of any microneedle design is to penetrate the skin's outermost layer, the stratum corneum (10-15 μm). Microneedles are long enough to cross the stratum corneum but not so long that they stimulate nerves which are located deeper in the tissues and therefore cause no or little pain.

Research has shown that there is a limit on the type of drugs that can be delivered through intact skin. Only compounds with a relatively low molecular weight, like the common allergen nickel (130 Da), can penetrate the skin. Compounds that weigh more than 500 Da cannot penetrate the skin.

Since their conceptualization in 1998, several advances have been made in terms of the variety of types of microneedles that can be fabricated. The 4 main types of microneedles are Solid, Hollow, Coated and Dissolvable/Dissolving.

Solid. This type of array is designed as a two part system; the microneedle array is first applied to the skin to create microscopic wells just deep enough to penetrate the outermost layer of skin, and then the drug is applied via transdermal patch. Solid microneedles are already used by dermatologists in collagen induction therapy, a method which uses repeated puncturing of the skin with microneedles to induce the expression and deposition of the proteins, collagen and elastin in the skin.

Hollow. Hollow microneedles are similar to solid microneedles in material. They contain reservoirs that deliver the drug directly into the site. Since the delivery of the drug is dependent on the flow rate of the microneedle, there is a possibility that this type of array could become clogged by excessive swelling or flawed design. This design also increases the likelihood of buckling under the pressure of, and therefore failing to deliver any drugs.

Coated. Just like solid microneedles, coated microneedles are usually designed from polymers or metals. In this method the drug is applied directly to the microneedle array instead of being applied through other patches or applicators. Coated microneedles are often covered in other surfactants or thickening agents to assure that the drug is delivered properly. Some of the chemicals used on coated microneedles are known irritants. While there is risk of local inflammation to the area where the array was, the array can be removed immediately with no harm to the patient.

Dissolvable. In a more recent adaptation of the microneedle design, dissolvable microneedles encapsulate the drug in a nontoxic polymer which dissolves once inside the skin. This polymer would allow the drug to be delivered into the skin and could be broken down once inside the body. Pharmaceutical companies and researchers have begun to study and implement polymers such as Fibroin, a silk-based protein that can be molded into structures like microneedles and dissolved once in the body.

Microneedle patches or arrays may be fabricated according to methods known in the art, e.g., micro molding, photolithography, 3D printing (e.g., additive manufacturing), drawing lithography, solvent casting, mold-based etching, and lithography. Combinations of the above methods may be used in the fabrication of a microneedle patch. In an embodiment, microneedles and microneedle arrays are produced by three-dimensional (3D) printing using a suitable resin. In some embodiments, microneedles and microneedle arrays are produced by three-dimensional (3D) printing using a biocompatible resin.

The wound healing device described herein may comprise a microneedle patch of any size. In some embodiments, the microneedle array is about 0.5 cm, 1 cm, 1.5 cm, 2 cm, 2.5 cm, 3 cm, 3.5 cm, 4 cm, 4.5 cm, 5 cm, 5.5 cm, 6 cm, 6.5 cm, 7 cm, 7.5 cm, 8 cm, 8.5 cm, 9 cm, 9.5 cm, 10 cm, 10.5 cm, 11 cm, 11.5 cm, 12 cm, 12.5 cm, 13 cm, 13.5 cm, 14 cm, e.g., in its longest linear dimension, e.g., its length or width. In some embodiments, the microneedle array is less than about 0.5 cm, less than 1 cm, less than 1.5 cm, less than 2 cm, less than 2.5 cm, less than 3 cm, less than 3.5 cm, less than 4 cm, less than 4.5 cm, less than 5 cm, less than 5.5 cm, less than 6 cm, less than 6.5 cm, less than 7 cm, less than 7.5 cm, less than 8 cm, less than 8.5 cm, less than 9 cm, less than 9.5 cm, less than 10 cm, less than 10.5 cm, less than 11 cm, less than 11.5 cm, less than 12 cm, less than 12.5 cm, less than 13 cm, less than 13.5 cm, less than 14 cm, e.g., in its longest linear dimension, e.g., its length or width. In some embodiments, the microneedle array is greater than about 0.5 cm, greater than 1 cm, greater than 1.5 cm, greater than 2 cm, greater than 2.5 cm, greater than 3 cm, greater than 3.5 cm, greater than 4 cm, greater than 4.5 cm, greater than 5 cm, greater than 5.5 cm, greater than 6 cm, greater than 6.5 cm, greater than 7 cm, greater than 7.5 cm, greater than 8 cm, greater than 8.5 cm, greater than 9 cm, greater than 9.5 cm, greater than 10 cm, greater than 10.5 cm, greater than 11 cm, greater than 11.5 cm, greater than 12 cm, greater than 12.5 cm, greater than 13 cm, greater than 13.5 cm, greater than 14 cm, e.g., in its longest linear dimension, e.g., its length or width.

The wound healing device described herein may comprise a microneedle patch of any size. In some embodiments, the microneedle array is about 0.25 cm², 0.5 cm², 1 cm², 1.5 cm², 2 cm², 2.5 cm², 3 cm², 3.5 cm², 4 cm², 4.5 cm², 5 cm², 5.5 cm², 6 cm², 6.5 cm², 7 cm², 7.5 cm², 8 cm², 8.5 cm², 9 cm², 9.5 cm², 10 cm², 10.5 cm² 11 cm, 11.5 cm², 12 cm², 12.5 cm², 13 cm², 13.5 cm², 14 cm², 15 cm², 16 cm², 17 cm², 18 cm², 19 cm², 20 cm², 25 cm², 30 cm², 35 cm², or 40 cm², e.g., in surface area. In some embodiments, the microneedle array is greater than about 0.25 cm², 0.5 cm², 1 cm², 1.5 cm², 2 cm², 2.5 cm², 3 cm², 3.5 cm², 4 cm², 4.5 cm², 5 cm², 5.5 cm², 6 cm², 6.5 cm², 7 cm², 7.5 cm², 8 cm², 8.5 cm², 9 cm², 9.5 cm², 10 cm², 10.5 cm² 11 cm, 11.5 cm², 12 cm², 12.5 cm², 13 cm², 13.5 cm², 14 cm², 15 cm², 16 cm², 17 cm², 18 cm², 19 cm², 20 cm², 25 cm², 30 cm², 35 cm², or 40 cm², e.g., in surface area. In some embodiments, the microneedle array is less than about 0.25 cm², 0.5 cm², 1 cm², 1.5 cm², 2 cm², 2.5 cm², 3 cm², 3.5 cm², 4 cm², 4.5 cm², 5 cm², 5.5 cm², 6 cm², 6.5 cm², 7 cm², 7.5 cm², 8 cm², 8.5 cm², 9 cm², 9.5 cm², 10 cm², 10.5 cm² 11 cm, 11.5 cm², 12 cm², 12.5 cm², 13 cm², 13.5 cm², 14 cm², 15 cm², 16 cm², 17 cm², 18 cm², 19 cm², 20 cm², 25 cm², 30 cm², 35 cm², or 40 cm², e.g., in surface area.

The wound healing device may comprise a microneedle array comprising individual needles. In some embodiments, the microneedle array comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more needles. In some embodiments, the microneedle array comprises greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 needles. In some embodiments, the microneedle array comprises less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 needles.

The microneedle patch may comprise needles of different shapes. Each needle may be in any shape as long as it can pierce the skin or reach the wound bed. Each microneedle can be various shapes such as a cone, pyramid, cylinder, prism, or pencil-like shape (meaning a shape having a column body and a cone-shaped end portion). In some embodiments, the microneedle has a conical or pyramidal shape.

In some embodiments, a single microneedle is provided on the support base. In other cases, a plurality of microneedles may be disposed closely together on the support base. When a plurality of microneedles are disposed, the microneedles may be arranged in an array. As used herein, the term “array” means that microneedles are arranged in a specific pattern, for example in a matrix arrangement, concentric circle arrangement, or random arrangement.

As described herein, microneedles can be hollow or solid. Microneedles can be produced by any method that yields microneedles and microneedle arrays that are smooth and sharp enough to penetrate wounded skin. The resin can be a thermoplastic resin. In some embodiments, the resin is biodegradable.

The microneedle array described herein may comprise needles with different lengths, e.g., as measured from a base or support. In some embodiments, the needles are about 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 900 μm, 950 μm, 1000 μm, 1250 μm, 1500 μm, 1750 μm, 2000 μm in, e.g., in length, e.g., as measured from a base or support. In some embodiments, the needles are greater than about 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 900 μm, 950 μm, 1000 μm, 1250 μm, 1500 μm, 1750 μm, 2000 μm in, e.g., in length, e.g., as measured from a base or support. In some embodiments, the needles are less than about 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 900 μm, 950 μm, 1000 μm, 1250 μm, 1500 μm, 1750 μm, 2000 μm in, e.g., in length, e.g., as measured from a base or support.

The microneedle array described herein may comprise needle tips with different radii. In some embodiments, the needle tip radii are about 10 μm, 25 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 250 μm, 300 μm, 350 μm, 400 μm, 450 μm, 500 μm, 550 μm, 600 μm, 650 μm, 700 μm, 750 μm, 800 μm, 900 μm, 950 μm, 1000 μm in, e.g., diameter or longest linear dimension. In some embodiments, the needle tip radii are greater than about 10 μm, 25 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 250 μm, 300 μm, 350 μm, 400 μm, 450 μm, 500 μm, 550 μm, 600 μm, 650 μm, 700 μm, 750 μm, 800 μm, 900 μm, 950 μm, 1000 μm in, e.g., diameter or longest linear dimension. In some embodiments, the needle tip radii are less than about 10 μm, 25 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 250 μm, 300 μm, 350 μm, 400 μm, 450 μm, 500 μm, 550 μm, 600 μm, 650 μm, 700 μm, 750 μm, 800 μm, 900 μm, 950 μm, 1000 μm in, e.g., diameter or longest linear dimension.

The wound healing patches described herein may comprise reservoirs suitable for disposing additional materials e.g., cells, e.g., engineered cells, e.g., polymer solutions comprising engineered cells. In some embodiments, the wound healing patch may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more reservoirs for, e.g., disposing additional materials, e.g., cells. In some embodiments, the wound healing patch may comprise greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 reservoirs for, e.g., disposing additional materials, e.g., cells. In some embodiments, the wound healing patch may comprise less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 reservoirs for, e.g., disposing additional materials, e.g., cells.

The wound healing patches described herein may comprise reservoirs suitable for disposing additional materials e.g., cells, e.g., engineered cells, e.g., polymer solutions comprising engineered cells. In some embodiments, the wound healing patch may comprise reservoirs capable of containing a volume of a substance, e.g., a polymer, e.g., a hydrogel, e.g., a hydrogel comprising cells, e.g., a liquid. In some embodiments, the reservoirs may comprise μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, 550 μL, 600 μL, 650 μL, 700 μL, 750 μL, 800 μL, 850 μL, 900 μL, 950 μL, 1000 μL, 1100 μL, 1200 μL, 1300 μL, 1400 μL, 1500 μL, 1600 μL, 1700 μL, 1800 μL, 1900 μL, 2000 μL, 2100 μL, 2200 μL, 2300 μL, 2400 μL, 2500 μL, 2600 μL, 2700 μL, 2800 μL, 2900 μL, 3000 μL, 3100 μL, 3200 μL, 3300 μL, 3400 μL, 3500 μL, 3600 μL, 3700 μL, 3800 μL, 3900 μL, or 4000 μL.

In some embodiments, the wound healing patch may comprise reservoirs capable of containing a volume of a substance, e.g., a polymer, e.g., a hydrogel, e.g., a hydrogel comprising cells, e.g., a liquid. In some embodiments, the reservoirs may comprise greater than 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, 550 μL, 600 μL, 650 μL, 700 μL, 750 μL, 800 μL, 850 μL, 900 μL, 950 μL, 1000 μL, 1100 μL, 1200 μL, 1300 μL, 1400 μL, 1500 μL, 1600 μL, 1700 μL, 1800 μL, 1900 μL, 2000 μL, 2100 μL, 2200 μL, 2300 μL, 2400 μL, 2500 μL, 2600 μL, 2700 μL, 2800 μL, 2900 μL, 3000 μL, 3100 μL, 3200 μL, 3300 μL, 3400 μL, 3500 μL, 3600 μL, 3700 μL, 3800 μL, 3900 μL, or 4000 μL.

In some embodiments, the wound healing patch may comprise reservoirs capable of containing a volume of a substance, e.g., a polymer, e.g., a hydrogel, e.g., a hydrogel comprising cells, e.g., a liquid. In some embodiments, the reservoirs may comprise less than 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, 550 μL, 600 μL, 650 μL, 700 μL, 750 μL, 800 μL, 850 μL, 900 μL, 950 μL, 1000 μL, 1100 μL, 1200 μL, 1300 μL, 1400 μL, 1500 μL, 1600 μL, 1700 μL, 1800 μL, 1900 μL, 2000 μL, 2100 μL, 2200 μL, 2300 μL, 2400 μL, 2500 μL, 2600 μL, 2700 μL, 2800 μL, 2900 μL, 3000 μL, 3100 μL, 3200 μL, 3300 μL, 3400 μL, 3500 μL, 3600 μL, 3700 μL, 3800 μL, 3900 μL, or 4000 μL.

### V. BIOSENSORS

A biosensor is an analytical device, used for the detection of chemical or biological substances, that typically combines a biological component with a physicochemical detector. The analyte can be any biological or chemical element, e.g., often an enzyme, hormone, growth factor, antibody, nucleic acid, etc., for which the sensor provides a detection agent. The detector element can then transforms the detected presence of an analyte into a signal, often working in a physicochemical way, such as in optical, piezoelectric, electrochemical, or electro-chemiluminescence modes, etc., such that measurement as well as quantification of the analyte can be achieved. The biosensor device can connect with associated electronics or signal processors that are primarily responsible for the display of the results in a user-friendly way. These are usually custom-designed and manufactured to suit the different working principles of biosensors. A well-known examples is glucose monitoring sensors for diabetic patients.

### A. Nitric Oxide Sensors

Nitric oxide (nitrogen monoxide) is a signaling molecule involved a number of physiological and pathological processes, including those in the in the nervous, immune, and cardiovascular systems. It is a powerful vasodilator with a half-life of a few seconds in the blood. Standard pharmaceuticals such as nitroglycerine and amyl nitrite are precursors to nitric oxide. Low levels of nitric oxide production are typically due to ischemic damage in the liver. Platelet-derived factors, shear stress, acetylcholine, and cytokines stimulate the production of NO by endothelial nitric oxide synthase (eNOS). NO is also a neurotransmitter and has been associated with neuronal activity, partially mediates macrophage cytotoxicity against microbes and tumor cells, and is implicated in pathophysiologic states as diverse as septic shock, hypertension, stroke, and neurodegenerative diseases.

One very important and helpful function for NO is in wound healing. Wound healing is a complex process that involves coordinated interactions between diverse immunological and biological systems and long-term/chronic wound healing is challenging clinical problem affecting millions of people per year and leading to significant health costs. In recent years, nitric oxide (NO) has emerged as a critical molecule in wound healing, with NO levels increasing rapidly after skin damage and gradually decreasing as the healing process progresses. Studies have shown that the NO significantly accelerated wound healing by promoting angiogenesis and collagen deposition in wounded tissue.

A chemical sensor array is a sensor architecture with multiple sensor components that create a pattern for analyte detection from the additive responses of individual sensor components. There exist several types of chemical sensor arrays including electronic, optical, acoustic wave, and potentiometric devices, which are described below. These chemical sensor arrays can employ multiple sensor types that are cross-reactive or tuned to sense specific analytes.

The signal(s) coming from an array sensor must be processed and compared with already-known patterns. Many techniques are useful in processing array data including principal component analysis (PCA), least square analysis, and more recently training of neural networks and utilization of machine learning for pattern development and identification. Machine learning has been a more recent development for generation and recognition of patterns for chemical sensor array data. The method of data analysis chosen depends on a variety of factors including sensing parameters, desired use of the information (quantitative or qualitative), and the method of detection which can be classified under four major types of chemical sensor array: electronic, optical, acoustic wave, and electrochemical sensor arrays.

One type of chemical sensor array relies on modulation of an electronic signal for signal acquisition. This type of chemical sensor array often utilizes a semiconductive material such as metal-oxide semiconductors, conductive polymers, nanomaterials, or framework materials such as metal-organic and covalent-organic frameworks. One of the simplest device architectures for an electronic chemical sensor is a chemiresistor, and other architectures include capacitors and transistors; these materials have a resistance which can be altered through physisorption or chemisorption of target molecules and thus a measurable signal as a change in electrical current, capacitance, or voltage. The inventors will employ NO sensor in the wound device to both monitor wound healing and to guide further treatment, such as electrical stimulation and deliver of growth factors, cytokines, chemokines and lymphokines through light-controlled expression systems.

The wound healing device described herein may detect nitric oxide concentration at one or more positions in the wound bed. In some embodiments, the wound healing device is capable of detecting nitric oxide concentration at different placement positions on the wound.

In an embodiment, the wound healing device is capable of measuring NO at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, or 64 different positions on the wound, e.g., measure current from this number of electrodes. In an embodiment, the wound healing device is capable of measuring NO at less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, or 64 different positions on the wound, e.g., measure current from this number of electrodes. In an embodiment, the wound healing device is capable of measuring NO at greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, or 64 different positions on the wound, e.g., measure current from this number of electrodes.

In some embodiments, the nitric oxide sensor comprises a 3-dimentional fuzzy graphene (3DFG), gold, or platinum electrode. In an embodiment, the nitric oxide sensor comprises a 3DGF electrode.

In an embodiment, the NO sensor detects NO through an electrochemical reaction. In an embodiment of the invention, the NO detector detects NO through an oxidation reaction.

The nitric oxide sensing electrodes may be coated with semi-permeable membranes to improve selectivity of nitric oxide signal. Appropriate semi-permeable membranes are known in the art. Exemplary semi-permeable membranes of the current invention include Nafion, Eugenol, FePc and 5AN1.

The nitric oxide sensors may additionally comprise a catalyst for the oxidation of NO. In some embodiments, the catalyst is a metalloporphyrin. In a preferred embodiment of the invention, the catalyst is Ni-porphyrin (e.g., Nickel (II) tetrakis(3-methoxy-4-hydroxyphenyl) porphyrin, Ni-TMHPP).

Nitric oxide sensors can measure nitric oxide concentration over time via chronoamperometry measurements. In some embodiments, the nitric oxide sensors monitor NO production for 0.1 days, 0.2 days, 0.3 days, 0.4 days, 0.5 days, 0.6 days, 0.7 days, 0.8 days, 0.9 days, 1 days, 1.1 days, 1.2 days, 1.3 days, 1.4 days, 1.5 days, 1.6 days, 1.7 days, 1.8 days, 1.9 days, 2.0 days, 2.1 days, 2.2 days, 2.3 days, 2.4 days, 2.5 days, 2.6 days, 2.7 days, 2.8 days, 2.9 days, 3.0 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days. In some embodiments, the nitric oxide sensors monitor NO production for more than 0.1 days, 0.2 days, 0.3 days, 0.4 days, 0.5 days, 0.6 days, 0.7 days, 0.8 days, 0.9 days, 1 days, 1.1 days, 1.2 days, 1.3 days, 1.4 days, 1.5 days, 1.6 days, 1.7 days, 1.8 days, 1.9 days, 2.0 days, 2.1 days, 2.2 days, 2.3 days, 2.4 days, 2.5 days, 2.6 days, 2.7 days, 2.8 days, 2.9 days, 3.0 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days. In some embodiments, the nitric oxide sensors monitor NO production for less than 0.1 days, 0.2 days, 0.3 days, 0.4 days, 0.5 days, 0.6 days, 0.7 days, 0.8 days, 0.9 days, 1 days, 1.1 days, 1.2 days, 1.3 days, 1.4 days, 1.5 days, 1.6 days, 1.7 days, 1.8 days, 1.9 days, 2.0 days, 2.1 days, 2.2 days, 2.3 days, 2.4 days, 2.5 days, 2.6 days, 2.7 days, 2.8 days, 2.9 days, 3.0 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days.

### B. TGF-β Sensors

TGF-β is another important molecule in wound healing. Matharu et al., Anal. Chem. 86 (17): 8865-8874 (2014) reported the development of a cell-culture/biosensor platform consisting of aptamer-modified Au electrodes integrated with reconfigurable microfluidics for monitoring of transforming growth factor-beta 1 (TGF-β1), an important inflammatory and pro-fibrotic cytokine. Aptamers were thiolated, labeled with redox reporters, and self-assembled on gold surfaces. The biosensor was determined to be specific for TGF-β1 with an experimental detection limit of 1 ng/mL and linear range extending to 250 ng/mL. The sensor was miniaturized and integrated with human hepatic stellate cells inside microfluidic devices. This microsystem with integrated aptasensors and used to monitor TGF-β1 release from activated stellate cells over the course of 20 hours.

### VI. ELECTRICAL STIMULATION AS WOUND THERAPY

Electrotherapy is any use of electrical energy as a medical treatment. In medicine, the term electrotherapy can apply to a variety of treatments, including the use of electrical devices such as deep brain stimulators for neurological disease. The term has also been applied specifically to the use of electric current to speed wound healing. Additionally, the term “electrotherapy” or “electromagnetic therapy” has also been applied to a range of alternative medical devices and treatments.

In recent years, electrical stimulation (ES) for the treatment of both acute and chronic wounds has gained prominence in the literature. This is because an injury to the skin results in a flow of current through the wound pathway that generates a lateral electrical field. This has been called the “current of injury” or “skin battery” effect which is thought to be significant in initiating repair. Numerous studies now suggest that ES therapy in conjunction with standard wound care can provide improved clinical outcomes. In this context, ES is defined as the application of electrical current through electrodes placed on the skin either near or directly on the wound. Beneficial effects on different phases of cutaneous wound healing in both chronic and acute contexts have been reported. ES appears to reduce infection, improve cellular immunity, increase perfusion, and accelerate cutaneous wound healing. Important parameters for using of ES devices in wound healing are voltage, current, mode and length of time of application. Also, mono- or bipolar and bi- or tri-electrodes have been used with different types of wounds being receptive to different modalities.

While a number of ES devices and methods of application exist, the majority of studies apply the electrodes directly on the skin, often directly onto the wound. Different modalities and electrical waveforms include direct current (DC), alternating current (AC), high-voltage pulsed current (HVPC), and low-intensity direct current (LIDC). One of the common is transcutaneous electrical nerve stimulation (TENS), previously used extensively for treating pain. Frequency rhythmic electrical modulation systems (FREMS) is another form of transcutaneous electrotherapy that varies the pulse, frequency, duration, and voltage. Recently, an electrobiofeedback device has been used in the treatment of acute cutaneous wound healing and for reducing the symptoms associated with abnormal skin scarring. This “Fenzian system” device is characterized as waveform found to appear as degenerate waves (DW), which degenerate over time.

The wound healing patch described herein may provide electrical stimulation to biological structures. In some embodiments, the biological structure is a wound bed, subdermal muscle, or nerve bundles. In an embodiment, the biological structure is a wound bed. In an embodiment, the biological structure is subdermal muscle. In an embodiment, the biological structure is a nerve bundle (e.g., sciatic nerve). In an embodiment, electrical stimulation of the biological structure (e.g., wound bed) is facilitated by high density electrode arrays.

The electrode arrays described herein may be arranged in any configuration. In an embodiment, the electrodes are arranged in a symmetrical arrangement or an asymmetrical arrangement. In an embodiment, the electrodes are arranged at random. In an embodiment, the high-density electrode arrays have a density of about 0.1 mm⁻², 0.2 mm⁻², 0.3 mm⁻², 0.4 mm⁻², 0.5 mm⁻², 0.6 mm⁻², 0.7 mm⁻², 0.8 mm⁻², 0.9 mm⁻², 1 mm⁻², 2 mm⁻², 3 mm-2, or 4 mm-2. In an embodiment, the high-density electrode arrays have a density of greater than about 0.1 mm⁻², 0.2 mm⁻², 0.3 mm⁻², 0.4 mm⁻², 0.5 mm⁻², 0.6 mm⁻², 0.7 mm⁻², 0.8 mm⁻², 0.9 mm⁻², 1 mm², 2 mm⁻², 3 mm-2, or 4 mm-2. In an embodiment, the high-density electrode arrays have a density of less than about 0.1 mm⁻², 0.2 mm⁻², 0.3 mm⁻², 0.4 mm⁻², 0.5 mm⁻², 0.6 mm⁻², 0.7 mm⁻², 0.8 mm⁻², 0.9 mm², 1 mm², 2 mm 2, 3 mm-2, or 4 mm-2 electrodes.

The high-density electrode arrays described herein may comprise electrodes of different shapes and sizes. In an embodiment, the high-density electrode array comprises circular electrodes or elliptical electrodes. In an embodiment, the high-density electrode arrays comprise an electrode that is about 10 μm, 20 μm, 50 μm, 75 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1000 μm, 1250 μm, 1500 μm, 1750 μm, 2000 μm, 2500 μm, or 3000 μm, e.g., in diameter or longest linear dimension. In an embodiment, the high-density electrode arrays comprise an electrode that is greater than about 10 μm, 20 μm, 50 μm, 75 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1000 μm, 1250 μm, 1500 μm, 1750 μm, 2000 μm, 2500 μm, or 3000 μm, e.g., in diameter or longest linear dimension. In an embodiment, the high-density electrode arrays comprise an electrode that is less than about 10 μm, 20 μm, 50 μm, 75 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1000 μm, 1250 μm, 1500 μm, 1750 μm, 2000 μm, 2500 μm, or 3000 μm, e.g., in diameter or longest linear dimension.

The high-density electrode arrays described herein may comprise any material suitable for conducting electric charge. In an embodiment, the electrodes comprise a metal, metal oxide, or group 14 element. In an embodiment, the electrodes comprise a metal or metal oxide. In an embodiment, the electrodes of the high-density electrode arrays comprise a metal selected from the group consisting of gold or platinum. In an embodiment, the electrodes of the high-density electrode arrays comprise a metal oxide selected from the group consisting of indium tin oxide or titanium nitride. In an embodiment, the electrodes comprise a group 14 element. In some embodiments, the electrodes comprise a carbon allotrope (e.g., graphene) or silicon. In an embodiment, the electrodes comprise three-dimensional fuzzy graphene (3DFG)_

The high-density electrode arrays described herein may comprise electrodes that are coated with one or more layers of an organic polymer. In an embodiment, the electrodes are coated with one layer of an organic polymer. In an embodiment of the invention, the electrodes comprise gold coated by one or more layers of organic polymer. In an embodiment of the invention, the electrodes comprise 3DFG coated by one or more layers of organic polymer.

In an embodiment, the electrodes comprise gold coated by poly(3,4-ethylenedioxythiophene) polystyrene sulfonate (PEDOT:PSS). In another embodiment, the electrodes comprise gold coated by PEDOT:PSS and an additional organic polymer. In yet another embodiment of the invention, the electrodes comprise gold coated by a first layer of PEDOT:PSS and a second layer of Xerogel.

The high-density electrode arrays described herein may be characterized by methods known in the art. In some embodiments, the electrodes are characterized by impedance measurements. Electrochemical impedance provides a direct estimate of the recording capabilities of an electrode. Designing bioelectronics interfaces with low impedances is important for enhancing their signal-to-noise ratio. In some embodiments, the electrodes are characterized by impedance measurements over the frequency range of 1 to 10⁵ Hz. In some embodiments, the electrodes are characterized by impedance measurements at frequencies of 1 kHz or less. In some embodiments, impedance values of the electrodes at 1 kHz are from about 5×10³ to 2×10⁵ Ohm.

The capacitive and faradaic currents generated at the cell membrane during the cathodal current phase at the stimulating electrode lead to the depolarization of the membrane and result in electrical stimulation. To prevent damage of biological entities during electrical stimulation, the maximum cathodic potential drop (E_{mc}) and the maximum anodic potential drop (Eₘₐ) across the electrode-electrolyte interface should not exceed the electrolysis window for H₂O under any stimulating current pulse. The magnitude of the stimulation current pulses that can be safely applied at the electrode-electrolyte interface is governed by the charge injection capacity (CIC) of the microelectrodes. Therefore, CIC is determined as the amount of charge that can be injected to the electrode without crossing E_{mc} and is assessed through voltage transient measurements. The charge injection capacity of the electrodes described herein ay be measured according to methods known in the art.

### VII. PATIENT SELECTION

Described herein are wound healing devices useful for decreasing wound healing time in a subject. In an embodiment, the subject may have a disease, disorder, or condition resulting in a wound. For example, the subject may have or be identified as having an immune disorder, a proliferative disorder, endocrine disorder, neurological disorder, cardiovascular disorder, pulmonary disorder, or dermatological disorder. For example, the subject may have diabetes, a cancer, eczema, psoriasis. In an embodiment, the disease, disorder, or condition is an immune disorder, e.g., rheumatoid arthritis, lupus, multiple sclerosis, psoriasis, Graves' disease, scleroderma, Crohn's disease, or celiac disease. In an embodiment, the disease, disorder, or condition is a proliferative disorder, e.g., cancer, e.g., basal cell carcinoma or squamous cell carcinoma. In an embodiment, the disease, disorder, or condition is melanoma. In an embodiment, the disease, disorder, or condition is an endocrine disorder, e.g., diabetes, Cushing's disease, hypothyroidism, or hyperthyroidism. In an embodiment, the disease, disorder, or condition is a dermatological disorder, e.g., atopic dermatitis, actinic keratosis, rosacea, eczema, cellulitis, or impetigo.

In an embodiment, the subject has a detectable amount of a biomarker, e.g., a cytokine, e.g., nitric oxide. In an embodiment, the biomarker is selected from nitric oxide, IL-4, BDNF, TNF-α, NGF, IL-12, IL-10, EGF, FGF-2, PDGF, VEGF, IL-1, IL-6, CTGF, GM-CSF, leptin, adiponectin, IP-10, NGF, IGF-1, dopamine, acetylcholine, fractalkine, HMGB1, IL-1β, IL-1RA, IL-2, sIL-2Rα, IL-5, IL-7, IL-8 (CCL8), IL-13, IL-15, IL-17A, IL-18, IFN-γ, IP-10 (CXCL10), MIG (CXCL9), MIP-1α (CCL3), MIP-1β (CCL4), MCP-1 (CCL2), M-CSF, Eotaxin (CCL11), active/latent TGF-β1, and/or lactic acid. In an embodiment, the subject shows a reduction/increase in the level of a biomarker.

In an embodiment, upon administration of the wound healing device, the subject exhibits a detectable reduction in the size of the wound, e.g., compared to a reference standard, e.g., compared to the size of the wound prior to administration of the wound healing device.

In an embodiment, upon administration of the wound healing device, the wound exhibits a change in a wound feature e.g., compared to a reference standard, e.g., compared to the wound feature prior to administration of the wound healing device. Exemplary wound features include size of the wound, depth of the wound, texture of the wound, level of hardening, scarring, scabbing of the wound, decrease in infection of the wound, color of wound, increase in tissue growth (e.g., muscle or skin). In an embodiment, upon administration of the wound healing device, the subject exhibits a decrease in wound healing time. In an embodiment, the subject has one wound or a plurality of wounds. In an embodiment, the subject receives one course of treatment of a wound healing device described herein.

### VIII. COMBINATION THERAPY

In the context of the present disclosure, it also is contemplated that the wound healing patch described herein could be used similarly in conjunction with other standard wound treatments. It also may prove effective, in particular, to combine the wound healing patch with other therapies, such as those mentioned in Section I above.

To effect an improved or enhanced therapy using the wound healing patch of the present invention and another agent to therapy, one would generally contact a wound with the wound healing patch at the same time or with one modality preceding the other such that, if applied separately, one would generally ensure that a significant period of time did not expire between the time of each treatment such that the wound healing patch and other would still be able to exert an advantageously combined effect on the cell. In such instances, it is contemplated that one would contact the wound with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the wound healing patch or the other agent will be desired. Various combinations may be employed, where the wound healing patch according to the present disclosure is “A” and the other therapy is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated.

The wound healing device described herein may be combined with any clinically acceptable wound treatment article (e.g., negative pressure therapy devices, bandages, films, adhesives, and the like). In some embodiments, the wound healing device and the other clinically acceptable superficial wound treatment article are used concurrently. In some embodiments, the wound healing device is used before the clinically acceptable superficial wound treatment article. In some embodiments, the wound healing device is used after the clinically acceptable superficial wound treatment article. In some embodiments, the clinically acceptable superficial wound treatment article is a bandage. In some embodiments, the clinically acceptable superficial wound treatment article is a thin film. In some embodiments, the clinically acceptable superficial wound treatment article is a Tegaderm™, a transparent film dressing.

The wound healing device described herein may be combined with any clinically acceptable wound treatment modality (e.g., hyperbaric oxygen treatment, low energy laser, ultrasound/ultraviolet treatment, skin grafts, drug administration, hormone administration and the like). In some embodiments, the wound healing device and the other clinically acceptable wound treatment modality are used concurrently. In some embodiments, the wound healing device is used before the clinically acceptable wound treatment modality. In some embodiments, the wound healing device is used after the clinically acceptable wound treatment modality.

### IX. METHODS OF MAKING

The present disclosure further comprises methods for making a wound healing patch described herein, e.g., a wound healing patch comprising a first component comprising a cell, a second component, and, optionally, one or more sensors or actuators (e.g., a nitric oxide sensor, electrodes for delivering electrical stimulation, and the like). In some embodiments, the method of making the wound healing patch comprises (i). fabricating a first component (e.g., through 3D printing, additive manufacturing and the like); (ii). contacting a polymer solution (e.g., a hydrogel-forming polymer) comprising a cell or a plurality of cells; and (iii) optionally laminating one or more electrode arrays onto the first component.

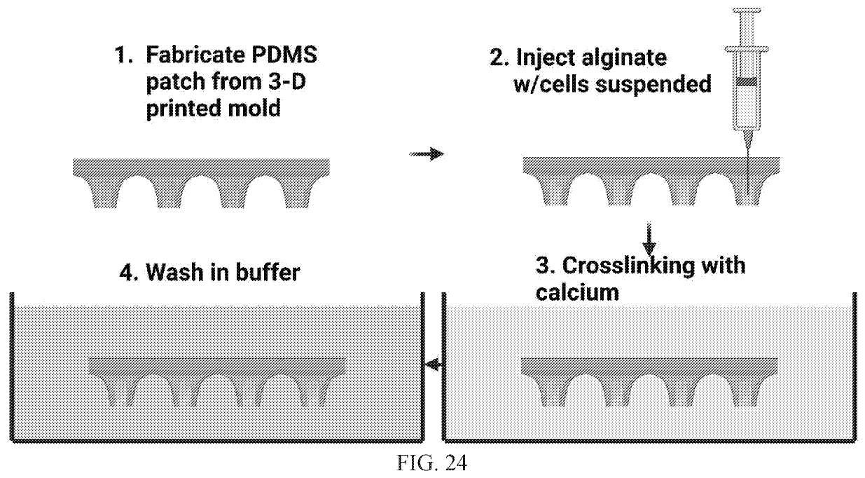

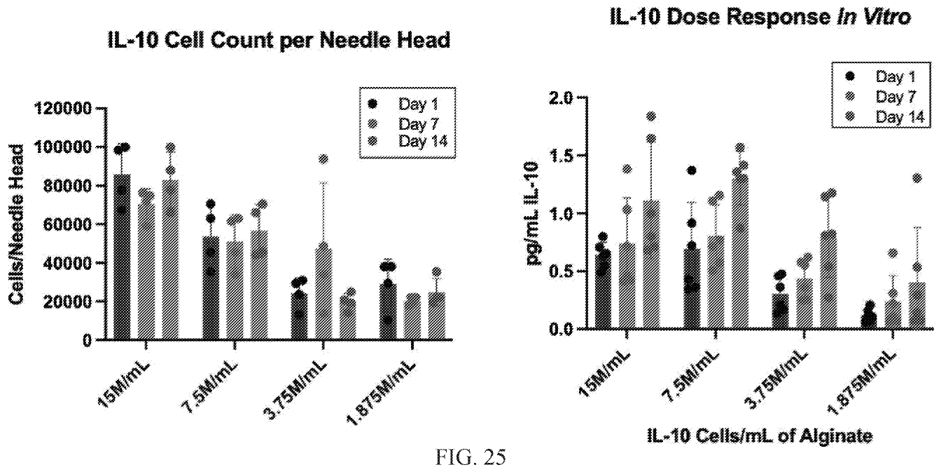

Briefly, a Form2 three-dimensional (3-D) printer was used to print the top and bottom molds of the patch. Black V4 resin was employed for molds with a layer thickness setting of 25 microns. Rafts and supports were generated automatically. Immediately after the print finished and before removal from the baseplate, the molds were submerged into an isopropanol bath for 30 minutes. Molds were gently removed from the baseplate with a metal spatula and were submerged again in an isopropanol bath and vigorously swirled for 1 minute. Molds were subsequently brushed and cleansed with warm water to remove excess resin. The steps comprising submersion of the mold in the isopropanol bath and the brushing and cleansing steps with warm water were repeated twice for a cumulative total of three washes with isopropanol and water. Excess water was removed via flow of compressed nitrogen gas. Molds were placed in an oven at 60° C. overnight. The molds were optionally clamped to a plane (level) surface to mitigate mold deformation during the drying process when forming large molds (cf. FIG. 24).

PDMS silicone elastomer, e.g., the commercially available grade SYLGARD 184 from Dow® Chemical, was prepared by mixing the base and polymerizing agent in a 8:1 ratio. PDMS and polymerizing agent were stirred until the solution was an opaque, pearly white. The PDMS was then degassed in a vacuum chamber, and the PDMS solution was poured into the molds, such that that the level of the solution was congruent with the top of the mold. Excess bubbles were removed by allowing the PDMS to sit in the mold before curing. If air bubbles persisted a needle syringe was optionally employed for removal. PDMS was then allowed to cure at 65° C. overnight.

Thereafter, the cured PDMS was demolded and cell-laden alginate was incorporated and subsequently crosslinked in the patch. Cell laden-alginate comprising 15 million cells/ml in 1.4% SLG20 alginate, wherein the cells are ARPE-19 cells engineered to secrete the indicated factor, was injected into the hollow chamber of the needle tips or pillars, and then submerged in crosslinking solution (CaCl₂)) for 10 minutes. Patches were then washed in phosphate-buffered saline (PBS) thrice and placed in media for storage in a cell culture incubator.

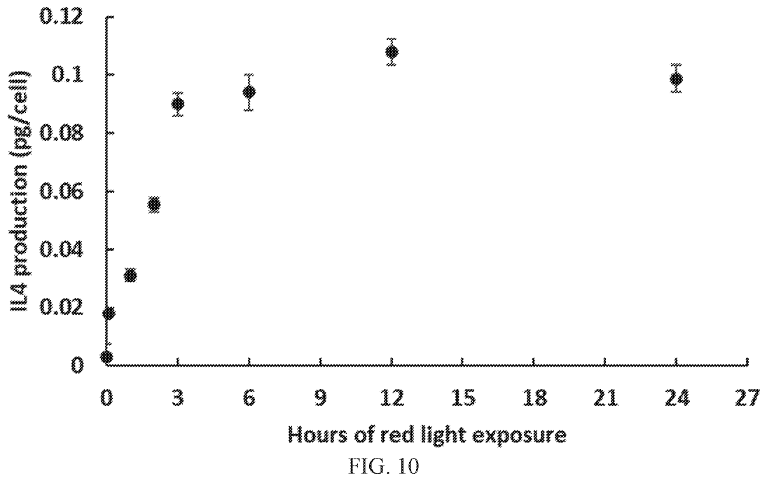

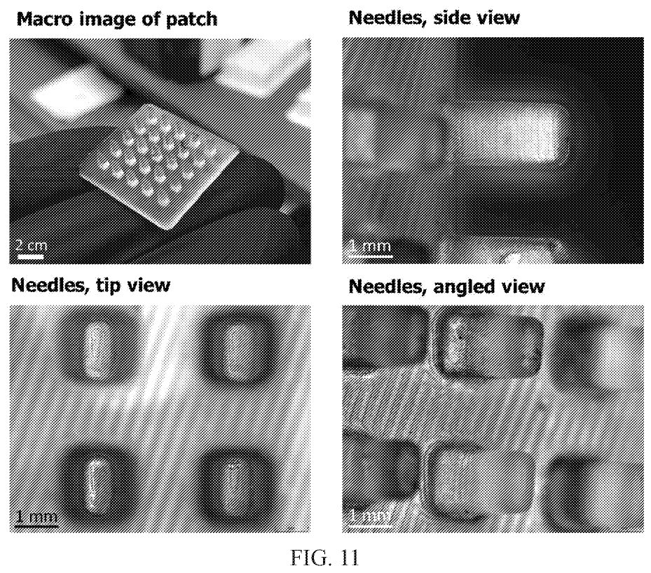

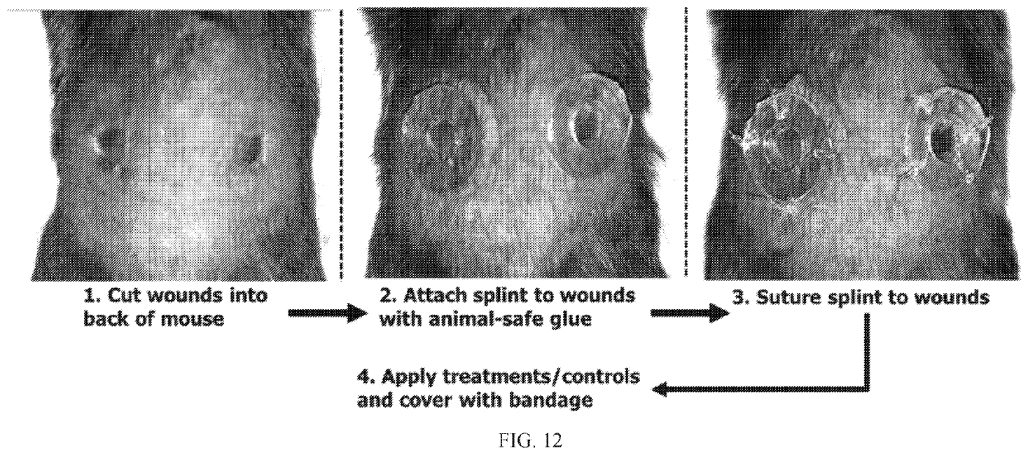

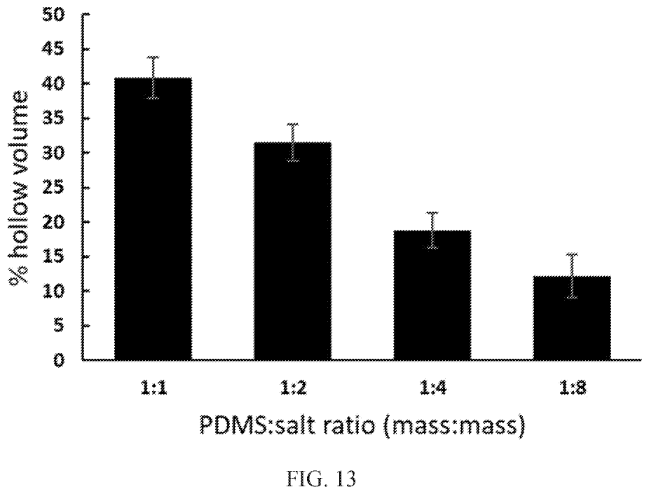

In embodiments of the invention, porous (i.e., partially hollow) PDMS microneedle patches were fabricated to facilitate diffusion of therapeutic factors to the wound site. Originally, molding PDMS with 3D printed molds was problematic due to the PDMS adhering to the molds after curing. To solve this, the molds were heated to 65° C. overnight prior to pouring the molds. The manner in which the molds aligned caused the backs of the needles to only be partially hollow. In yet other embodiments of the invention, porous PDMS was employed comprising alginate and cells. To fabricate porous PDMS, salt was added to PDMS while curing and then dissolved afterward (cf. FIG. 13). Sustained release without attenuation of wound-healing factors from optigenetically engineered cells inducible by red light was detected employing patches comprising porous (partially hollow) PDMS needles (cf. FIG. 10). In embodiments, cells were loaded in hydrogel and subsequently crosslinked in needle patches with semi-hollow backs (cf. FIG. 11). Further validation in a murine wound model was also investigated (cf. FIG. 12).

### X. ENUMERATED EMBODIMENTS

1. A wound healing device comprising a first component comprising a chamber for housing a cell or cells.
2 The wound healing device of embodiment 1, further comprising a cells disposed in said first component, such as between about 10 and 10,000,000 cells/ml of said first component.
3. The wound healing device of embodiment 2, wherein said living cells are engineered to secrete a growth factor, a cytokine, a lymphokine, chemokine or neurotropic factor or hormone.
4. The wound healing device of embodiment 3, wherein said living cells are engineered to secrete IL-4, BDNF, TNF-α, NGF, IL-12, IL-10, EGF, FGF-2, PDGF, VEGF, IL-1, IL-6, CTGF, GM-CSF, leptin, adiponectin, IP-10, NGF, IGF-1, dopamine, acetylcholine, fractalkine, HMGB1, IL-1β, IL-1RA, IL-2, sIL-2Rα, IL-5, IL-7, IL-8 (CCL8), IL-13, IL-15, IL-17A, IL-18, IFN-γ, IP-10 (CXCL10), MIG (CXCL9), MIP-1α (CCL3), MIP-1β (CCL4), MCP-1 (CCL2), M-CSF, Eotaxin (CCL11), active/latent TGF-β1, and/or lactic acid other metabolites for glycolysis.
5. The wound healing device of embodiments 1-4, wherein said living cells are mesenchymal stem cells, keratinocytes, fibroblasts, chondrocytes or retinal pigment epithelial cells.
6. The wound healing device of embodiments 1-5, wherein said first component comprises or forms a chamber or array, such as a needle array or a microneedle array.
7. The wound healing device of embodiment 6, wherein said array is formed from first and second components, said first component comprising a chamber for housing the cell or cells, and a second component providing structural support to said first component.
8 The wound healing device of embodiment 6, wherein only said first component forms said needle or microneedle array.
9. The wound healing device of embodiments 1-8, further comprising an immunomodulatory agent in either or both the first and second component to mitigate immune responses against the device when placed into contact with living tissue in a subject.
10. The wound healing device of embodiments 1-9, wherein said first component is comprised of a biocompatible material, such as a hydrogel, e.g., alginate, alginate-acrylamide, chitosan, alginate-gelatin, Hyaluronic acid, chondroitin sulfate, PEG, PEGylated fibronectin, or peptide gels and/or wherein the second component is comprised of structurally supportive biomaterials such as PDMS, polyimide, polyurethane, polyethylene or PTFE.
11. The wound healing device of embodiments 3-10, wherein said engineered cells secrete said growth factor, cytokine, lymphokine, chemokine, or peptide (a) constitutively, (b) in response to light, such as blue light, orange light, green light, violet light, near infrared or red light or (c) constitutively but secretion is increased in response to light, such as blue light, orange light, green light, violet light, near infrared or red light.
12. The wound healing device of embodiments 1-11, further comprising (a) a biomarker sensor to map wound healing and/or (b) an electrical stimulator.
13. The wound healing device of embodiments 1-12, wherein the biomarker sensor measures nitric oxide, a chemokine or a cytokine, e.g., TGF-β.
14. The wound healing device of embodiments 12 or 13, wherein said biomarker sensor provides a spatial map of healing in said wound, said sensor optionally being operably connected to a light emitting device.
15. The wound healing device of embodiments 12 or 14, wherein said electrical stimulator is a low impedance/high charge injection stimulator.
16. A method of treating a wound in a living subject comprising applying a wound healing device according to embodiments 1-15 to said wound.
17. The method of embodiment 16, wherein said engineered cells secrete said growth factor, cytokine, lymphokine, chemokine, or neurotropic factor or hormone constitutively.
18. The method of embodiment 16, wherein said engineered cells secrete said growth factor, cytokine, lymphokine, chemokine, or neurotropic factor or hormone in response to light, such as blue light, orange light, green light, violet light, near infrared or red light.
19. The method of embodiment 16, wherein said engineered cells secrete growth factor, cytokine, lymphokine, chemokine, or neurotropic factor or hormone constitutively but secretion is increased in response to light, such as blue light, orange light, green light, violet light, near infrared or red light.
20. The method of embodiment 18 or 19, further comprising subjecting said wound healing device or portion thereof to light.
21. The method of embodiments 16-20, wherein said wound healing device further comprises a biomarker sensor to map wound healing, such as a nitric oxide, a chemokine or a cytokine, e.g., TGF-β.
22. The method of embodiments 16-20, wherein said wound healing device further comprises an electrical stimulator.
23. The method of embodiments 16-20, further comprising a biomarker sensor that monitors wound healing, such as a nitric oxide sensor, and/or an electrical stimulator.
24. The wound healing device of embodiments 21 or 23, wherein said biomarker sensor provides a spatial map of healing in said wound, said sensor optionally being operably connected to a light emitting device.
25. The method of embodiments 22 or 23, wherein said electrical stimulator is a low impedance/high charge injection stimulator.
26. The method of embodiments 22, 23 or 25, further comprising applying an electrical current to said wound.
27. The method of embodiments 16-26, wherein said wound is a skin wound, a muscle wound, a penetrating wound, a closed wound, an open wound, muscle loss, or organ damage.
28. The method of embodiments 16-27, wherein said wound is a chronic/non-healing wound.
29. The method of embodiments 16-27, wherein said wound is a traumatic wound.
30. The method of embodiments 16-27, wherein said wound is a surgical wound.

### XI. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Culturing and Transfection of Exemplary Cell Lines for Inducible Secretion of Wound-Healing Factors

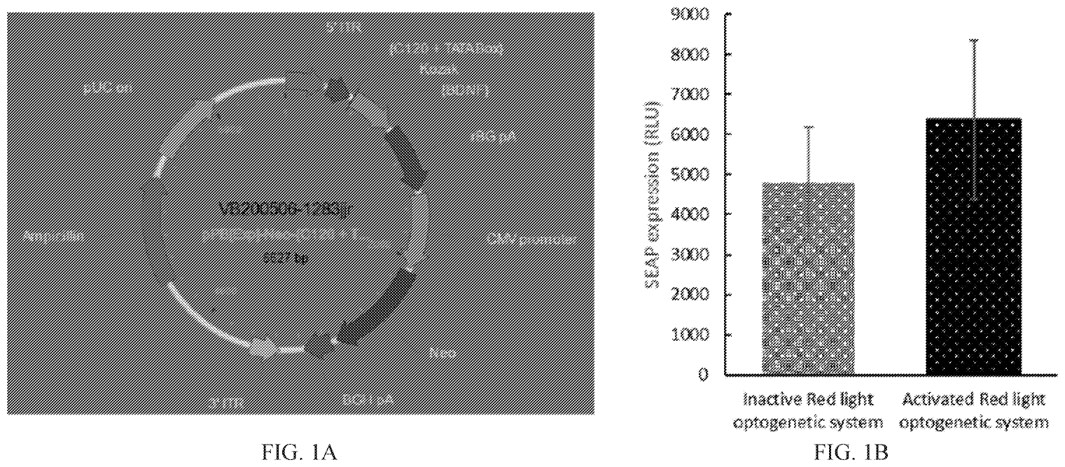

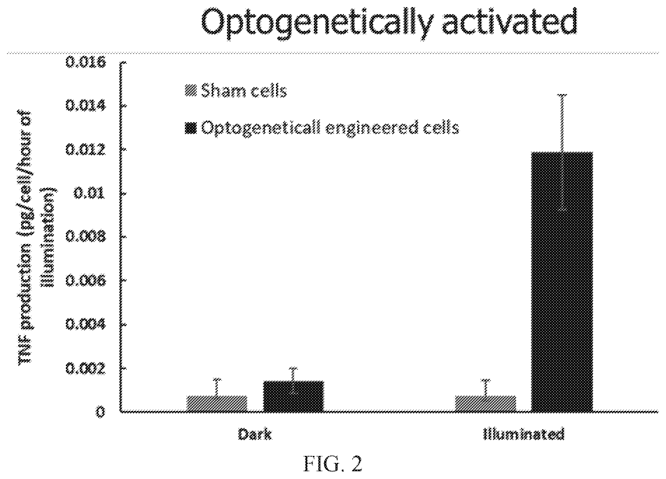

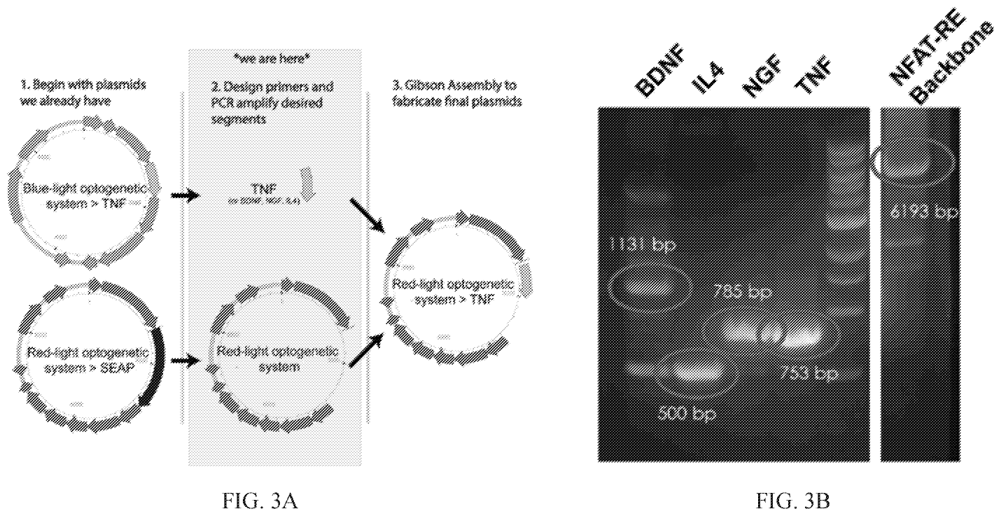

The example set forth below describes the culturing and transfection of exemplary cells with light-responsive systems of protein expression corresponding with FIGS. 1, 2, 6, 7, 8, and 10. Briefly, model retinal epithelial cells, ARPE-19, were cultured in media and transfected with Lipofectamine 3000 (ThermoFisher, Cat #L3000001) using ThermoFisher's standard protocol. Transfection of cell lines with light-responsive plasmids was tailored to achieve differential secretion of wound-healing factors depending on induction by various wavelengths of light. For blue-light responsive systems, plasmid 1 contained EL222, driven by the CAG constitutive promoter, while plasmid 2 contained the cytokine of interest, which was driven by the C120 promoter (promoter turned on by EL222). For red light responsive systems, plasmid 1 contained PhyB/PIF6 driven by the CAG constitutive promoter, while plasmid 2 contained the cytokine of interest being driven by NFAT-RE (promoter turned on by PhyB/PIF6). Stable cell lines were created with puromycin+neomycin selection. Unless stated otherwise, when performing in vitro validation experiments, cells were seeded in a 12 well plate at 500,000 cells per well (n=3 per condition). After a media change, cells were left in the dark for 24 hours and media was harvested. New media was added, and the same cells were then exposed to red (740 nm) or blue light (450 nm) for 24 hours, and media was subsequently harvested immediately. For experiments with PhyB/PIF6 cells, phycocyanobilin was also mixed in the media (the PhyB/PIF6's cofactor) to a final concentration of 15 μM to allow the system to be activated. Harvested media was analyzed via SEAP assay (Abcam, Cat #ab 133077) or via ELISA (Bio-techne, Cat #variable depending on cytokine analyzed).

### Example 2: Fabrication of PDMS/Alginate Patch

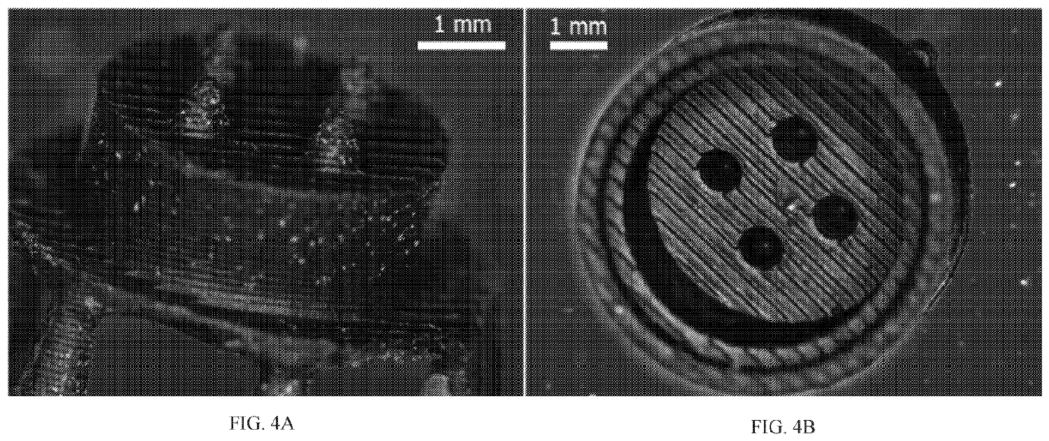

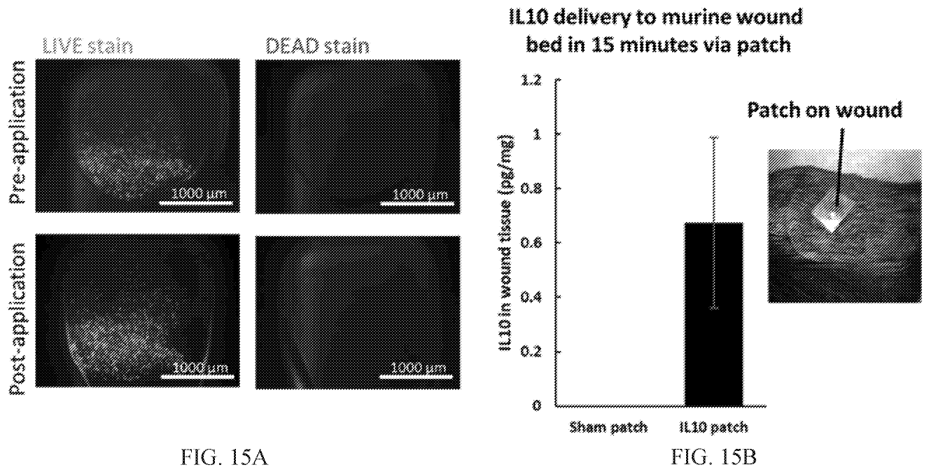

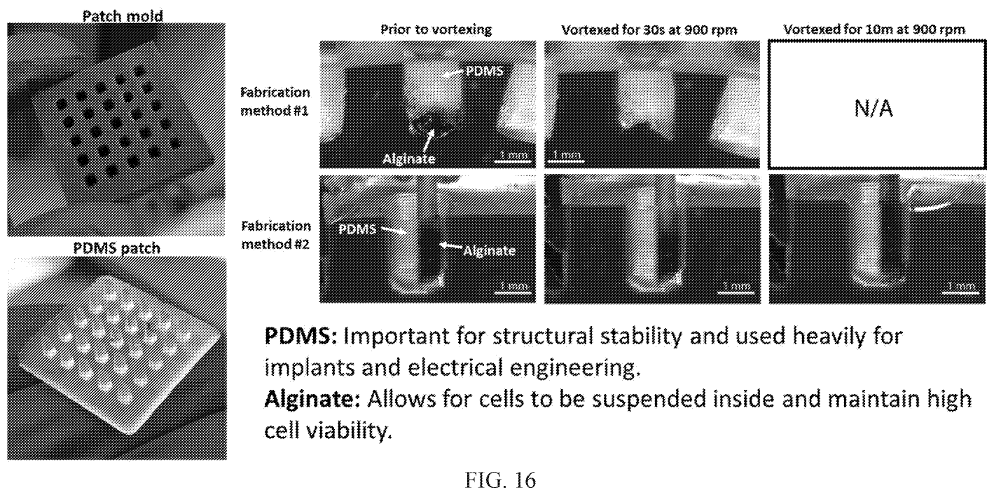

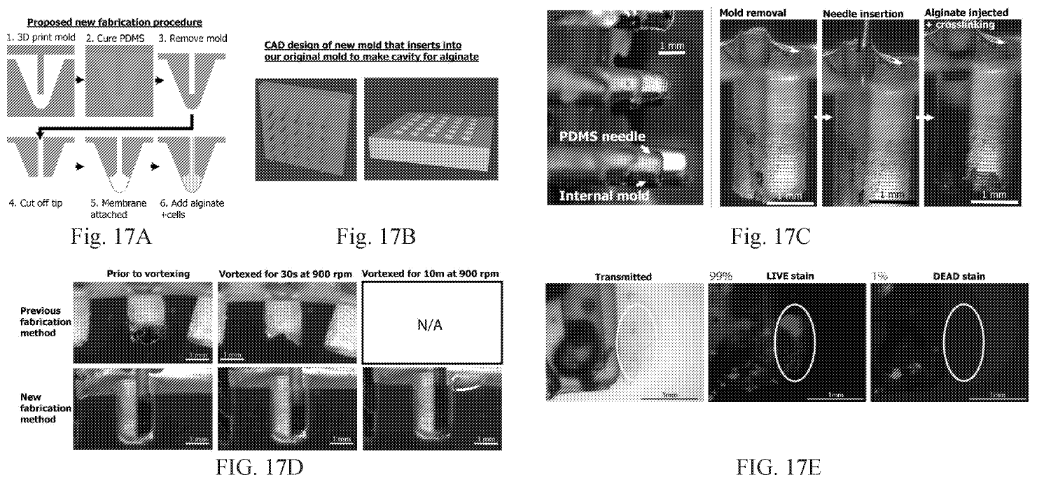

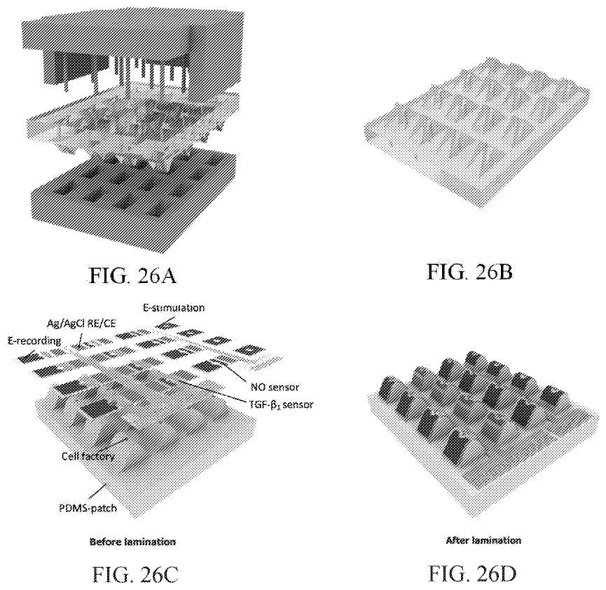

Recited below is a protocol delineating the fabrication of the PDMS/alginate patch employing a three-dimensional printer: the method of forming the top and bottom faces of the mold comprising resin, the method of preparing the PDMS elastomer, and the method of incorporating the cross-linked alginate comprising ARPE-19 cells into the needle tips or pillars of the patch (cf. FIGS. 4, 9, 11, 13, 14, 15, 16, 17 and 26).

Briefly, a Form2 three-dimensional (3-D) printer was used to print the top and bottom molds of the patch. Black V4 resin was employed for molds with a layer thickness setting of 25 microns. Rafts and supports were generated automatically. Immediately after the print finished and before removal from the baseplate, the molds were submerged into an isopropanol bath for 30 minutes. Molds were gently removed from the baseplate with a metal spatula and were submerged again in an isopropanol bath and vigorously swirled for 1 minute. Molds were subsequently brushed and cleansed with warm water to remove excess resin. The steps comprising submersion of the mold in the isopropanol bath and the brushing and cleansing steps with warm water were repeated twice for a cumulative total of three washes with isopropanol and water. Excess water was removed via flow of compressed nitrogen gas. Molds were placed in an oven at 60° C. overnight. The molds were optionally clamped to a plane (level) surface to mitigate mold deformation during the drying process when forming large molds (cf. FIG. 24).

PDMS silicone elastomer, e.g., the commercially available grade SYLGARD 184 from Dow® Chemical, was prepared by mixing the base and polymerizing agent in a 8:1 ratio. PDMS and polymerizing agent were stirred until the solution was an opaque, pearly white. The PDMS was then degassed in a vacuum chamber, and the PDMS solution was poured into the molds, such that that the level of the solution was congruent with the top of the mold. Excess bubbles were removed by allowing the PDMS to sit in the mold before curing. If air bubbles persisted a needle syringe was optionally employed for removal. PDMS was then allowed to cure at 65° C. overnight.

Thereafter, the cured PDMS was de-molded and cell-laden alginate was incorporated and subsequently crosslinked in the patch. Cell laden-alginate comprising 15 million cells/ml in 1.4% SLG20 alginate, wherein the cells are ARPE-19 cells engineered to secrete the indicated factor, was injected into the hollow chamber of the needle tips or pillars, and then submerged in crosslinking solution (CaCl₂)) for 10 minutes. Patches were then washed in phosphate-buffered saline (PBS) thrice and placed in media for storage in a cell culture incubator.

In embodiments of the invention the patch is optionally subsequently laminated with electrodes to provide electrical stimulation to areas of the wound site, to sense small molecules and proteins such as nitric oxide (NO) and TGF-β₁ or provide a combination of the aforementioned therapeutic modalities.

### Example 3: Murine Wound Model

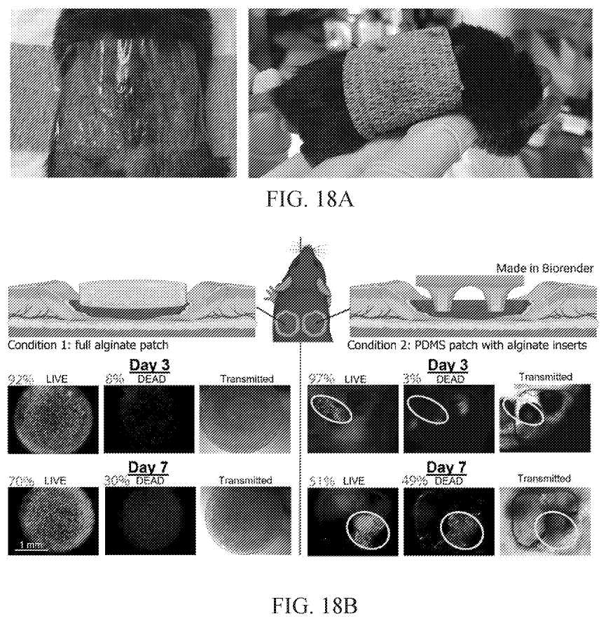

A protocol for a murine wound model for interrogating the efficacy of the wound-healing patch is delineated herein. Full thickness wounds were created on mice according to conventional methods familiar to one of skill in the art (cf. Murine Model of Wound Healing-PMC accessed from: https://www.nih.gov; Dunn L, Prosser H C, Tan J T, Vanags L Z, Ng M K, Bursill C A. Murine model of wound healing. J Vis Exp. 2013 May 28; (75): e50265. doi: 10.3791/50265. PMID: 23748713; PMCID: PMC3724564). After wound creation patches fabricated by methods recited in Example 2 were first applied over the wound, followed by placement of Tegaderm™, a transparent film dressing available from the 3M Company, over the entirety of its back. Then a self-adhesive bandage over the Tegaderm™ was wrapped around the mouse to secure the aforementioned wound dressings (see FIGS. 12, 15, and 18).

In order to interrogate the delivery of cytokines to the wound bed, the wound tissue was extracted and homogenized. Briefly, about 30 mg of each sample was trimmed and weighed to record a weight for each sample. Then 400 microliters of extraction buffer (Tissue Protein Extraction Buffer, T-PER™ available from Thermo-Fisher Scientific, and protease mixture) was added to each sample tube. Samples were subsequently homogenized with an electric homogenizer for 30 seconds on ice. 400 microliters of extraction buffer were used to wash the tips of the homogenizer into the sample tube. The tip of the homogenizer was then washed with ethanol, water, and PBS. Between liquids the homogenizer was allowed to run in an empty beaker to expel excess liquid remaining on the homogenizer tip. Samples were then centrifuged for 20 minutes at 13,000 rpm in a tabletop centrifuge at 4° C., and the supernatant was collected and stored at −80° C. for later analysis.

### Example 4: Cell Viability Assay and Fluorescence Microscopy Imaging

Provided below is a method for determining the viability of cells incorporated in the hollow chambers of needles or pillars in the wound-healing patch. A standard cell viability assay was followed, namely the LIVE/DEAD Viability/Cytotoxicity Kit for Mammalian Cells which is commercially available from Thermo Fisher Scientific. In short, patches fabricated in Example 2, which were loaded with ARPE-19 cells in crosslinked alginate, were incubated in staining solution containing 5 μL of calcein AM and 20 μL ethidium homodimer-1 in 10 mL Dulbecco's Phosphate-Buffered Saline (DPBS) for 30 minutes. Cells were then imaged on an EVOS fluorescence microscope. A green color indicated the presence of live cells.

### Example 5: Gibson Assembly Method for Cloning of Optogenetic DNA Constructs

Described below is a method to clone photoinducible plasmid systems de novo from individual DNA fragments. The Gibson Assembly protocol, which is accessible from New England BioLabs (available at: https://www.neb.com/protocols/2012/12/11/gibson-assembly-protocol-e5510), is well-known to those of skill in the art and prescribes the following in brief: firstly, pipette 0.02-0.5 pmol DNA fragments together with 10 μl reaction mix, Gibson Assembly Master Mix™, and balance DI H₂O to a total volume of 20 μl, in conjunction with a positive control mixture; second, place reaction mixtures in a thermocycler for polymerase chain reaction (PCR) amplification for 15 minutes at 50° C. (instances with two or three (2-3) DNA fragments) or 60 minutes (instances with four to six (4-6) DNA fragments); third, store reaction products at −20° C. prior to transformation; and finally, transform NEB 5-α Competent E. coli cells as delineated per kit instructions.

### Example 6: In Vitro Characterization of Red-Light Responsive Optogenetic Cells

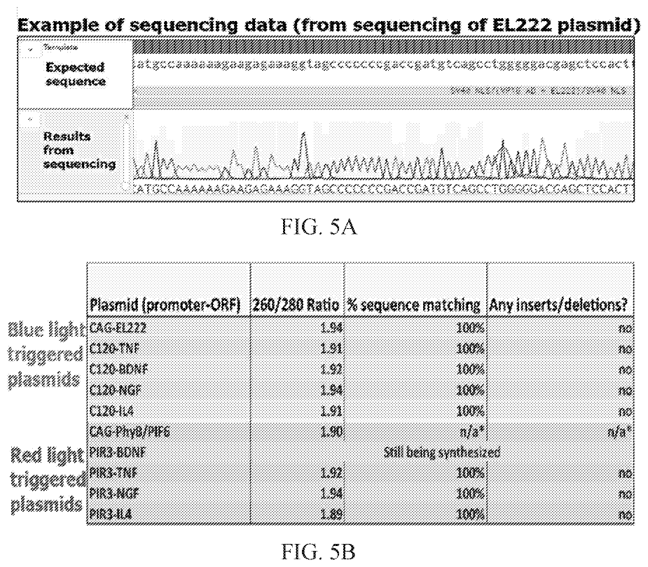

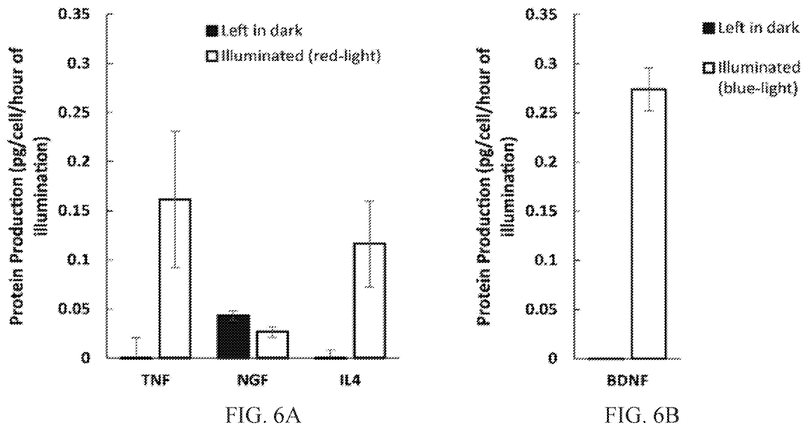

The example set forth below describes characterization of optogenetic cells responsive to red light that inducibly secrete BDNF, TNF-α, NGF, and IL4, alone or in combination. Plasmids containing BDNF, TNF-α, NGF, and IL4 under control of an optogenetic system were designed and subsequently ordered from VectorBuilder. VectorBuilder provided the fully synthesized plasmids containing BDNF and NGF genes, respectively. An increased expression of the SEAP reporter protein was observed upon induction with red light as shown in FIG. 1B. Primer design and PCR amplification of the components to fabricate plasmids that have the PhyB/PIF6 red-light optogenetic system for driving expression of the therapeutic proteins has also been confirmed by sequencing analysis (cf. Example 1 and FIG. 5). The cells comprising the PhB/PIF6 red-light optogenetic system have a secretion rate above 0.1 pg/cell/hour of illumination (cf. FIG. 6).

### Example 7: In Vitro Characterization of Blue-Light Responsive Optogenetic Cells

The example set forth below describes characterization of optogenetic cells responsive to blue light that inducibly secrete BDNF, TNF-α, NGF, and IL4, alone or in combination. Plasmids containing BDNF, TNF-α, NGF, and IL4 under control of the blue-light optogenetic system, EL222, were synthesized following cloning methods as described previously herein (cf. Example 1). The EL222 blue-light triggered optogenetic system was used to drive therapeutic protein production at a rate of 0.012 pg/cell/hour of light (see FIG. 6). Likewise, TNF-α expression was triggered by blue light in the optogentically engineered cells at similar rates.

### Example 8: Optimization of Patch Microneedle Design, Viability of Optogenetic Cells in Crosslinked Alginate-Acrylamide, and Secretion of Wound-Healing Factors

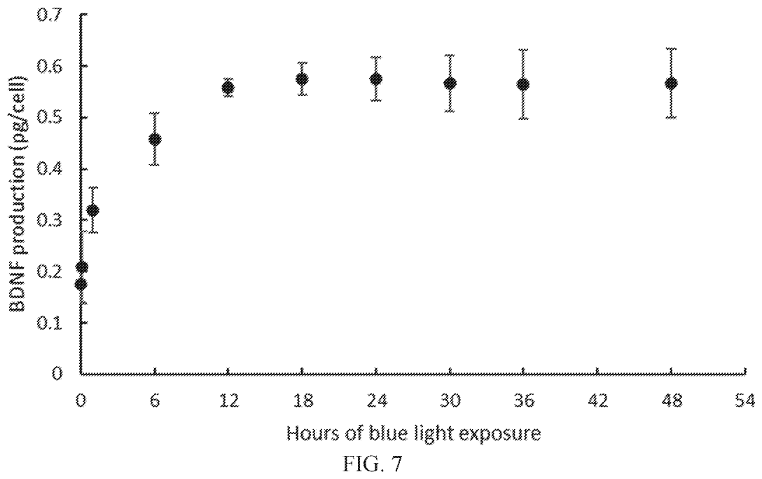

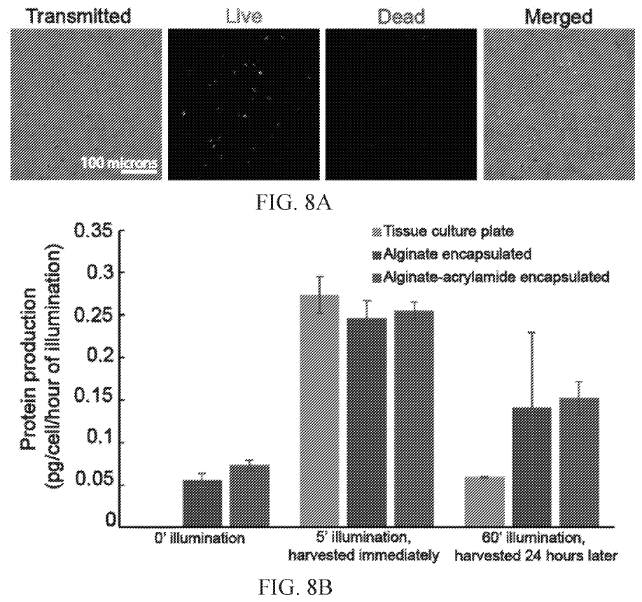

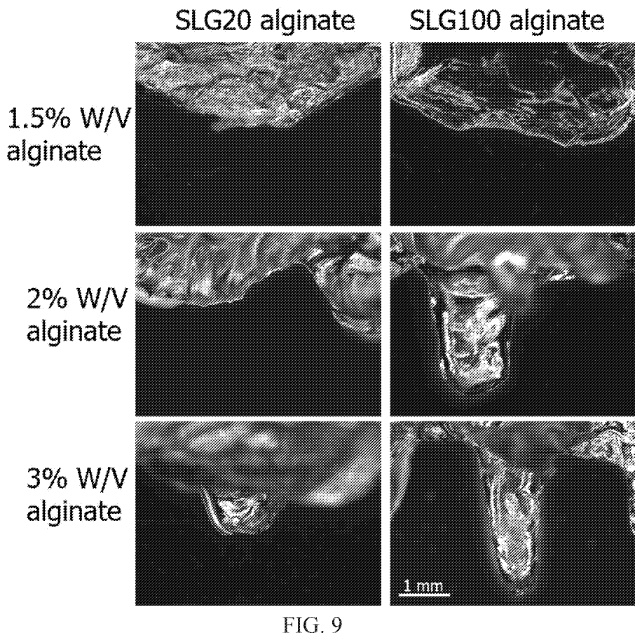

Optimization of the microneedle design, viability of optogenetic cells in crosslinked hydrogel, and detection of healing factors diffusing into the wound site are described herein. The engineered cells maintain high viability in covalently crosslinked alginate-acrylamide, an embodiment of the inventive hydrogel microneedle patch, as demonstrated by cell viability/cytotoxicity assays (see FIGS. 8A-8B). Additionally, encapsulating the cells in alginate-acrylamide did not negatively affect their BDNF production rate (cf. FIG. 7).

Conditions were systematically varied to optimize microneedle patch fabrication. Original formulation of alginate-acrylamide hydrogels did not consistently form the microneedles. To solve this, the alginate concentration was modulated. It was found unexpectedly that a higher concentration of alginate caused better microneedle formation compared to former embodiments, as shown in FIG. 9.

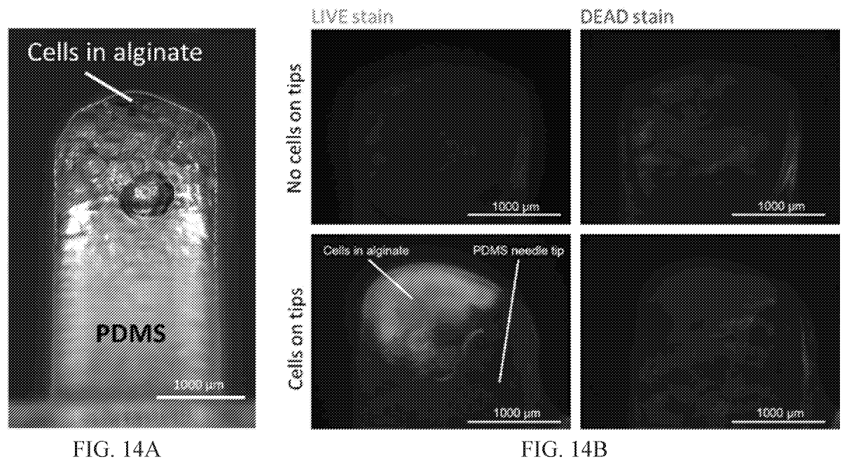

Sustained release without attenuation of wound-healing factors from the optogenetically engineered, light-triggered cells was observed. Secretion of healing factors from cells cultured in covalently crosslinked alginate-acrylamide was measured by enzyme-linked immunoassay (ELISA) (previously described in Example 1; see also FIG. 6) as is well-known in the art. Delivery of wound healing factor (IL10) into the murine wound model was detected after 15 minutes of application (see FIG. 14; see also 20, 22, 23).

### Example 9: Fabrication of Porous (Partially Hollow) PDMS Microneedle Patches

A method of fabrication of porous PDMS microneedle patches are described in the example below. In embodiments of the invention, porous (i.e., partially hollow) PDMS microneedle patches were fabricated to facilitate diffusion of therapeutic factors to the wound site. Originally, molding PDMS with 3D printed molds was problematic due to the PDMS adhering to the molds after curing. To solve this, the molds were heated to 65° C. overnight prior to pouring the molds. The manner in which the molds aligned caused the backs of the needles to only be partially hollow. In yet other embodiments of the invention, porous PDMS was employed comprising alginate and cells. To fabricate porous PDMS, salt was added to PDMS while curing and then dissolved afterward (cf. FIG. 13). Sustained release without attenuation of wound-healing factors from optigenetically engineered cells inducible by red light was detected employing patches comprising porous (partially hollow) PDMS needles (cf. FIG. 10). In embodiments, cells were loaded in hydrogel and subsequently crosslinked in needle patches with semi-hollow backs (cf. FIG. 11). Further validation in a murine wound model was also investigated (cf. FIG. 12).

### Example 10: Macrophage Transcriptome Analysis of Wound Bed After Cytokine Exposure

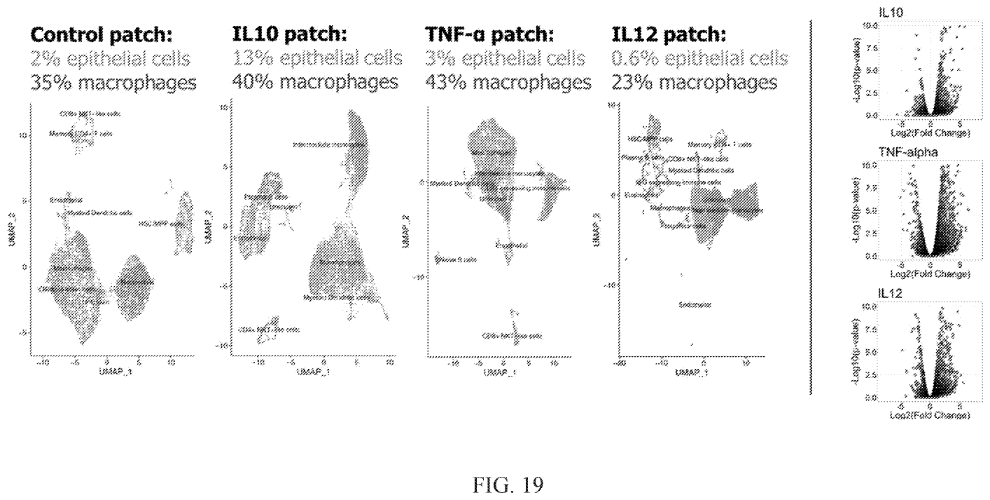

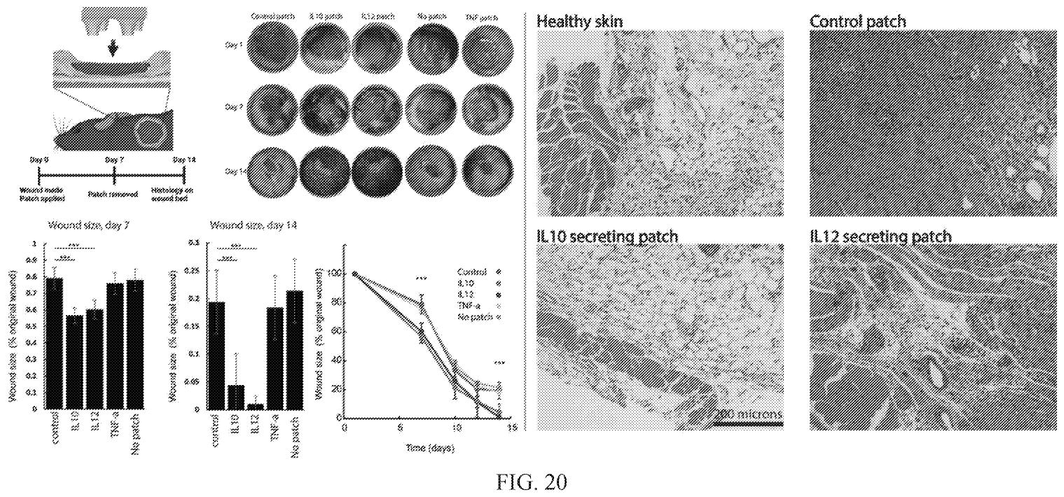

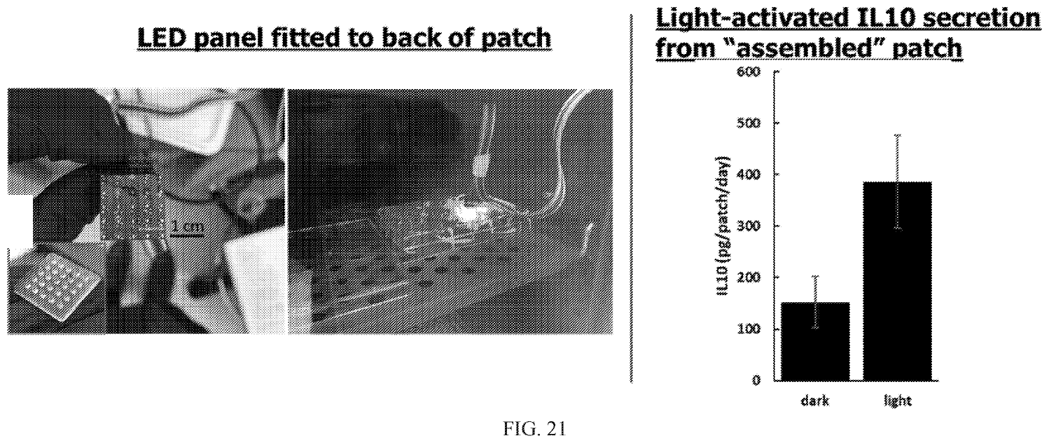

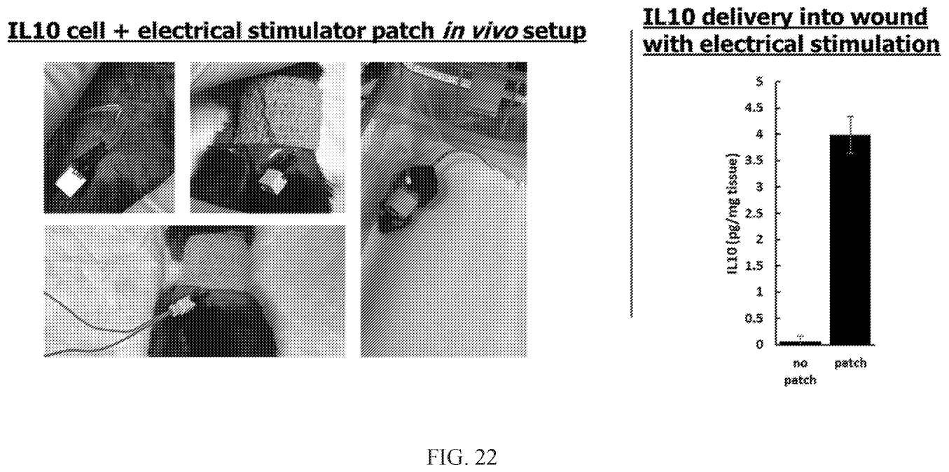

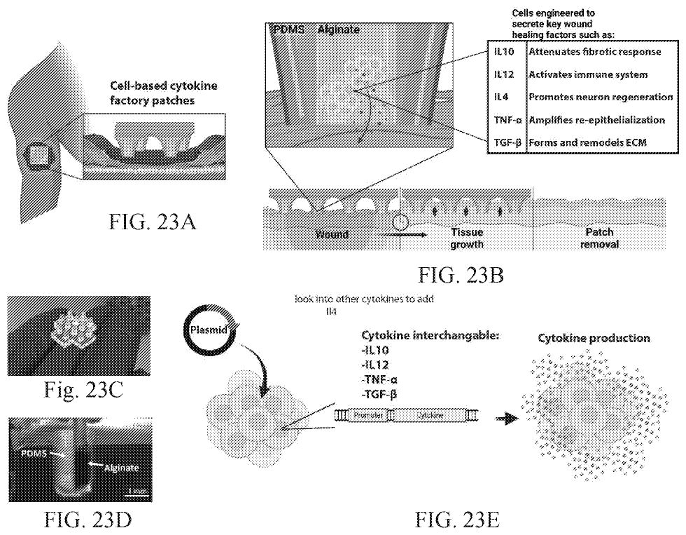

The following protocol describes a method to determine the transcription levels of immune cells in extracted murine wound tissue that has been treated with wound-healing factors and or cytokines, as shown in FIG. 19. After wounds were created, patches were added to the wound bed and left for four days with optogenetically engineered cells expressing IL10, IL12, and TNF-α. Controls were also included comprising unengineered cells and wound with no patch. After patch removal, wounds were removed, and a single cell suspension was created. Mice were euthanized via CO₂ euthanasia under isoflurane anesthesia. Wounds were excised, keeping ˜1 mm of tissue around the wound. Tissue was minced and put into tubes of media with Liberase™ (1.2 ml DMEM NO FBS OR ANTIBIOTICS, with 60 μg/ml liberase). The tissue was incubated for 1.5 hours at 37° C. in a rotator. Samples were moved to homogenizer tubes (gentleMACS C tubes available from Miltenyi Biotec), with all five wounds from a condition pooled together. Tissues were physically homogenized with the gentleMACS tubes with the human-tissue setting 3× for each tube. Tubes were alternated so that no tube is off ice for a substantial period. The fluid in the tubes was then poured through a 70 μm cell strainer into a new 50 ml conical tube. The fluid was centrifuged in a refrigerated centrifuge for 5 min at 300 g, 4° C., the supernatant was removed, and the cells were suspended in 5 ml RBC lysis buffer for 5 minutes. 25 mL of ice-cold DPBS was added to quench the lysis buffer, and the cells were subsequently spun down for 5 mins at 300 g, 4° C. The supernatant was discarded, and cells were gently resuspended in ice-cold DPBS media by gently tapping the bottom of the tube and pipetting up and down. Cells were counted using a 1:1 ratio of trypan blue to cell stock. Cells were then spun down for 5 mins at 300 g, 4° C. and resuspended in sorting buffer with the dead cell stain Propidium Iodide (15 million cells/mL max for the sorter). The “Propidium Iodide Ready Flow Reagent” from Invitrogen was used (R37169), following the standard protocol as provided by the vendor. The sorting buffer used was: 500 ml 1×DPBS, 12.5 mL IM HEPES, 5 mL FBS, and 5 mL 0.5M EDTA. FACS analysis was used to sort the dead cells. Cells were taken to Baylor College of Medicine core for library prep and sequencing.

### Example 11: Synthesis and Electrochemical Characterization of Bare and Polymer Coated Metal Electrode Arrays

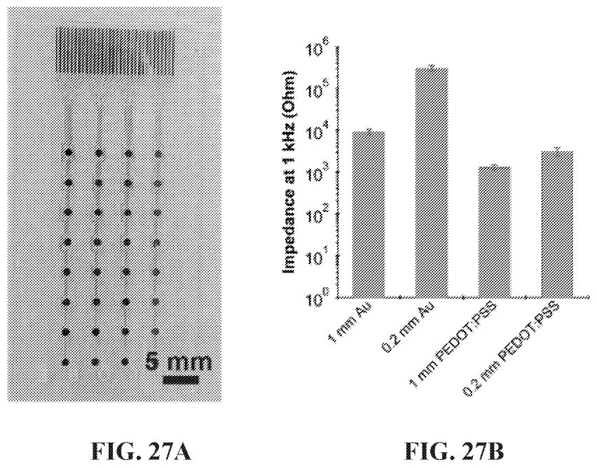

In this example, electrode arrays comprising gold electrodes of 0.2 and 1 mm in diameter were synthesized by photolithography under clean room conditions and imaged by optical microscopy as shown in FIG. 27A. Impedance measurements of both the clean metal electrodes and metal electrodes coated in Poly(3,4-ethylenedioxythiophene)-polystyrene sulfonate (PEDOT:PSS) are shown in FIG. 27B. For electrode surfaces of the same size, coating with PEDOT:PSS decreases electrical impedance compared to a bare metal surface. Electrochemical impedance provides a direct estimate of the recording capabilities of an electrode. Designing bioelectrical interfaces with low impedances is important for enhancing the signal-to-noise ratio during electrophysiology recordings.

### Example 12: Electrochemical Characterization of PEDOT:PSS Electrodeposited Electrodes

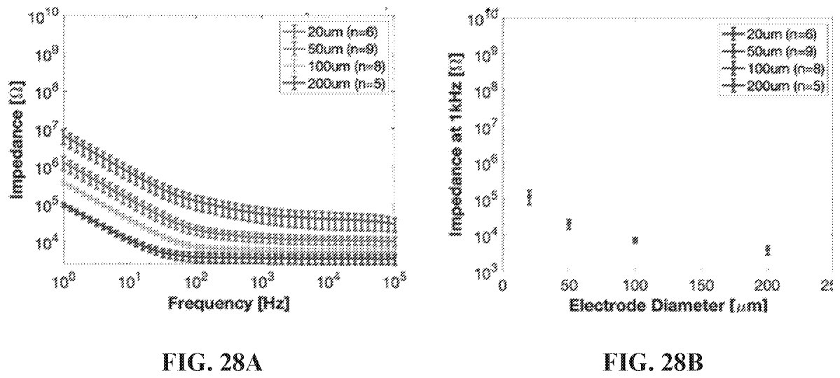

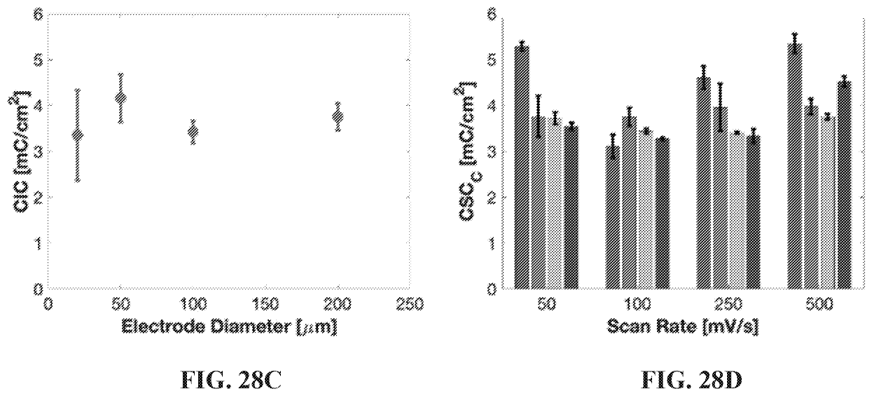

In this example, electrodes of various sizes were synthesized, coated with PEDOT:PSS and evaluated for electrochemical characteristics. As shown in FIG. 28A-B, the electrochemical impedance of all microelectrodes decreased as the diameter of the electrodes was increased from 20 μm to 200 μm. Increasing the diameter of the electrodes increases the electrode area, which leads to both decreased resistive and increased capacitive behavior of the electrodes; thus, reducing the overall electrode impedance. The frequency of electrophysiological signals varies widely depending on the target cells and tissues. For example, local field potentials (LFPs), accumulation of electrical activity of multiple neurons, typically have frequencies less than 300 Hz. Similarly, intracellular oscillations at frequencies less than 1 Hz have also been reported in cortical neurons. To successfully record such electrophysiological signals, the interfaced bioelectronics must exhibit low electrochemical impedance in the low-frequency regime.

Electrical stimulation is generally achieved through as a series of biphasic current pulses with cathodal and anodal phases. To prevent damage of biological structures during electrical stimulation, the maximum cathodic potential drop (E_{mc}) and the maximum anodic potential drop (Eₘₐ) across the electrode-electrolyte interface should not exceed the electrolysis window for H₂O under any stimulating current pulse. The magnitude of the stimulation current pulses that can be safely applied at the electrode-electrolyte interface is governed by the charge injection capacity (CIC) of the microelectrodes. Therefore, CIC is determined as the amount of charge that can be injected to the electrode without crossing E_{mc} and is assessed through voltage transient measurements. The CIC values of PEDOT:PSS-coated gold electrodes of varying sizes are shown in FIG. 28C.

Ideal electrical stimulators should exhibit capacitive response rather than faradaic to avoid electrolysis of media, oxidation of metabolites, and maintain a stable electrode-electrolyte interface. To avoid electrolysis of H₂O in the aqueous media, the potential window in cyclic voltammetry (CV) scans should be maintained within the water electrolysis window. The potential windows for safe operation of the electrodes without electrolysis of H₂O were determined to be −0.8 to +0.8. By computing the time integral of the cathodic regime of the CV scans of the microelectrodes, we determined the cathodic charge storage capacity (CSC_{C}) of each electrode material as shown in FIG. 28D. PEDOT:PSS based microelectrodes exhibit primarily capacitive charging with CSCC much greater than metallic microelectrodes of similar sizes

### Example 13: Stimulation of a Rat Sciatic Nerve with a Custom Flexible Stimulation Array

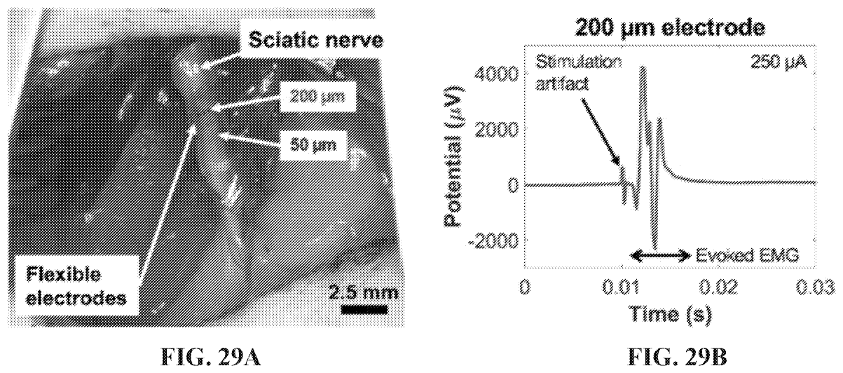

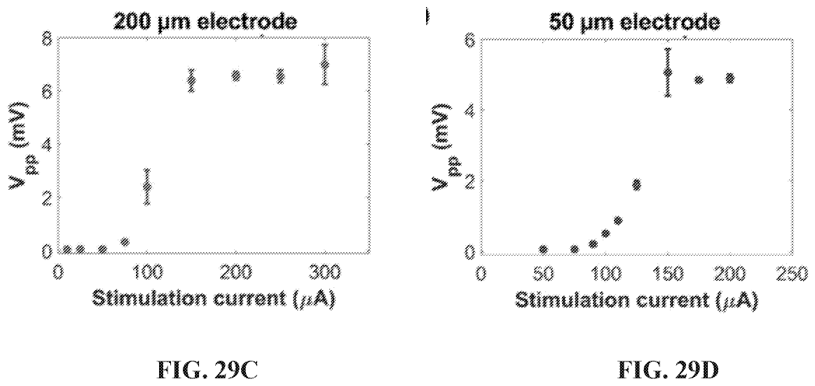

In this example, Au electrode arrays coated in PEDOT:PSS were used to evoke skeletal muscle contraction by stimulating the sciatic nerve in an anesthetized rat subject. The placement of 50 and 200 μm electrode arrays on the rat sciatic nerve was achieved as shown in FIG. 29A. Evoked skeletal muscle activity (EMG) was recorded with a stainless-steel wire inserted into distal muscle; a representative voltage vs. time plot for a 200 μm electrode under applied 250 μA current is shown in FIG. 29B. The EMG responses at increasing currents create recruitment curves, which are used to visualize the progressive recruitment of myofibers in the muscle. As shown in FIGS. 29 C-D, the recruitment curves for both 50 and 200 μm electrodes plateau at approximately 150 μA, the point at which all myofibers contained in the sciatic nerve are firing. This result demonstrates the stimulation potential of our arrays with electrodes of various sizes.

### Example 14: Demonstration of Mapping of VML Wound Bed Electrophysiology

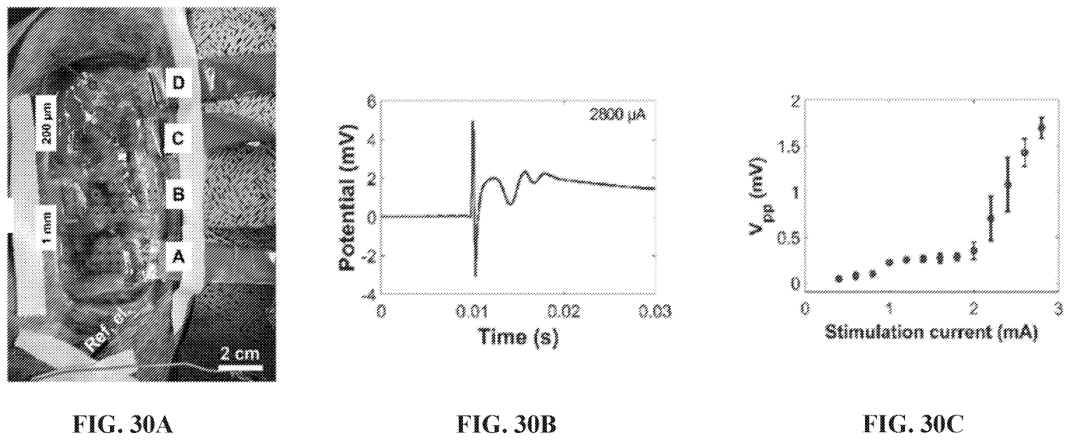

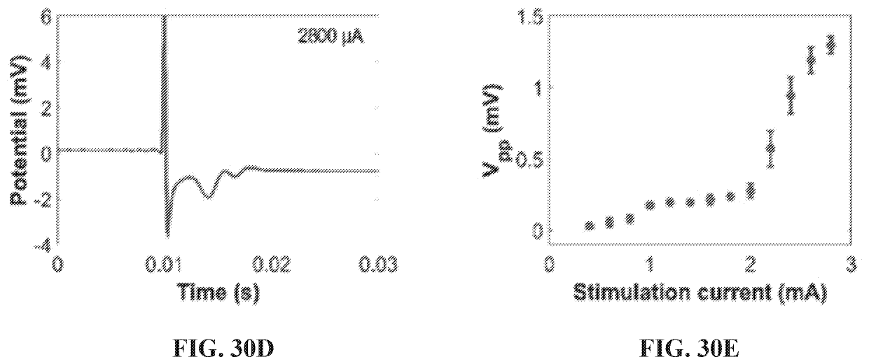

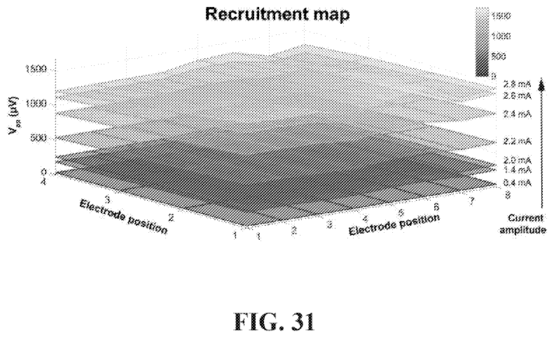

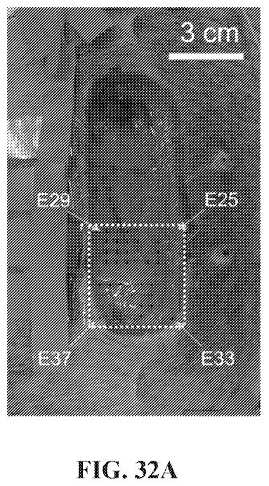

In this example, Au electrode arrays coated in PEDOT:PSS were used to record skeletal muscle contraction in a volumetric muscle loss (VML) wound in a canine subject. As shown in FIG. 30A, multielectrode arrays comprising 1 mm (“A” and “B” in the image) and 200 um (“C” and “D” in the image) electrodes were adhered to the open wound of the subject. The sciatic nerve was stimulated with a commercial nerve cuff and the EMG responses for the 200 um and 1 mm electrode sizes were recorded and recruitment plots were produced (FIGS. 30B-E). This demonstrates the recording potential of our custom electrode arrays on a wound bed.

### Example 15: Demonstration of Mapping of Muscle Recruitment Curve Using High-Density Electrode Arrays

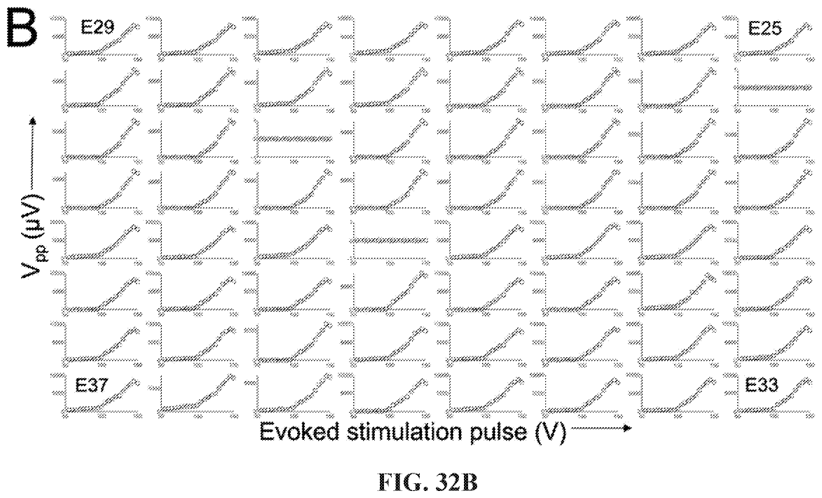

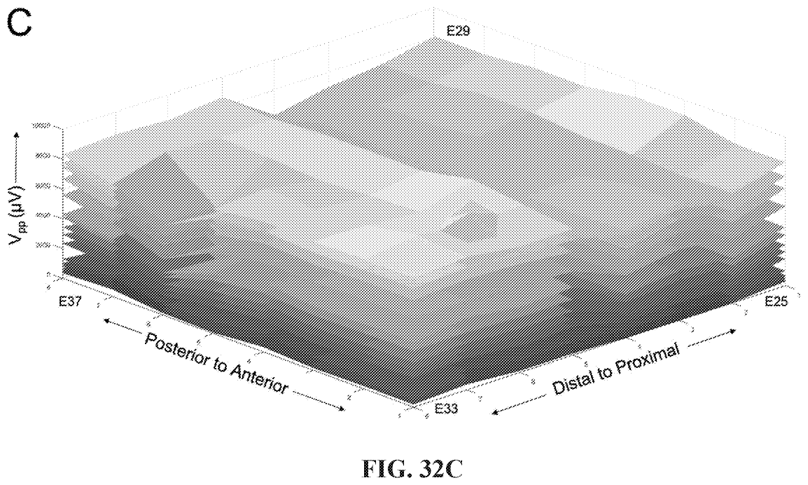

In this example, the feasibility of mapping evoked EMG responses for both sciatic nerve and open-wound models was evaluated. An electrode array comprising 32 PEDOT:PSS electrodes was applied to the sciatic nerve of a rat subject and recruitment curves were collected from 0.4-2.8 mA currents (FIG. 31). These results were extended to a canine VML wound model using an electrode array comprising 64 PEDOT:PSS electrodes (FIG. 32A-C), further supporting the feasibility of evoked EMG mapping as a metric for VML regeneration. To our knowledge, this is the first-time progressive stimulation and evoked CMAP recordings from the same VML wound bed have been quantified. This result demonstrates the ability to simultaneously evoke skeletal muscle contraction on the wound bed (proximally) and record skeletal muscle contraction (distally).

### Example 16: Patch Fabrication for Electrical and Bio-Chemical Actuator Integration

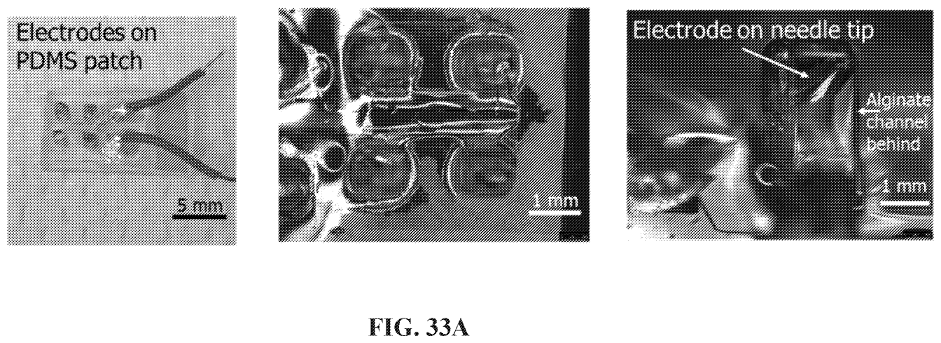

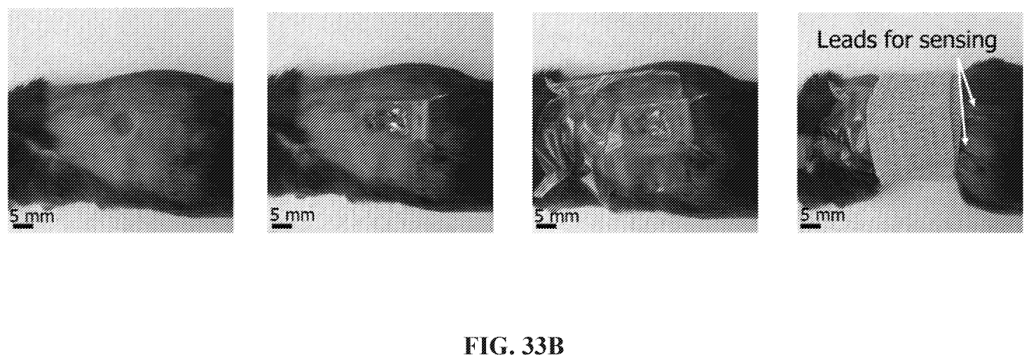

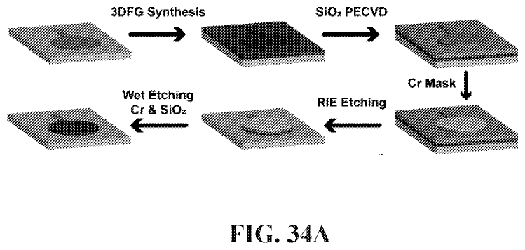

In this example, the feasibility of combining electrical stimulation and biochemical actuation in a wound healing patch is demonstrated. As shown in FIG. 33A-C, a polydimethyl siloxane patch was fabricated that can house both the electrical and cellular components. As shown in FIGS. 33D-G, the patch can be easily applied to a mouse wound model by first applying a Tegaderm™ wrap followed by a conventional bandage wrapping.

### Example 17: Synthesis of High Surface Area Three-Dimensional Fuzzy Graphene (3DFG) Electrodes

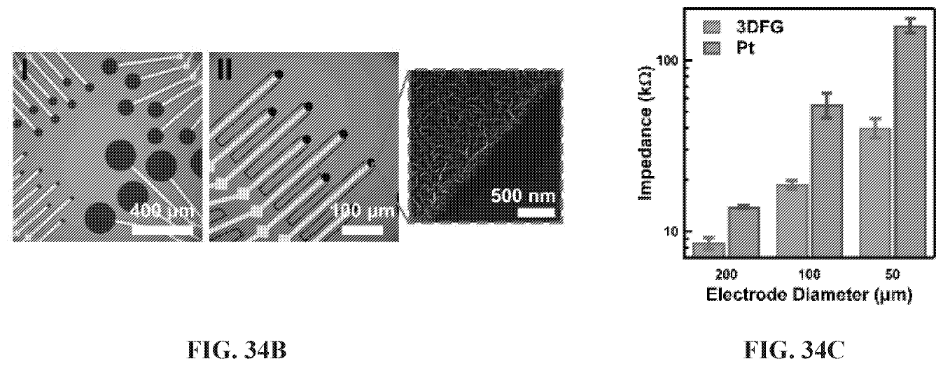

In this example, another electrode material, three-dimensional fuzzy graphene (3DFG), is synthesized. 3DFG is a high surface area nanostructured material that can be used for electrodes of the wound healing devices described herein. 3DFG was synthesized according to the method of FIG. 34A and verified by optical microscopy and scanning electron microscopy (FIG. 34B). Impedance analysis shows that impedance is decreased for 3DFG electrodes compared to platinum electrodes of the same size (FIG. 34C).

### Example 18: Electro-Polymerization of Ni-TMHPP on 3DFG Multi Electrode Arrays

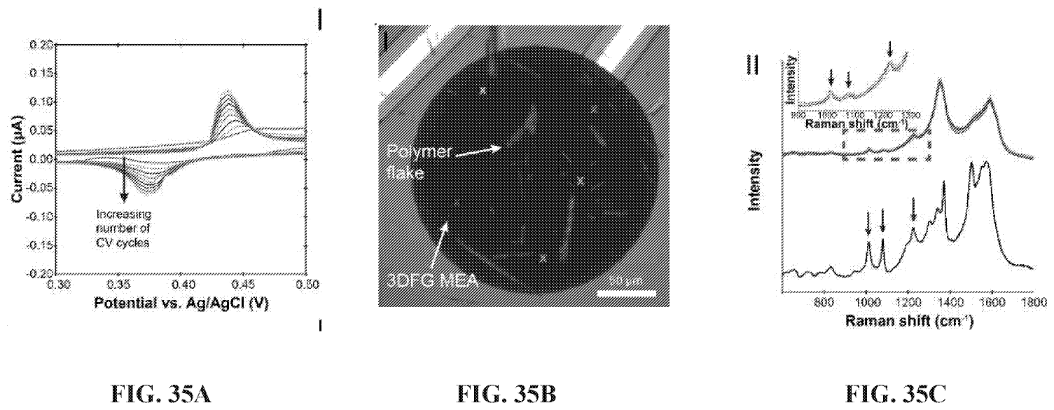

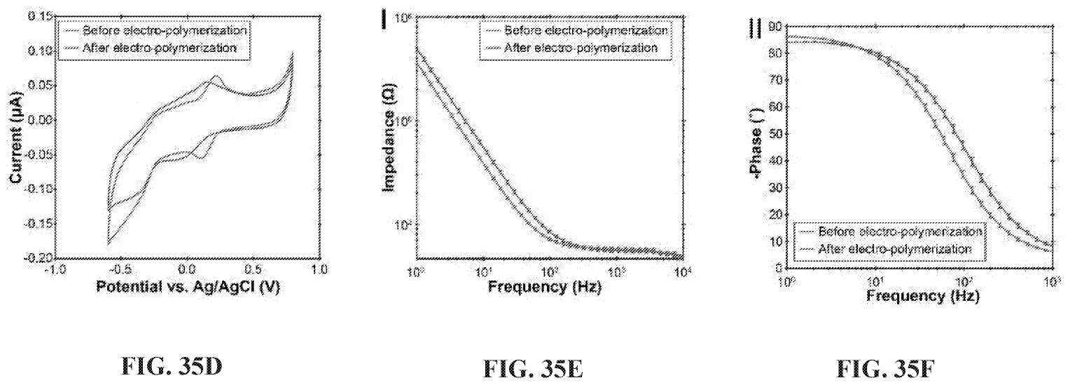

In this example, a metal catalyst is added to the 3DFG surface to catalyze the NO oxidation reaction. Nickel-tetrakis(3-methoxy-4-hydroxy-phenyl) porphyrin (Ni-TMHPP) was deposited on the surface of 3DFG by electro-polymerization according to established protocols and verified by cyclic voltammetry (FIG. 35A). As shown in FIG. 35B various regions of the prepared film were analyzed by and Raman spectroscopy (FIG. 35C) to confirm that electro-polymerization occurred The materials were further characterized by cyclic voltammetry and electrochemical impedance spectroscopy pre- and post-modification (FIGS. 35D-F).

### Example 19: Electrochemical Sensing of NO Via Square Wave Voltammetry Method

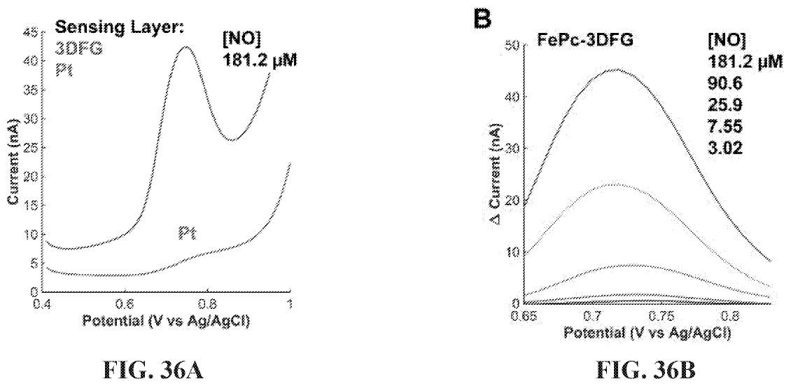

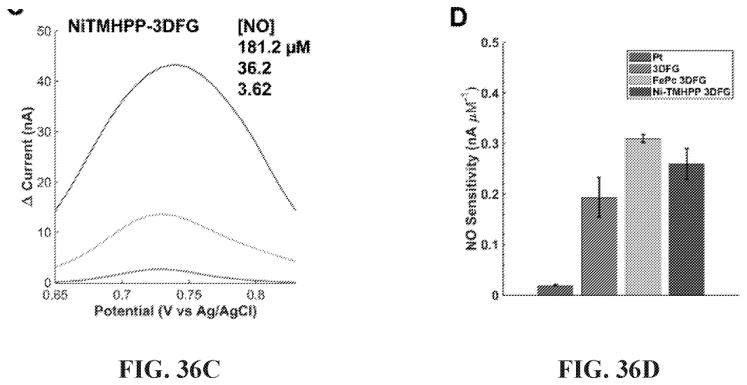

In this example, the ability of different bare and modified substrates to detect nitric oxide were assessed by square wave voltammetry. As shown in FIG. 36A, 3DFG exhibited a higher current response towards NO oxidation compared to Pt. The detection of NO by 3DGF modified with iron phthalocyanine (FePc) and 3DGF modified by Ni-TMHPP were evaluated as shown in FIGS. 36B-D. The presence of these catalyst compounds on the 3DFG lead to an increase in sensitivity towards nitric oxide.

### Example 20: Electrochemical Sensing of NO Via Chronoamperometry Method

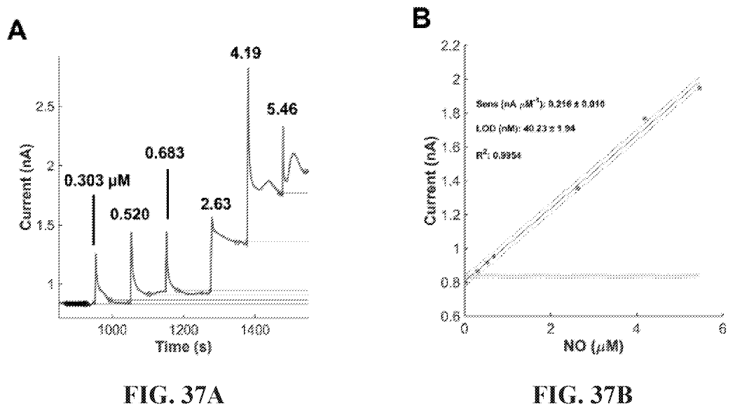

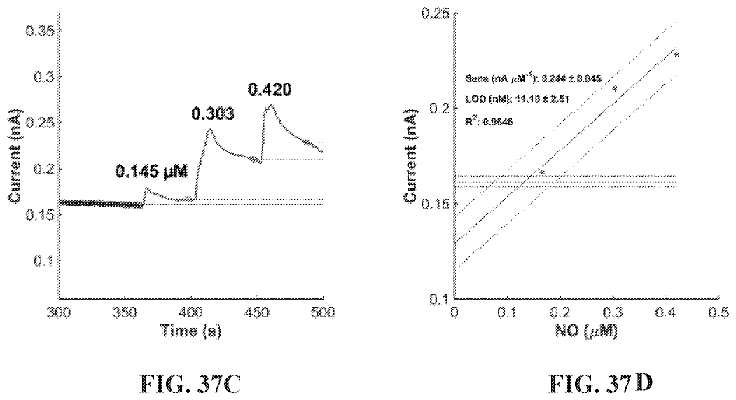

In this Example, the ability of the 3DFG electrodes of Example 19 were used to detect NO using chronoamperometry. As shown in FIG. 37A, standard NO solutions were prepared and peak current readings were recorded. The peak current reading and cumulative nitric oxide concentration were analyzed by linear regression. Slope is the sensitivity of the electrode.

### Example 21: Synthesis of Semipermeable Nafion Coatings for Selective Electrochemical Sensing of NO

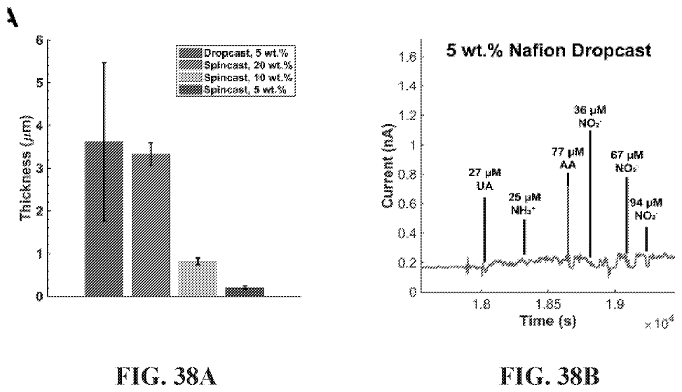

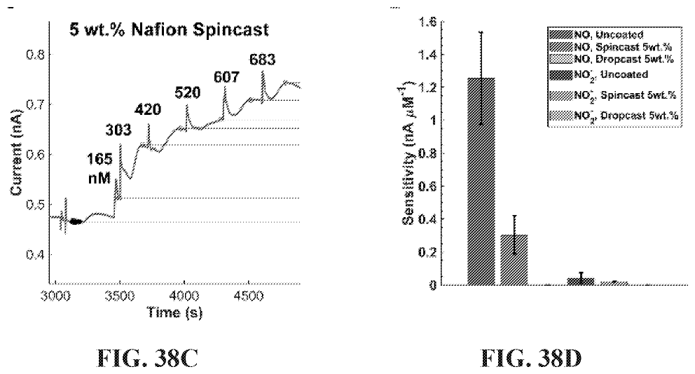

In this example, the 3DFG electrodes of Examples 19 were modified with a Nafion coating. Nafion is a fluorinated polymer coating that serves as a selective layer for nitric oxide sensing. As shown in FIG. 38A, the thickness of different coating techniques including dropcasting (5 wt. %), and spincasting (5, 10, and 20 wt. %) were evaluated. FIG. 38B shows the interferent screening properties of thick Nafion layers (5 wt. % drop cast) in a electrochemical cell comprising FePc 3DFG (working electrode), Pt (counter electrode), and Ag/AgCl (reference electrode) in N₂-saturated PBS. FIG. 38C shows the NO sensing calibration 3DFG sensor with a thin Nafion layer (5 wt. % spin cast). FIG. 38D is a summary plot of the sensitivity of the sensors to NO and interferent, NO₂, before and after Nafion casting with data given as mean±std, n=3.

### Example 22: Electrochemical Modification and Characterization of Planar Platinum Electrodes

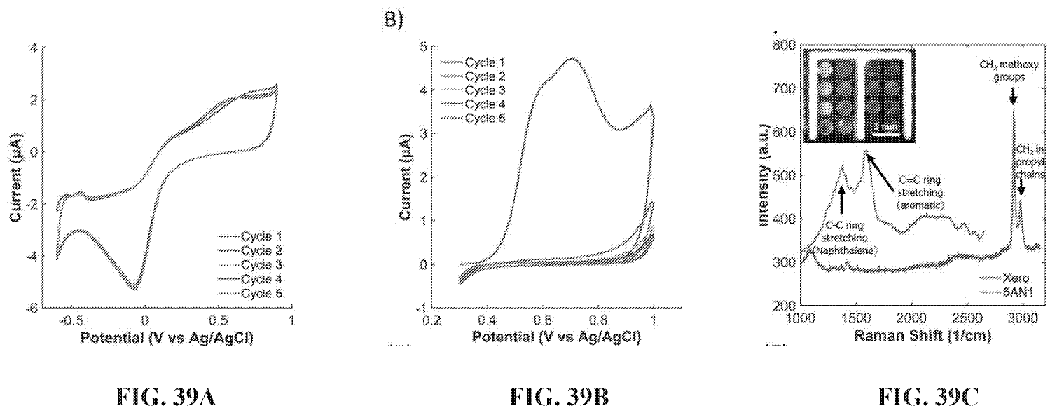

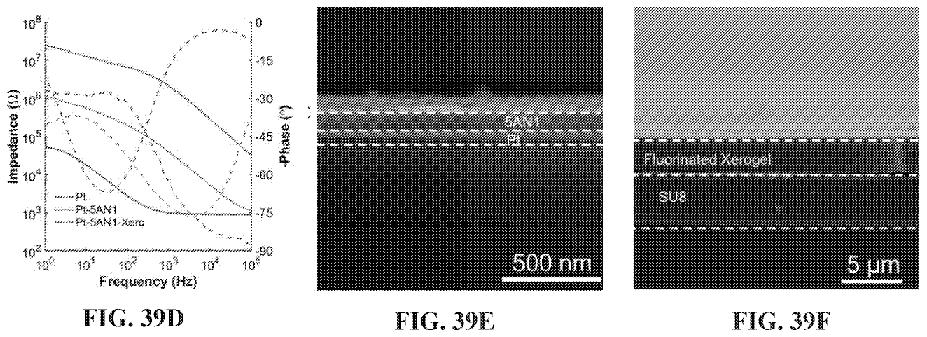

In this example, planar platinum electrodes are modified with a combination of 5-amino-1-napthol (5AN1) and a fluorinated Xerogel and characterized. FIG. 39A shows representative cyclic voltammograms of a platinum electrode for 5 cycles acquired with 1×PBS at a scan rate of 100 mV/s. The successful electropolymerization of 5AN1 was performed by cyclic voltammetry as shown in FIG. 39B. Subsequent coating of xerogel was then determined by Raman spectroscopy as shown in FIG. 39C. Impedance measurements were performed on each of bare Pt, Pt-5AN1, and Pt-5AN1-xerogel electrodes (FIG. 39D). FIGS. 39E-F are representative electron microscopy images showing the thickness of different electrode modifications of the example.

### Example 23: Synthesis of Electrodes Modified with Platinum Black and Characterization Thereof

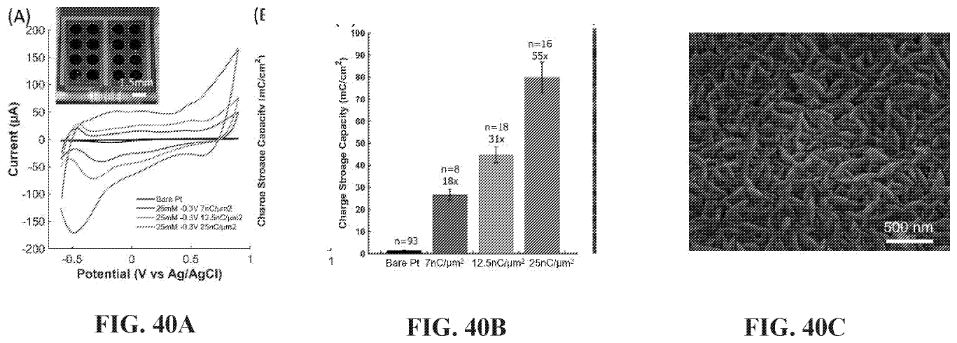

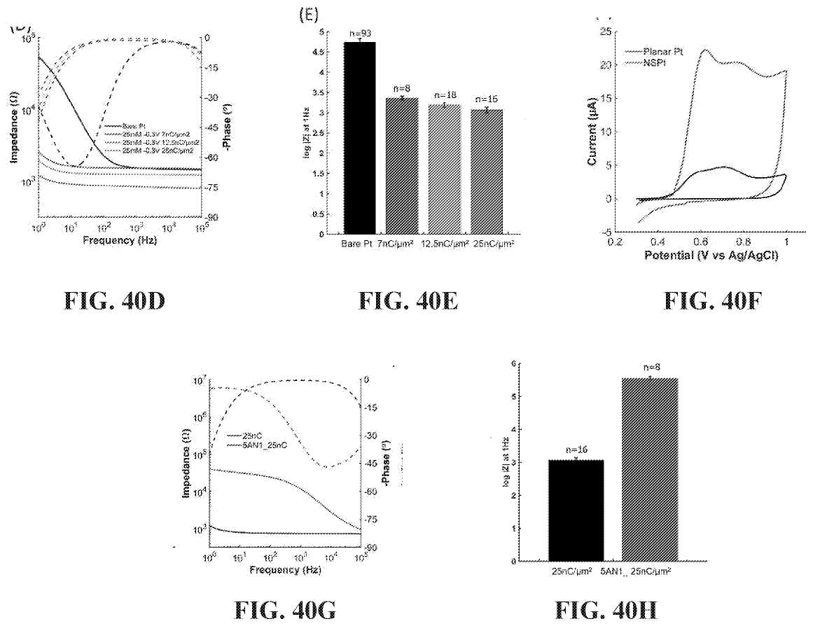

In this example, platinum black, a nanostructured platinum, was deposited and used as an electrode. Electrochemical and surface characterization such as cyclic voltammetry (FIG. 40A), charge storage capacity (FIG. 40B), scanning electron microscopy (FIG. 40C), and impedance spectroscopy (FIG. 40D-E) were performed at different deposition charge densities. 5AN1 was deposited on both planar and nanostructured platinum electrodes as verified by cyclic voltammetry (FIG. 40F). The effect of the selective 5AN1 layer on electrochemical characteristics was evaluated (FIGS. 40G-H).

### Example 24: Semipermeable Electrodeposited Coatings for Selective Electrochemical Sensing of NO

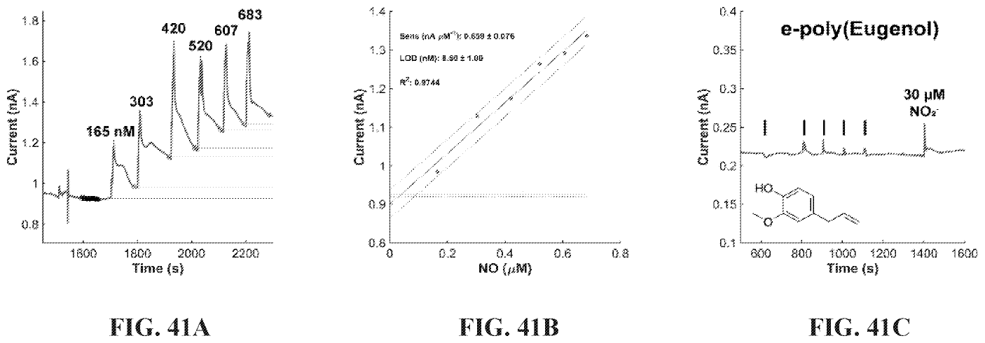

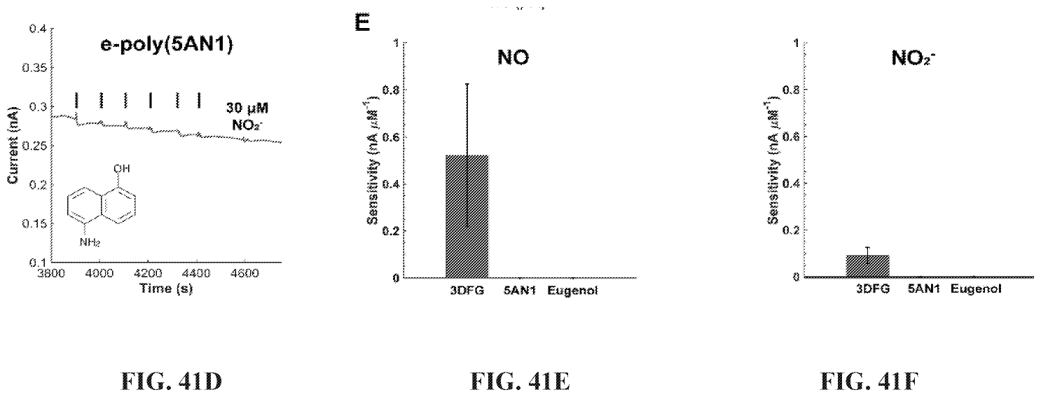

In this example, various electropolymerized polymers can be used as selective layer for nitric oxide sensing, including Eugenol, 5AN1 as shown in FIGS. 41A-D. Sensitivity of NO sensing were compared by using different electropolymerized polymer as selective layer as shown in FIGS. 41E-F.

### Example 25: In Vitro NO Sensing Calibration in the Controlled Setup

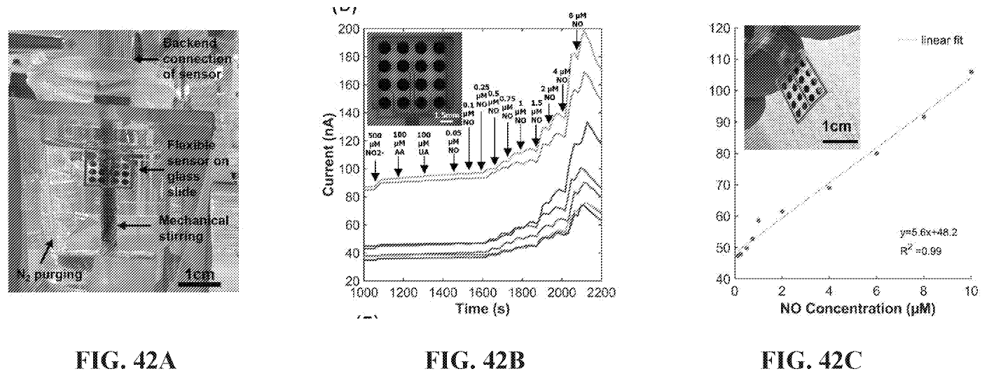

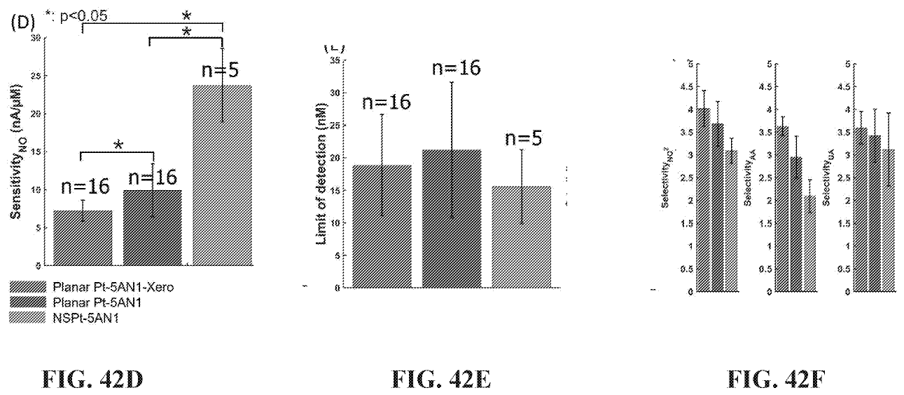

In this example, planar platinum and platinum black (nanostructured platinum) electrodes are modified with various combinations of coatings including 5AN1 and Xerogel. Representative calibration setup is shown in FIG. 42A; Representative 8-channel multiplexed chronoamperometry calibration in 1×PBS and in response to NO solutions and interferent solutions of nitrite, ascorbic acid and uric acid; is shown in FIG. 42B. A representative linear regression plot for NO detection is shown in FIG. 42C. As shown in FIGS. 42D-F, nanostructured platinum coated with 5AN1 shows a 3-fold increase in sensitivity and a lower limit of detection (LOD) towards nitric oxide compared to planar platinum.

### Example 26: In Vitro NO Sensing Experiments with RAW Macrophages

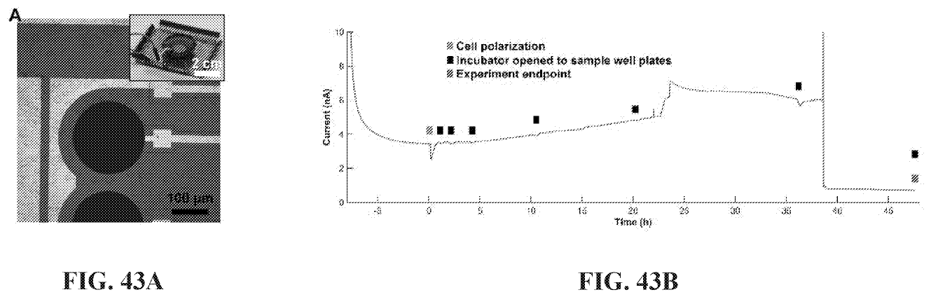

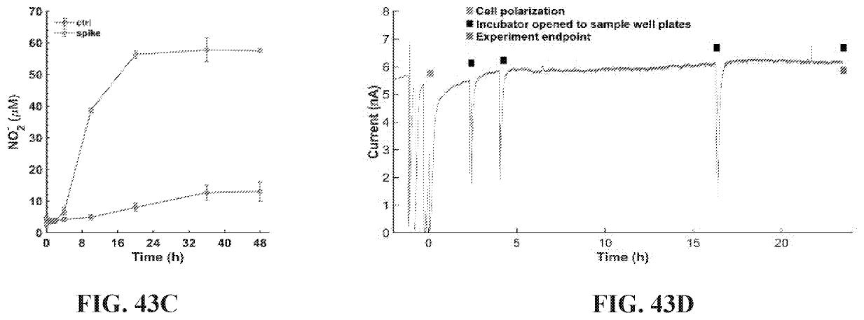

In this example, the efficacy of 3DGF working electrodes to detect NO release from a macrophage cell line in vitro is demonstrated. A microelectrode array capable of holding 2 mL of cell culture medium and 10⁶ RAW macrophage cells was designed and cell adhesion was demonstrated by optical microscopy (FIG. 43A and inset) Continuous current readings were performed on the system following polarization with IFN-γ and LPS (FIG. 43B) and compared to the continuous current measurements from a World precision instruments (WPI) nitric oxide probe (FIG. 43D). The Griess test was performed to measure the nitrite concentration from the polarized macrophages (FIG. 43C)

### Example 27: Multiplexed Electrochemical NO Detection from 8 Sensors

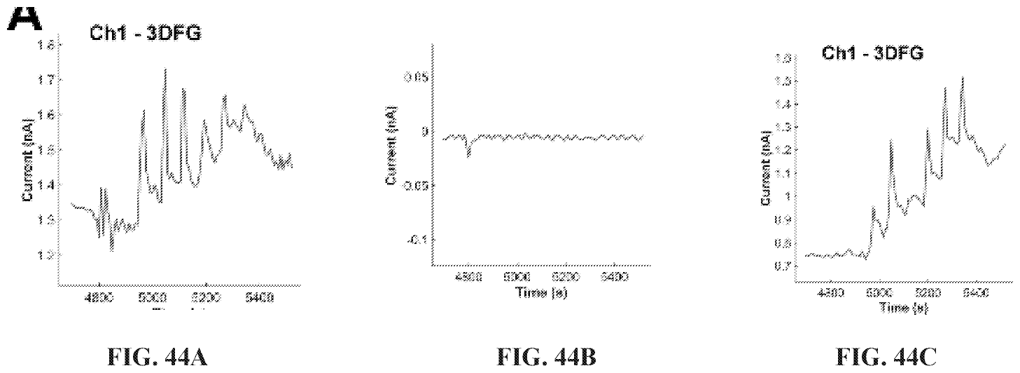

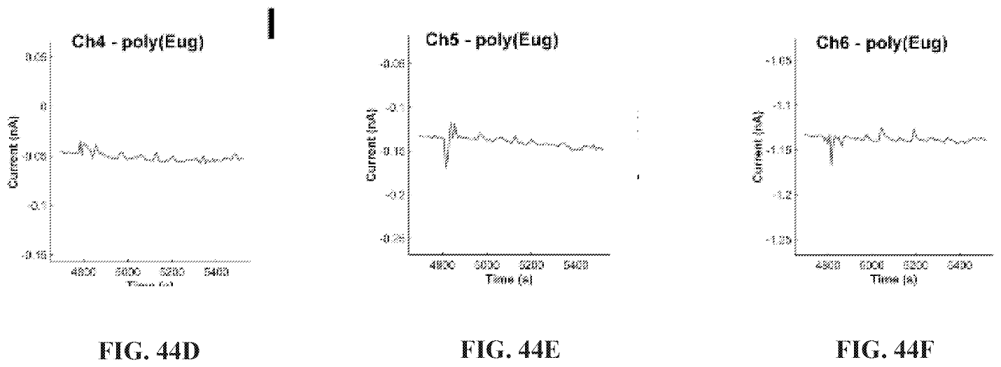

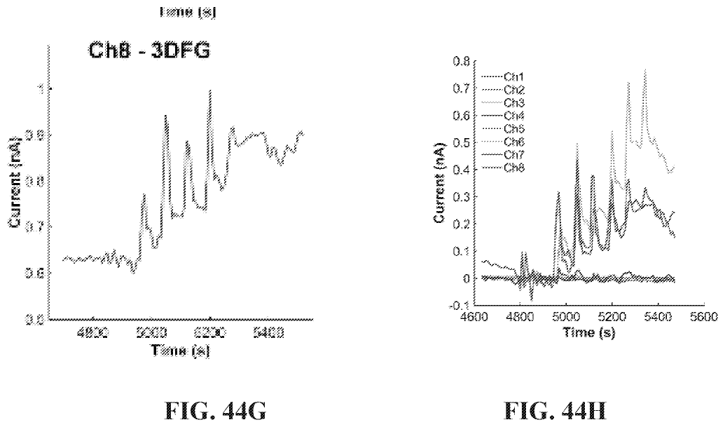

In this example, the ability to multiplex electrodes with different surface characteristics on the same array is demonstrated. An 8-electrode array with surfaces selected from FePc 3DFG (Channel 1, 3, and 8) or poly(Eug) 3DFG (Channel 4, 5, and 6) WEs, Pt CE, and Ag/AgCl RE was tested in N₂-saturated PBS. The electrode responses are shown individually in FIG. 44A and overlayed in FIG. 44B.

### Example 28: Poly(5AN1) Coatings on Pt Macroelectrode Arrays

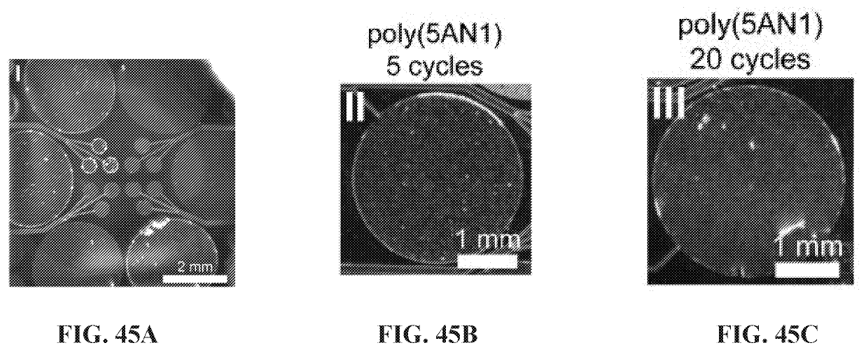

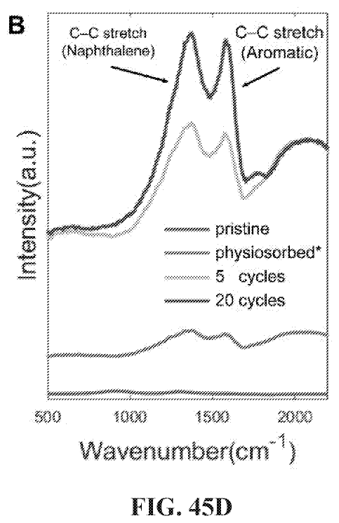

In this example, the ability to coat platinum electrodes with 5AN1 at different numbers of electropolymerization cycles is demonstrated. Optical images of a 3-mm Pt electrode before and after different numbers of electropolymerization cycles are shown in FIGS. 45A-C. Raman spectroscopy was performed to confirm the presence of 5AN1 (FIG. 45D).

### Example 29: Sensitive and Selective NO Detection after Coating Pt with Poly(5AN1)

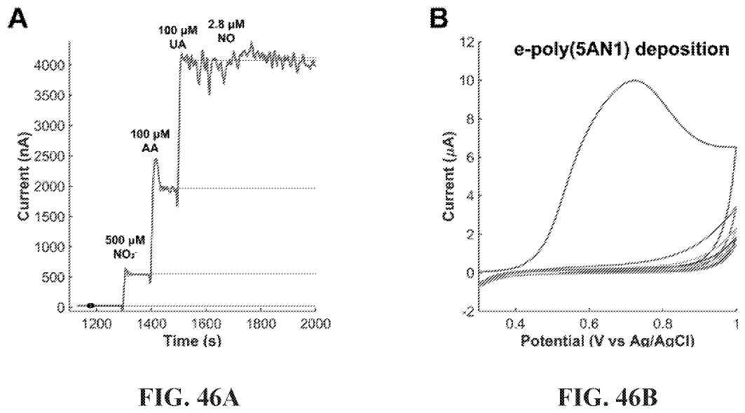

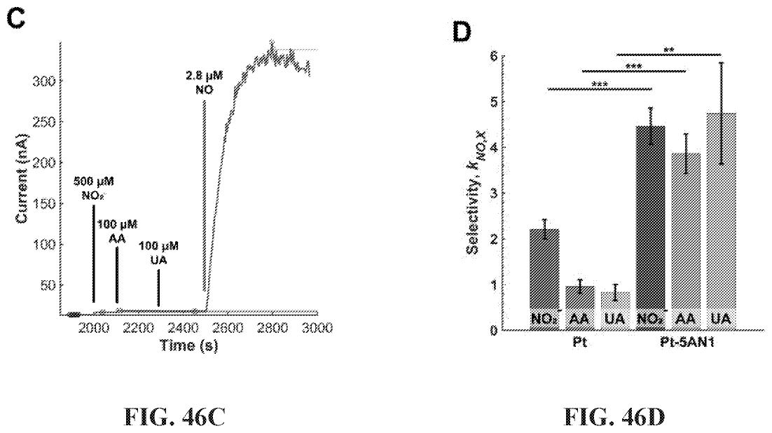

In this example, electrochemical methods are used to polymerize 5AN1 and measure nitric oxide concentration. NO selectivity against interferents nitrite (NO₂⁻), ascorbic acid (AA) and uric acid (UA) was measured before FIG. 46A and after FIG. 46C electrodeposition of selective polymer layers, poly(5AN1). There is a strong current response for interferants on the bare electrode, which is greatly reduced following coating with 5AN1. The deposition of 5AN1 on Pt 3-mm electrodes was verified by cyclic voltammetry performed from 0.3 to 1 V vs Ag/AgCl at 10 mV s⁻¹ for 5 cycles (FIG. 46B). The selectivity of the two electrodes for nitric oxide versus different known interferents is summarized in FIG. 46D.

### Example 30: Low NO Detection Limit of Poly(5AN1) 3DFG Electrodes

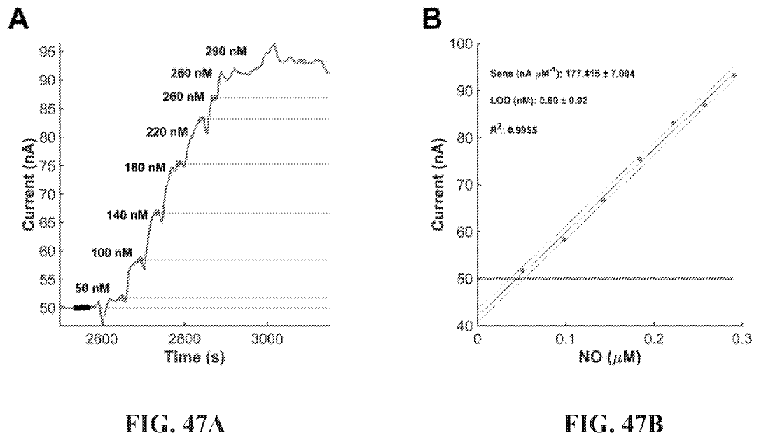

In this example, the ability of poly(5AN1) 3DFG electrodes to detect nitric oxide at low concentrations is demonstrated. Current readings were recorded during chronoamperometry measurement as shown in FIG. 47A. The peak current was plotted against cumulative nitric oxide concentration and a linear response was found (FIG. 47B). The limit of detection was determined to be 0.60+/−0.02 nM.

### Example 31: Standardization and Comparison with Commercial NO Probe

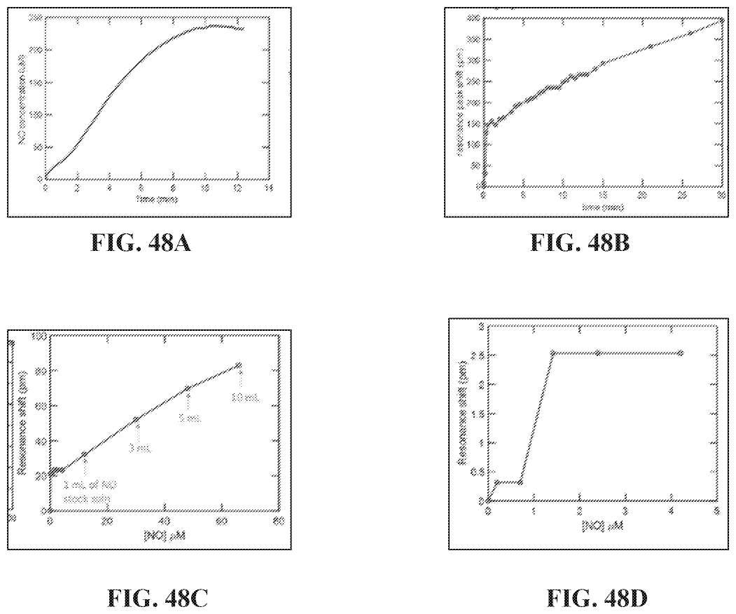

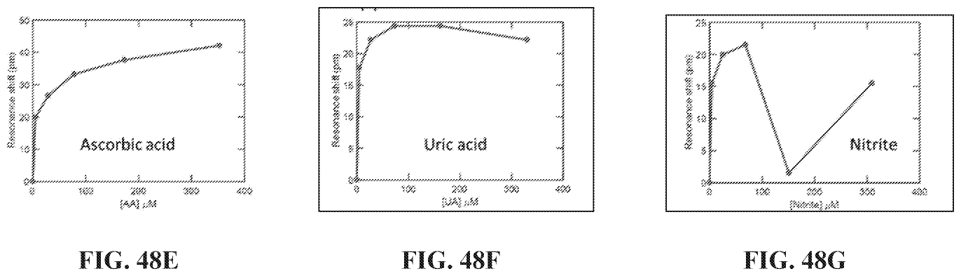

In this example, a commercial probe was used to measure the nitric oxide release profile from different sources. The response of the commercial probe to NO released from diethylamine NONOate (DEA NONOate; a known nitric oxide donor) is shown in FIGS. 48A-B. Additionally, low concentration NO detection by the commercial probe device was shown in a NO bubbling system (FIGS. 48C-D). Finally, the current response from interferents such as ascorbic acid, uric acid and nitrite were measured by the commercial device as shown in FIGS. 48D-F.

Keefer, Larry K., et al. ““NONOates” (1-substituted diazen-1-ium-1,2-diolates) as nitric oxide donors: convenient nitric oxide dosage forms.” Methods in enzymology. Vol. 268. Academic Press, 1996. 281-293.

### Example 32: Flexible Sensor Array Fabrication and Characterization

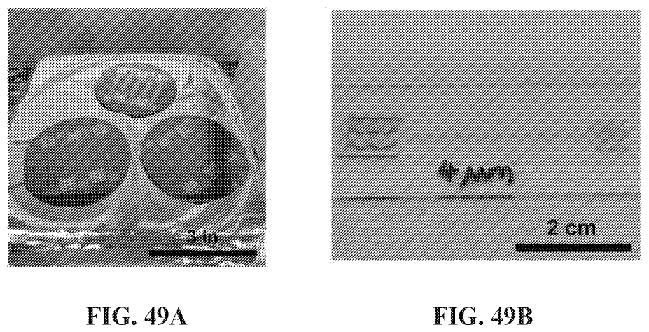

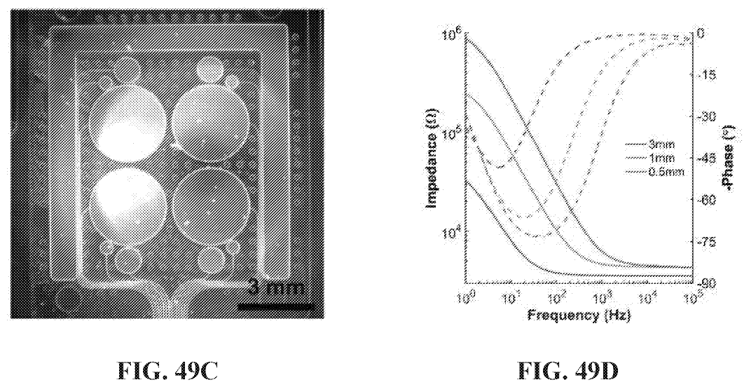

In this example, flexible electrode arrays were fabricated by standard clean room nanofabrication techniques such as spin coating of photoresist, photolithography and metal deposition. As shown in FIG. 49A, wafer-scaled fabrication of flexible sensor arrays on Si/600 nm SiO₂ substrate were synthesized. A flexible sensors comprising different sized electrodes was imaged on a glass slide (FIG. 49B) and a zoomed-in image of the electrode area was taken (FIG. 49C). Electrochemical impedance spectroscopy was performed to compare the impedance and phage angle of various size of electrodes as shown in FIG. 49D.

### Example 33: Flexible NO Sensor Array Ex Vivo Demonstration

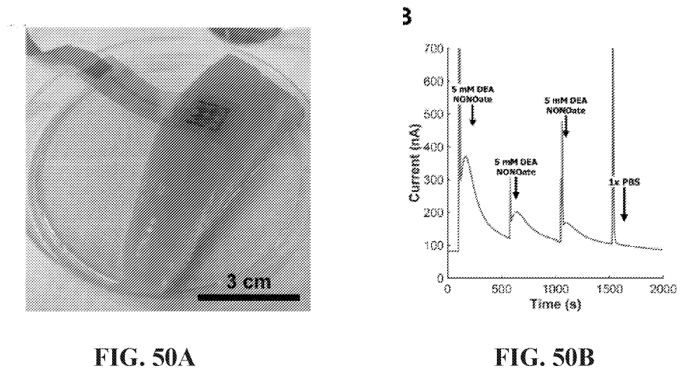

In this example, the ability of the flexible electrode arrays disclosed herein to detect nitric oxide in an ex vivo setting was demonstrated. As shown in FIG. 50A, the flexible electrode arrays were successfully affixed to a chicken breast sample. DEA NONOate was added to the ex vivo sample and continuous current readings were taken (FIG. 50B).

### Example 34: Flexible NO Sensor Array Ex Vivo Rodent Wound Model

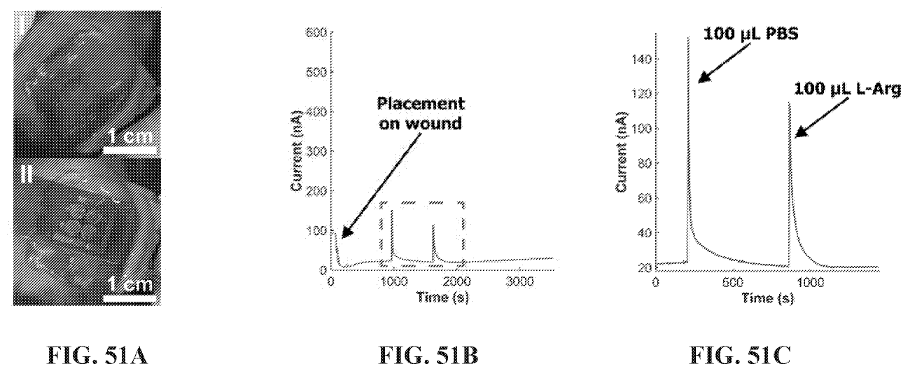

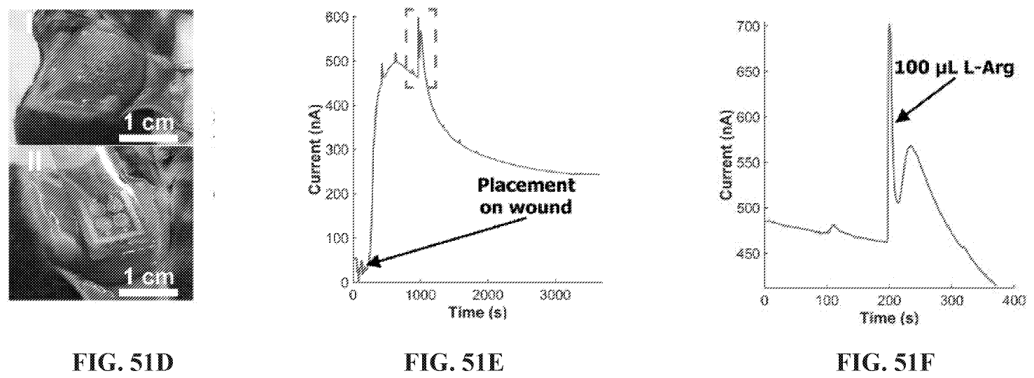

In this example, the ability of the flexible electrode arrays disclosed herein to detect nitric oxide in an ex vivo rodent wound model was demonstrated. As shown in FIG. 51A, the flexible electrode sensor was successfully affixed to a rodent wound model. FIGS. 51B-C shows the current response of the sensor to 100 μL aliquots of 1×PBS and 10 mM L-Arg. FIG. 51D shows an image of wound surface on ex vivo rodent with inflammatory polypropylene mesh implant without (I) and with (II) placement of the flexible NO sensor array. FIG. 51E-F shows the current response of the sensor from the inflammatory mesh for 1 h.

### Example 35: Demonstration of In Vivo Nitric Oxide Sensing in a Canine Subject

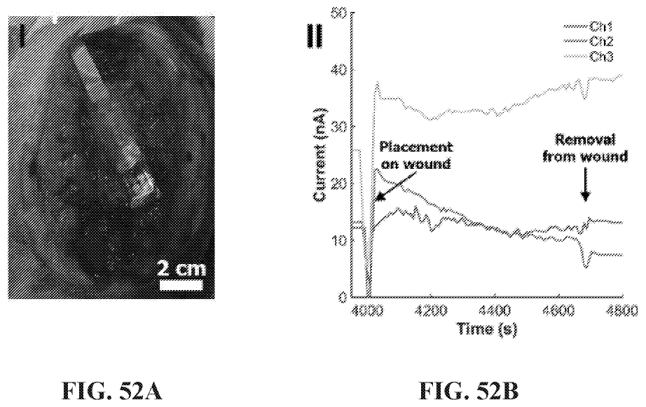

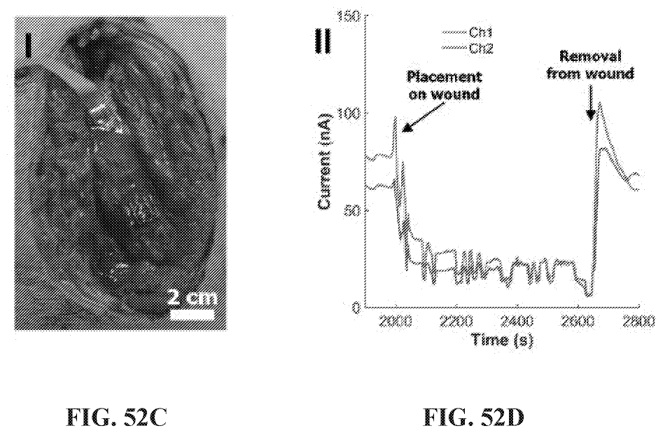

In this example, the ability of the flexible electrode arrays disclosed herein to detect nitric oxide in an in vivo canine model was assessed. The flexible electrode array of Example 32 was added to a model of a volumetric muscle loss wound as shown in FIG. 52A. A multiplexed three sensor reading was taken for ten minutes following placement on the wound as shown in FIG. 52B to detect nitric oxide. Twenty-four hours after the VML injury, the flexible electrode array was added back to the wound (FIG. 52C) and a multiplexed two-sensor was taken for 10 minutes (FIG. 52D) to detect nitric oxide.

### Example 36: Demonstration of In Vivo Nitric Oxide Sensing in a Canine Subject Over Extended Time Frames

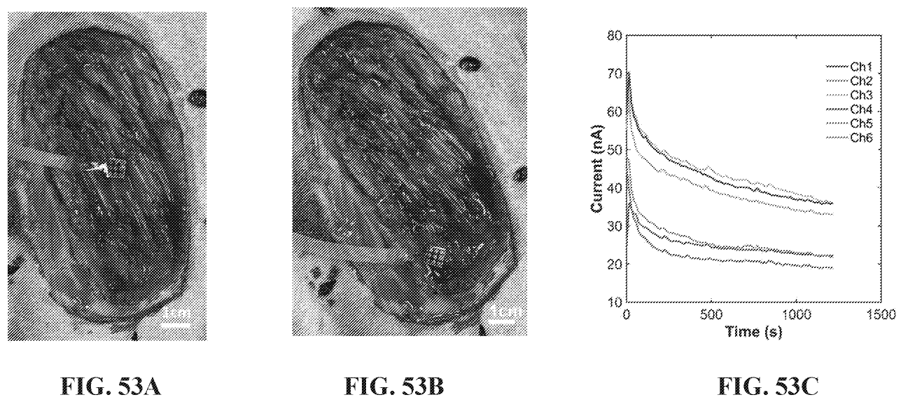

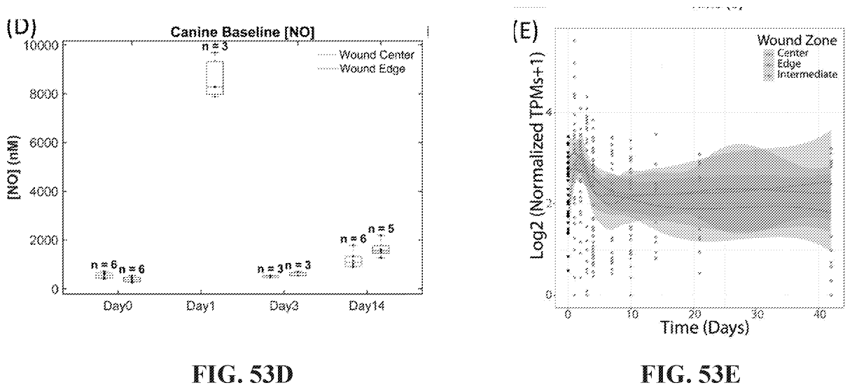

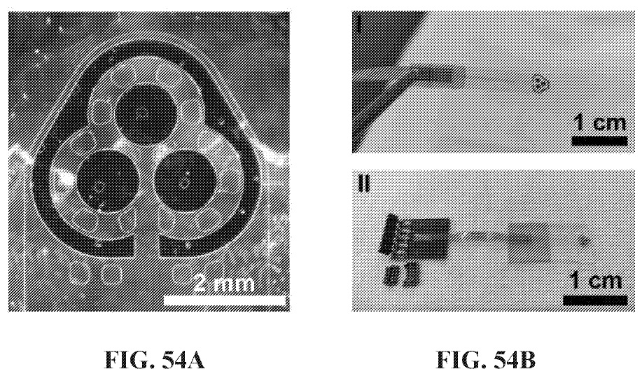

In this example, a flexible, 6-channel electrode array was added to a volumetric muscle loss wound in a canine subject and NO concentrations were measured. FIGS. 53A-B show photographs of the flexible arrays on the center and edge of the canine wound, respectively. Representative current responses of the electrodes are shown in FIG. 53C, and the concentration of NO over the course of 14 days from both center and edge sites was collected (FIG. 53D). In addition, gene expression analysis was performed on biopsy samples at locations where NO sensors were placed (FIG. 53E).

### Example 37: Demonstration of Small Form Factor NO Sensor for In Vivo Applications

In this example, a small form factor nitric oxide sensor was designed for use in mouse wound model. An optical image of the small form factor model is shown in FIG. 54A comprising an array of three 1 mm platinum electrodes. Additional images of the small form factor model are shown in FIGS. 54B-C, wherein an ACF cable bonds to a small custom printed circuit board with pin headers (or Molex Pico Ezmate) connectors to shielded electrical cabling. This example demonstrates the feasibility of miniaturization and customization of the flexible electrodes arrays described in previous examples.

### Example 38: Polymer Coating of Small Form Factor NO Sensors

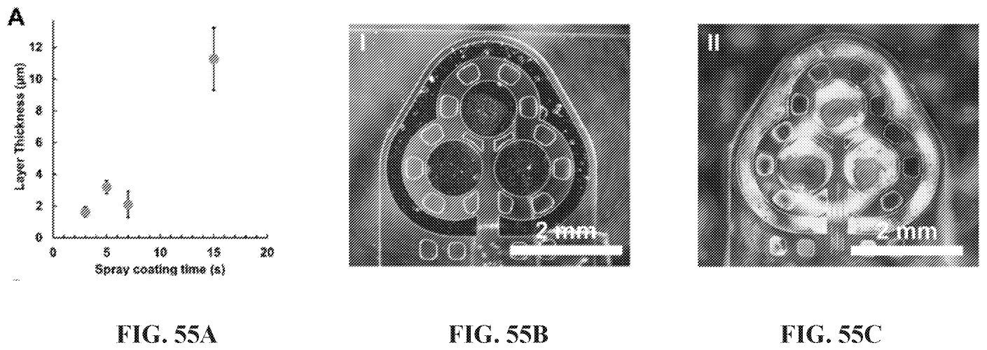

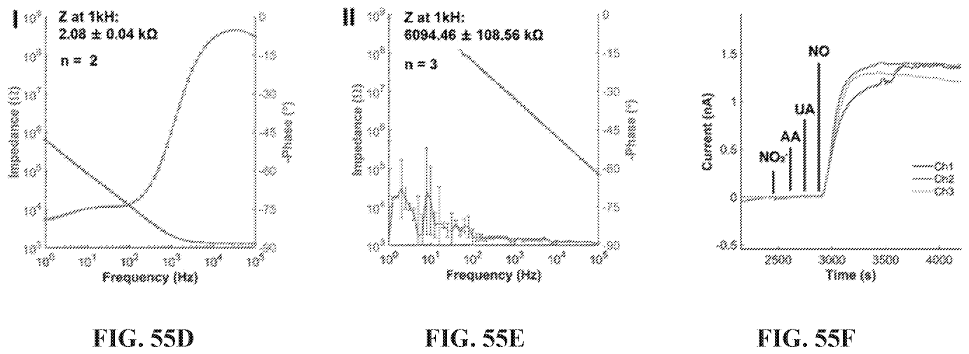

In this example, the small form factor electrode arrays of Example 37 were coated with organic polymers and evaluated for selectivity and sensitivity for NO detection. Electrodes coated with 5AN1 were coated with xerogel and evaluated for layer thickness as a function of spray coating time (FIG. 55A). Optical images of the electrodes before and after coating with xerogel are shown in FIGS. 55B-C. Additionally, the small form factor nitric oxide sensor was characterized by electrochemical impedance spectroscopy before and after coating with xerogel (FIGS. 55D-E). The nitric oxide sensor was also calibrated by adding interferents and NO (FIG. 55F).

### Example 39: Evaluation of the Stability of NO Sensors Under Physiological Conditions

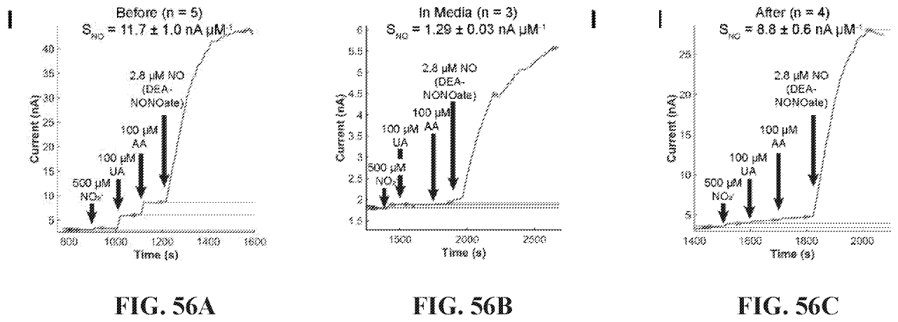

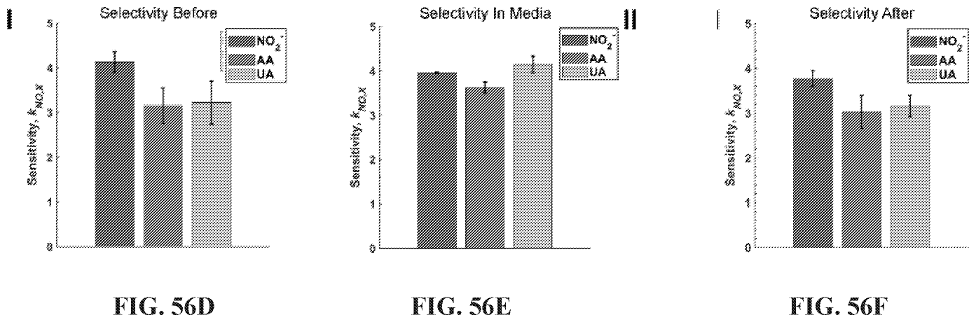

In this example, the stability of the NO sensors described herein were compared in different incubation conditions. Calibration curves of 1 mm Pt-5AN1 sensors with 3 aliquots of interferent (NO₂⁻, uric acid (UA), ascorbic acid (AA)) and 1 aliquot of NO (DEA-NONOate) were collected and selectivity was evaluated. Three conditions were used: 1×PBS (S_{NO}=11.7±1.0 nA μM⁻¹. n=5) (FIG. 56A-B); serum-containing media (S_{NO}=1.29±0.03 nA μM⁻¹, n=β) (FIGS. 56C-D); and complete media for 24 hours at room temperature followed by DI water wash and evaluated in 1×PBS (S_{NO}=8.8±0.6 nA μM⁻¹, n=4) (FIGS. 56E-F).

### Example 40: Fabrication of a Patterned Pseudo-Reference Ink-Based Electrode

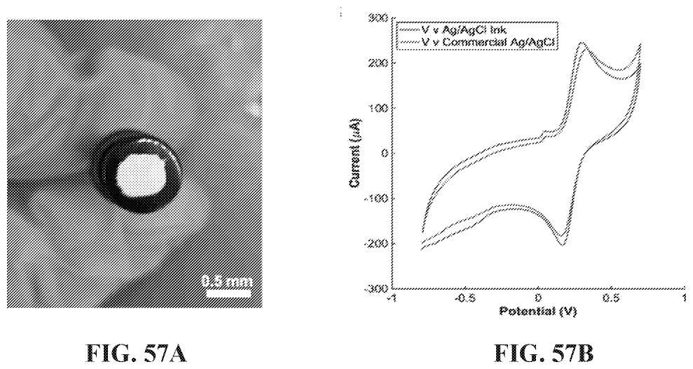

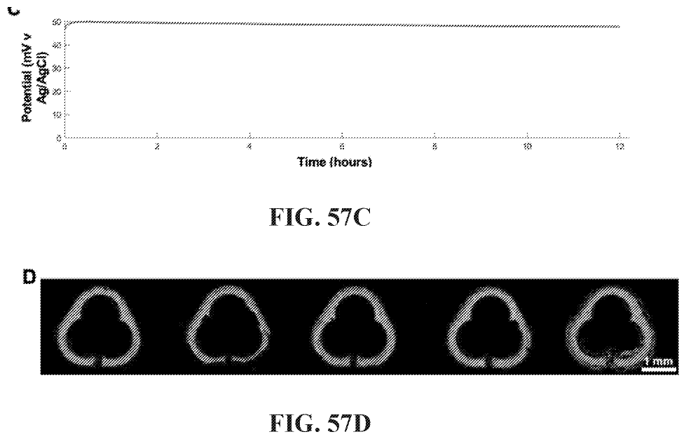

In this example, an on-chip reference electrode was fabricated by screen printing of sliver silver-chloride ink. The Au electrode coated with Ag/AgCl ink for electrochemical characterizations is shown in FIG. 57A. The screen-printed electrode was compared to a commercial Ag/AgCl reference electrode by cyclic voltammetry of glassy carbon in 1 mM [Fe(CN)₆]³⁻ as shown in FIG. 57B. Further, the open circuit potential between commercial Ag/AgCl reference electrode and the pseudo-reference/counter electrode with a potential drift over 12 hours of 0.001 mV/min is shown in FIG. 57C. Stereoscope image of microbrush (Nicpro Flat 2) painted pseudo-reference-/counter-electrode onto a glass slide using a 1 mil PET film stencil, cut using LPKF Protolaser is shown in FIG. 57D. The glass slide was heated to 80° C., Ag/AgCl ink was painted over each individual cut-out from bottom to top of design, cured for 10 min at 110° C., and the stencil was removed post-cure. The average thickness of the Ag/AgCl coating was 59.9±12.0 μm.

### Example 41: Selectivity and Sensitivity of 5AN1-Au-Based Sensors

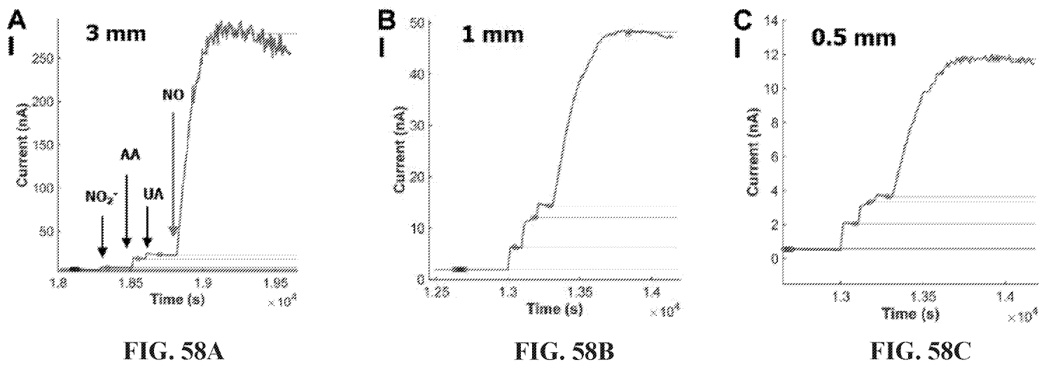

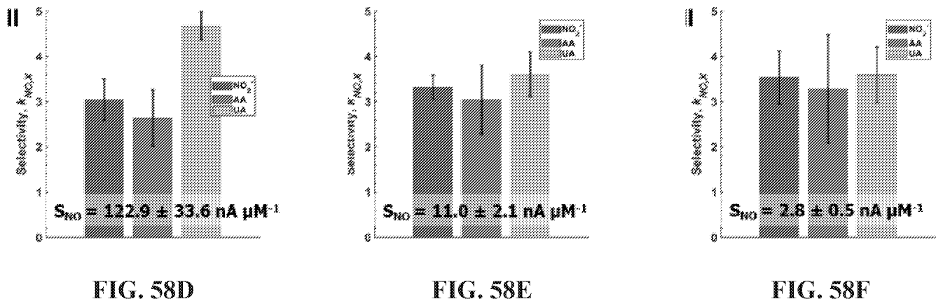

The example describes the characterization of 5-AN1-Au-NO sensors as a function of electrode size. Briefly, the selectivity and sensitivity of 5-AN1-Au-based nitric oxide sensors was determined as a function of electrode diameter in the presence of interferents and NO. The sensors of diameter 3 mm, 1 mm and 0.5 mm were first calibrated in the presence of 1×-strength phosphate-buffered saline (PBS) containing three aliquots of each of the following interferents: NO2−, ascorbic acid (AA), uric acid (UA), and one aliquot of NO (DEA NONOate), as shown in FIGS. 58A, 58B, and 58C for 3 mm-, 1 mm-, and 0.5 mm-diameters, respectively. Current amplitude (nA) is plotted over time after addition of interferents and NO. Currents elicited by interferents are negligible in magnitude compared to currents elicited by NO; however, they become more significant at smaller electrode diameters. The selectivity of the 5-AN1-Au-based-NO electrodes as a function of size was subsequently determined, as illustrated in FIGS. 58D-F. The 3-mm diameter electrode was measured to have a selectivity for nitric oxide over interferents of 122.9±33.6 nanoamperes per micromole per liter (nA·μM-1), whereas the 0.5-mm diameter electrode had a measured selectivity for nitric oxide over interferents of 2.8±0.5 nA·μM⁻¹. All results are presented as mean±standard deviation. The 3 mm diameter 5-AN1-Au-based nitric oxide sensor, therefore, had the highest calculated selectivity.

### Example 42: In Vivo Rodent Acute NO Sensing

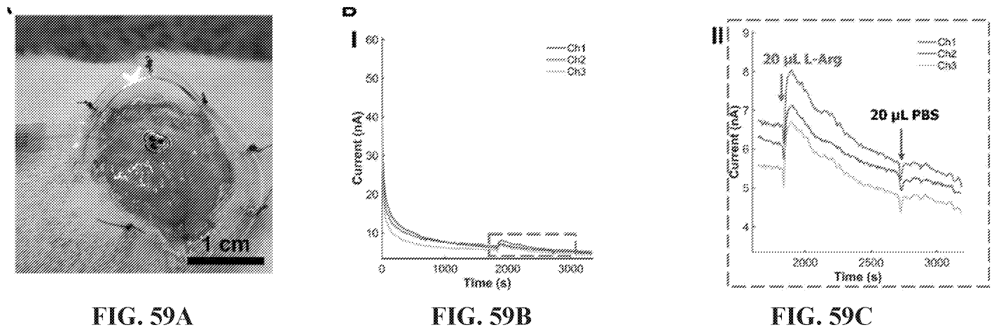

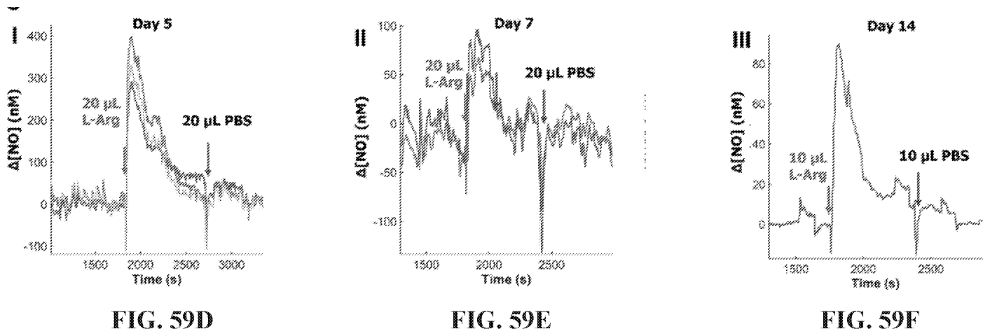

The example set forth below delineates the acute sensing of NO in an in vivo rodent wound model by induction with L-arginine, which stimulates endogenous NO production, to confirm NO response. Five days after wound creation, an embodiment of the invention comprising an NO sensor array was placed on the rodent wound, as depicted in FIG. 59A. NO production was induced with L-Arginine in 1× phosphate-buffered saline (PBS) at Day 5 (cf. FIG. 59D), Day 7 (cf. FIG. 59E), and Day 14 (cf. FIG. 59F). Three multiplexed 1-mm diameter sensors recorded current induced from the VML injury model for about 1 hour at Days 5, as shown in FIGS. 59B-C. Baseline subtraction of recorded current was performed with 8-points exponential function fitting and the NO concentrations were converted with NO sensitivity of each sensor.

### Example 43: Rat Wound Model In Vivo NO Sensing

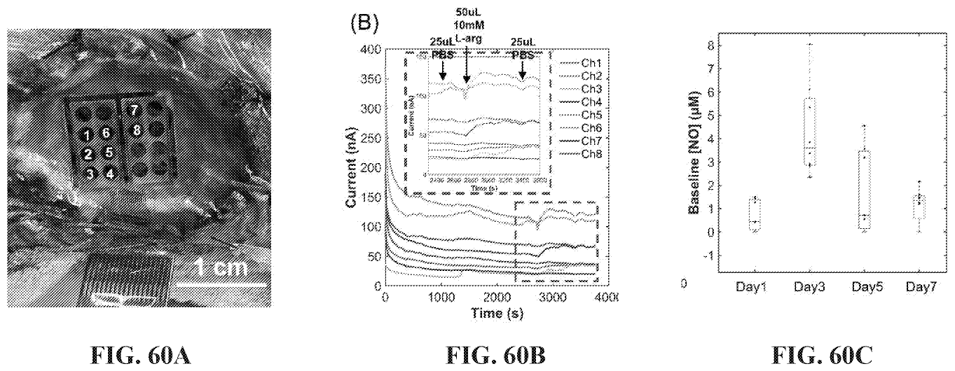

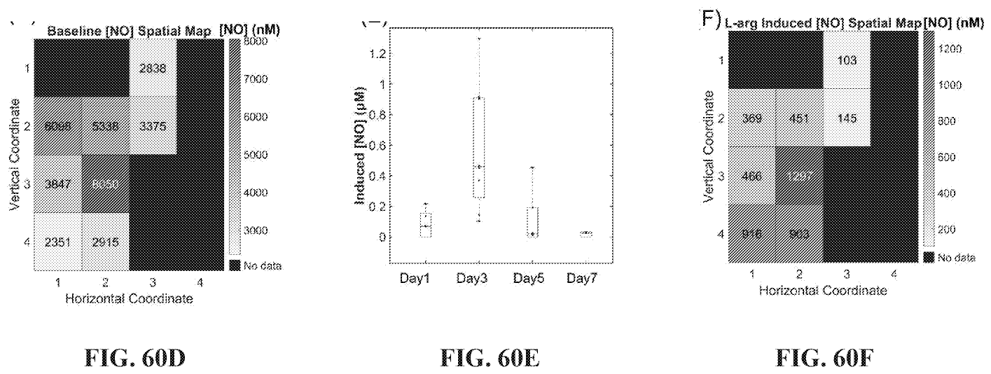

An alternative example is provided herein for in vivo acute measurement of nitric oxide from a rat wound model. Wounds were created on Day 0 and nitric oxide sensing was measured on Day 1, 3, 5, and 7 post-surgery. An embodiment of the invention comprising a flexible, multiplexed NO sensor array with eight (8) channels was placed on the rat wound, as depicted in FIG. 60A, which allows for chronoamperometric (i.e., current response over time) and spatiotemporal maps of NO concentration, [NO], to be generated. NO response was measured at Day 1, Day 3, Day 5, and Day 7 with NO induction by L-Arg in IX PBS on Day 3. Chronoamperometric measurements of baseline [NO] and L-Arg-induced [NO] are given in FIGS. 60C and 60E. Due to the design of the 4×4 multiplexed array, the chronoamperometric data gathered from each electrode can be concatenated to create spatiotemporal maps of [NO] over the area of the wound bed. FIGS. 60D and 60F show the baseline [NO] and L-Arg-induced [NO] spatiotemporal maps, respectively. By using the nitric oxide sensor, both baseline and induced nitric oxide peaked on Day 3. Spatial mapping of nitric oxide suggests a higher nitric oxide concentration near the center of the wound center rather than the near the wound periphery.

### Example 44: Δ[NO] after L-Arginine Induction as Function of Time Post-Surgery

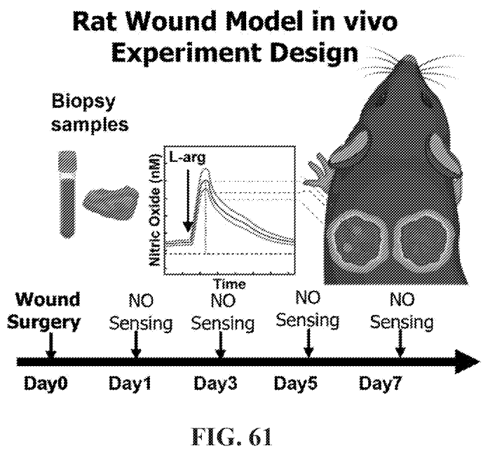

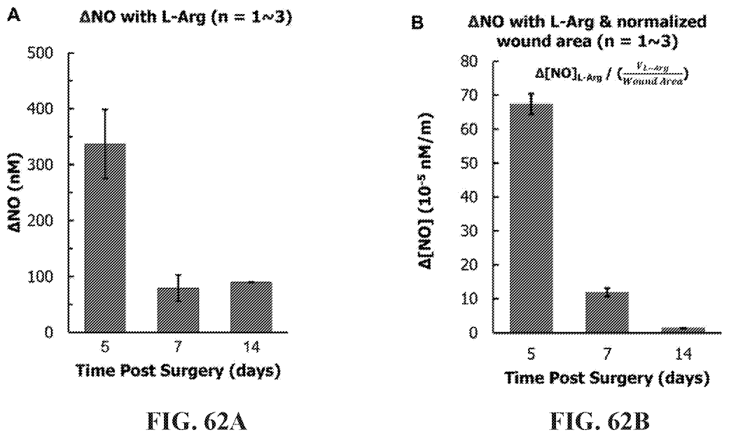

This example describes the measurement of change in the nitric oxide concentration, Δ[NO], after L-Arginine induction as a function of time post-surgery in a rodent wound model (FIG. 61). The wound was created on Day 0 and the NO sensor array was placed in the wound bed on Day 1. L-Arginine in 1×PBS was then introduced into the wound bed to stimulate NO production on Day 3. Stimulation of NO production was then measured chronoamperometrically on Day 5, Day 7 and 14. FIG. 62A is a plot of Δ[NO] at the aforementioned time points following surgery Further, normalized measurements were considered employing the size of the wound area. FIG. 62B is a plot of the change in the L-Arginine-induced nitric oxide production normalized by L-Arginine volume per wound area. L-Arginine-induced NO production peaked rapidly and then gradually attenuated days post-surgery. Data for both graphs are presented as average±standard deviation (n=1-3, no standard deviation is presented if n=1).

### Example 45: NO Sensor Array Performance Fidelity Following In Vivo Recording

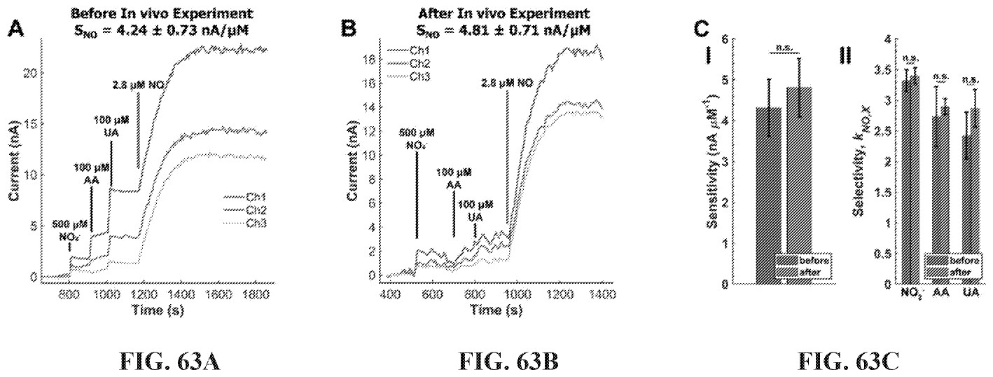

The performance fidelity of an embodiment of the NO sensor array was investigated as delineated herein: the NO sensor performance was measured prior to and following an in vivo recording of a rodent wound model. Chronoamperometric plots of current magnitudes elicited after exposure to NO and interferents exposure on a three-channel array, as shown in FIGS. 63A-B, before and after the in vivo wound model experiment suggest that the NO sensor array maintained a similar level of NO detection. FIGS. 63CI-II are plots of the NO sensitivity in nA·μM⁻¹ and selectivity kNO,X against interferents NO₂⁻, ascorbic acid (AA), and uric acid (UA), both before and after the in vivo wound model experiment. One-way ANOVA and post-hoc Tukey indicate that there was no significant difference in the NO sensitivity and NO selectivity against interferents measures both before and after the in vivo experiment. The NO sensor array performance was therefore determined to be robust.

### Example 46: High Density Sensor Array Design for Multiplexing and Dual-Sensing

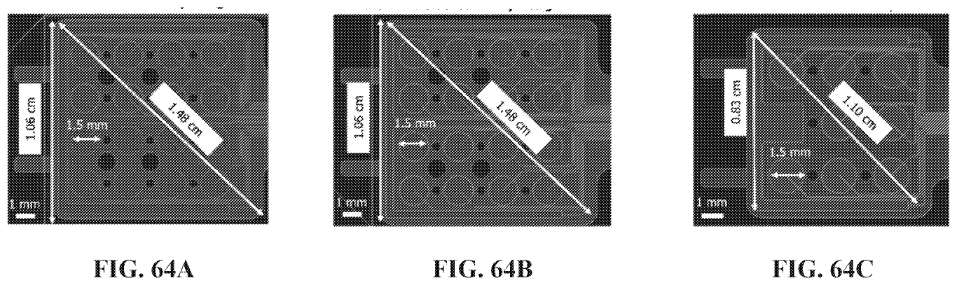

Described in the example set forth below is the design of high-density sensor array embodiments for multiplexing and dual sensing of analytes present in the wound bed. FIGS. 64A-B give schematics of two alternative 16-channel arrays with two different reference and counter electrode configurations. Both configurations are 1.06 cm on a side and 1.48 cm diagonally. FIG. 64C illustrates a third, 8-channel sensor array measuring 0.83 cm on a side and 1.10 cm diagonally. These designs have been implemented in animal wound models which would yield chronoamperometric plots and spatiotemporal maps of analyte production, e.g., NO production. It is conceived that the present designs have the capability to measure more than one analyte simultaneously. For instance, a high-density sensor array may have the capability to measure nitric oxide, a chemokine or a cytokine, e.g., TGF-β, independently or in combination simultaneously.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. A wound healing device comprising:
(i) a first component comprising a well/depression comprising a polymer (e.g., a biocompatible polymer);
(ii) a second component; and
(iii) a cell.

2. The wound healing device of claim 1, wherein said first component comprises a needle array.

3. The wound healing device of claim 2, wherein a pillar array is formed from first and second components, said first component comprising a chamber for housing the cell or cells, and a second component providing structural support to said first component (e.g., a base).

4. The wound healing device of claim 1, further comprising one or more electrode arrays, wherein the one or more electrode arrays is capable of providing electrical stimulation or detect a biomarker, wherein the biomarker is nitric oxide, a chemokine or a cytokine, e.g., TGF-β.

5-6. (canceled)

7. The wound healing device of claim 1, wherein the cell is an engineered cell (e.g., a living engineered cell), wherein the engineered cell comprises an exogenous transcription unit for expression of a substance, e.g., a polypeptide or nucleic acid.

8. (canceled)

9. The wound healing device of claim 7, wherein the exogenous transcription unit comprises the coding sequence of a polypeptide, wherein the exogenous transcription unit further comprises a promoter operably linked to the coding sequence of the polypeptide.

10. (canceled)

11. The wound healing device of claim 9, wherein the polypeptide is activated upon exposure to a stimulus.

12. The wound healing device of 9, wherein the polypeptide is expressed constitutively.

13. The wound healing device of claim 11, wherein the stimulus is light or a small molecule, wherein the light is visible light (e.g., having a wavelength between 400-700 nm, e.g., blue light or red light).

14. (canceled)

15. The wound healing device of claim 9, wherein the polypeptide is a growth factor, hormone, cytokine, enzyme, antibody, clotting factor, or neurotransmitter, wherein the growth factor is brain derived neurotrophic factor (BDNF) or nerve growth factor (NGF).

16. (canceled)

17. The wound healing device of claim 15, wherein the cytokine is tumor necrosis factor-α (TNF-α) or interleukin-4 (IL4).

18. The wound healing device of any of claim 9, wherein the substance is produced in an amount greater than 0.01 pg/cell/hr.

19. (canceled)

20. The wound healing device of claim 1, wherein the first compartment comprises the cell or a plurality of cells.

21.-23. (canceled)

24. The wound healing device of claim 1, wherein the polymer of the first component comprises a naturally occurring polymer, e.g., a polysaccharide, e.g., alginate, hyaluronate, chrondroitin, chistosan, or dextran.

25. (canceled)

26. The wound healing device of claim 24, wherein the alginate is cross-linked, e.g., alginate-acrylamide or alginate-gelatin.

27. The wound healing device of claim 1, wherein the polymer of the first component is non-naturally occurring, e.g., a synthetic polymer, e.g., polyethylene glycol (PEG), polyurethane, PDMS, PTFE, polyethylene.

28. The wound healing device of claim 1, wherein the first component comprises between about 10 and 10,000,000 cells/ml of said first component.

29. The wound healing device of claim 1, wherein the cell is engineered to secrete a growth factor, a cytokine, a lymphokine, chemokine or neurotropic factor or hormone.

30. The wound healing device of claim 1, wherein the cell is engineered to secrete IL-4, BDNF, TNF-α, NGF, IL-12, IL-10, EGF, FGF-2, PDGF, VEGF, IL-1, IL-6, CTGF, GM-CSF, leptin, adiponectin, IP-10, NGF, IGF-1, dopamine, acetylcholine, fractalkine, HMGB1, IL-1β, IL-1RA, IL-2, sIL-2Rα, IL-5, IL-7, IL-8 (CCL8), IL-13, IL-15, IL-17A, IL-18, IFN-γ, IP-10 (CXCL10), MIG (CXCL9), MIP-1α (CCL3), MIP-1β (CCL4), MCP-1 (CCL2), M-CSF, Eotaxin (CCL11), active/latent TGF-β1, and/or lactic acid other metabolites for glycolysis.

31. The wound healing device of claim 1, wherein the cell is a mesenchymal stem cell, keratinocyte, fibroblast, chondrocyte, or retinal pigment epithelial cell.

32. The wound healing device of claim 1, further comprising an immunomodulatory agent in either or both the first and second component to mitigate immune responses against the device when placed into contact with living tissue in a subject.

33. The wound healing device of claim 1, further comprising biomarker sensor to map wound healing, wherein the biomarker sensor measures nitric oxide, a chemokine or a cytokine, e.g., TGF-β.

34. (canceled)

35. The wound healing device of claim 33, wherein said biomarker sensor provides a spatial map of healing in said wound, said sensor optionally being operably connected to a light emitting device.

36. The wound healing device of claim 1, further comprising an electrical stimulator, wherein said electrical stimulator is a low impedance/high charge injection stimulator.

37-42. (canceled)
